(19) United States
(12) Patent Application Publication
(10) Pub. No.: US 2024/0093244 A1
(43) Pub. Date: Mar. 21, 2024

(54) EUKARYOTIC CELLS FOR PROTEIN MANUFACTURING AND METHODS OF MAKING THEM

(71) Applicant: Selexis S.A., Plan-les-Ouates (CH)

(72) Inventors: Nicolas Mermod, Plan-Les-Ouates (CH); Pierre-Olivier Duroy, Plan-Les-Ouates (CH); Sandra Bosshard, Plan-Les-Ouates (CH); Philippe Le Mercier, Plan-Les-Ouates (CH)

(73) Assignee: Selexis S.A., Plan-les-Ouates (CH)

(21) Appl. No.: 18/351,819

(22) Filed: Jul. 13, 2023

Related U.S. Application Data
(62) Division of application No. 16/065,167, filed on Jun. 22, 2018, now Pat. No. 11,739,351.
(60) Provisional application No. 62/387,375, filed on Dec. 24, 2015.

(51) Int. Cl. C12N 15/90; C12N 5/071; C12N 15/10; C12N 15/113; C12N 15/861; C12P 21/00

(52) U.S. Cl. CPC C12N 15/907; C12N 5/0682; C12N 15/102; C12N 15/113; C12N 15/861; C12P 21/00; C12N 2310/20; C12N 2510/00; C12N 2511/00

(57) ABSTRACT

Disclosed are mammalian cells and mammalian cell lines that have a reduced load of remnants of past viral/retroviral infections and methods of producing and using the same.

### CROSS-REFERENCE TO RELATED APPLICATIONS

This is a divisional application of U.S. application Ser. No. 16/065,167, allowed, which is the U.S. national stage of international application no. PCT/EP2016/082567, filed Dec. 23, 2016 designating the United States and claiming the benefit of U.S. provisional application No. 62/387,375, filed Dec. 24, 2015, all of which are incorporated herein by reference in their entity.

### INCORPORATION OF AND REFERENCE TO ELECTRONIC SEQUENCE LISTING

The sequence listing was filed as a text file with international application no. PCT/EP2016/082567, filed Dec. 23, 2016 is hereby incorporated by reference in its entirety. The present application contains a Sequence Listing which has been submitted electronically in .XML format and is hereby incorporated by reference in its entirety. Said .XML copy, created on Jul. 10, 2023, is named “3024-232NSCON.xml” and is 122,491 bytes in size. The sequence listing contained in this .XML file is part of the specification and is hereby incorporated by reference herein in its entirety.

### BACKGROUND

Many stable, high yield mammalian cell lines used in recombinant protein manufacturing of therapeutic drugs are currently available. Processes have been optimized and improved so that cell line development could be reduced from 8 to 12 months to 4 months, while, at the same time, improving the yield and stability of protein-producing mammalian cell lines.

However, there remains a need to provide mammalian cell lines that have better safety profiles.

In particular, there is a general need to devise more efficient procedures for analyzing and documenting recombinant protein-producing mammalian cell lines, thus increasing throughput, speed and efficacy of candidate drug development, and decreasing the costs of regulatory validation as well as the risks of unforeseen and undesirable effects on protein therapeutics. Furthermore, the perils of production site closure due to undetected cell line contaminants (e.g. mycoplasma or adventitious viral agents) would be reduced by implementing a more efficient cell characterization process within the operating procedures.

All characterized mammalian cell lines and organisms contain remnants of past viral, including retroviral, infections, resulting in viral genes being integrated into the cell genome. These viral remnants are often crippled and mutated, but some of them nevertheless remain expressed and lead to the generation of viral-like particles that can be detected using e.g. electron microscopy.

CHO cells are most widely used, as they can be stable hosts for the expression of heterologous genes, have a long history of safety, produce proteins that bear human-like post-translational modification, and are relatively simple to adapt to adherence-independent and rapid growth in serum-free synthetic media in bioreactors. Nevertheless, the development and documentation of highly efficient clonal cell lines for cultures in bioreactors is often slow and labor-intensive, still suffering from a series of bottlenecks and low protein yield.

CHO cells are considered safe for therapeutic protein production relative to other cell lines. Nevertheless, they also contain remnants of infectious viral particles that require regulatory documentation, as these remnants lead to the release of viral particles by CHO cells (Lie, Y. S. et al., 1994). Furthermore, integrated proviral genomes may potentially recombine, mutate and thereby change properties, albeit at a low frequency.

CHO cells whose genome is depleted of expressed viral remnants, and thereby provide a safer mammalian cell host for therapeutic protein production, are highly desirable.

The recent development of high-throughput DNA sequencing (Next-Generation DNA Sequencing or NGS) based techniques to characterize the genome of cells used to produce proteins (e.g. CHO cells) has led to the successful characterization of transgene integrations sites (Kostyrko et al, 2016).

The publications and other materials, including patents and patent applications, used herein to illustrate the invention and, in particular, to provide additional details respecting the practice are incorporated by reference. For convenience, the publications are referenced in the following text by author and date and are listed alphabetically by author in the appended bibliography.

Given the continued detection of adventitious agents within cells used at therapeutic production sites, pressure is mounting from regulatory agencies such as the FDA to use production cell lines that are better characterized genetically. The FDA defines adventitious agents and includes retroviruses integrated into the genome of a cell (see the FDA's Guidance for Industry Characterization and Qualification of Cell Substrates and Other Biological Materials Used in the Production of Viral Vaccines for Infectious Disease Indications, February 2010). However, currently the analytical and bio-informatics tools required for the complete assembly of the genome of the various CHO cell sublines and for the analysis and interpretation of the genome sequences are often lacking.

### DESCRIPTION OF THE FIGURES

FIGS. 1a and 1b show the genesis of an endogenous retrovirus (ERV) (a) and its exit after dormant existence within the host genome (b). A multiplication of the retrovirus requires integration of genes encoding reverse transcriptase and other components of the virus cycle. ERVs are almost exclusively found in the animal kingdom.

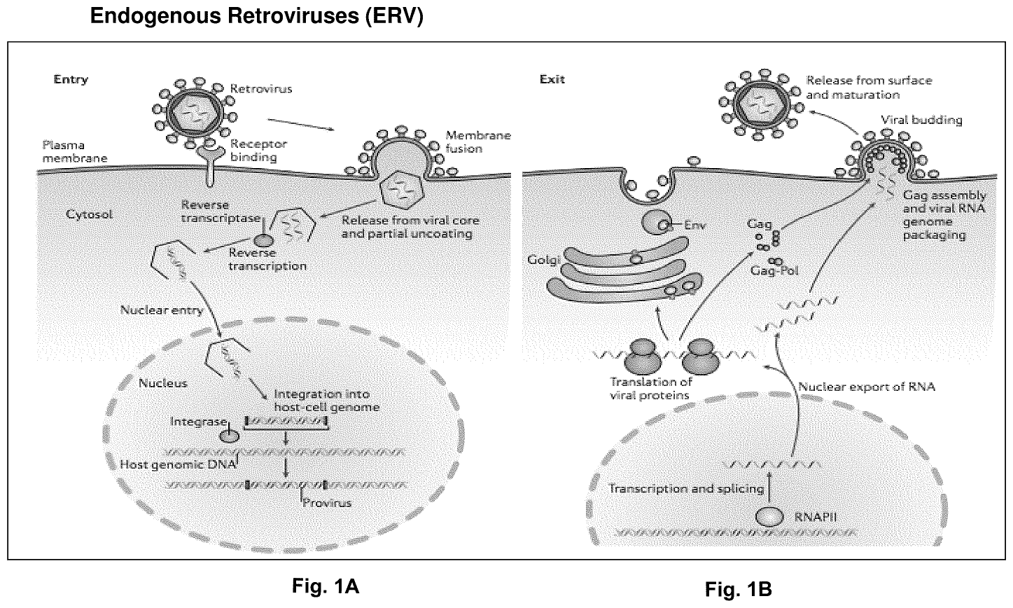

FIG. 2A shows the components of the ESCRT (endosomal sorting complexes required for transport) complex (comprising cytosolic protein complexes, known as ESCRT-0, ESCRT-I, ESCRT-II, and ESCRT-III and a number of accessory proteins including ALIX) and the interaction of the viral p6 protein therewith resulting in the release of viral vesicles.

FIG. 2B shows different amino acid motives of ERVs allowing budding (“budding motives) that were determined via hidden Markov modeling (HMM) from the translated gag (group specific antigen) open reading frame (ORF).

FIG. 3 shows ways of determining budding motives (see FIG. 1C) via HMM (Hidden Markov model) in pp12 encoded by the gag region. Based on the PPXY motif known to be common to gammaretroviruses, the overlapping PPYP motif from Group 1 and Group 2 CHO cell ERVs was determined and served as a target for mutagenesis. Also shown are the number of the first base pair of the compared sequences as listed.

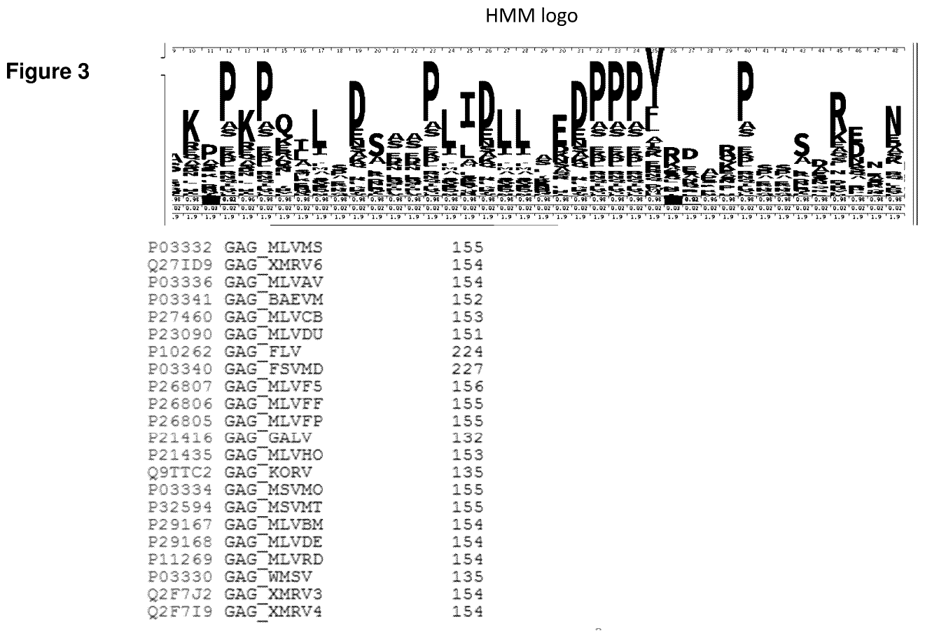

FIG. 4A shows schematically how ERV elements can be found in the genome of a cell based on a comparison of sequence of the cellular genome of the CHO-K1 cell-derived cell line termed CHO-M (from the PacBio® sequencing and genome assembly work) with sequences from typical retroviral elements (gag, pol, env, LTRs (long terminal repeats)). 77 hits were obtained from our PacBio® CHO-M cell genome. The hits were selected for further investigation and determination of consensus sequences.

FIG. 4B shows different steps in further defining and analyzing the consensus sequences and comparisons to the published Chinese hamster genome.

FIG. 4C shows the experimental validation of specific of gammaretrovirus ERV-like sequences identified in the CHO-M cells' genome. The validation of ERVs was performed using PCR primers hybridizing to the CHO genome sequence on either side of the predicted integrated viral genome (validation of gammaretrovirus type C sequences found using bioinformatics analyses). On a gel, the larger sequences (upper three circles) are alleles with integrated genome, while the alleles without viral integration move faster on the gel (lower three circles).

FIG. 5 shows how potentially dangerous ERVs, ergo ERVs that might be awoken, e.g. by cellular/genomic stress, can be determined. The stress can be applied at different times, in different types of cultures and/or after multiple transfections. Epigenetic data provides indications whether or not an ERV element is transcribed (epigenetic data including iRNA assessment and methylation status). During these stress periods infectious ERVs are more likely to be awoken and can be detected.

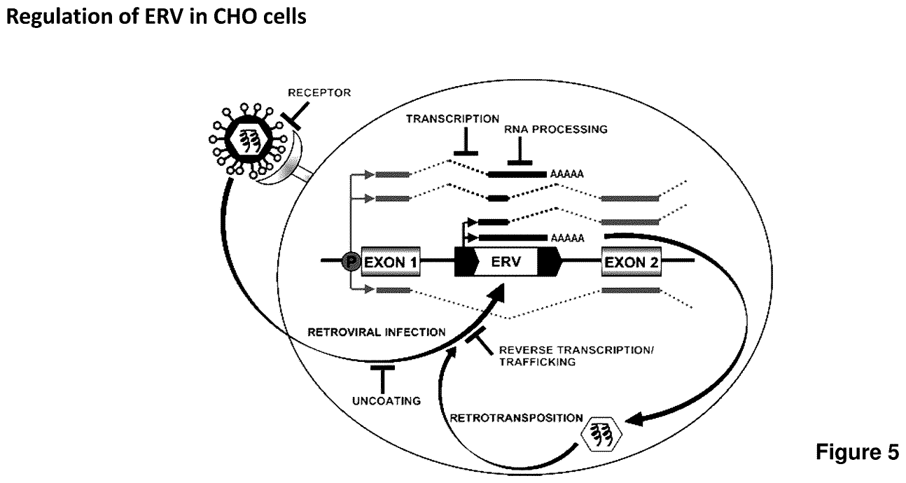

FIG. 6 shows CRISPR/Cas9 systems: using a guide RNA (also called a single guide RNA, or sgRNA), (a) system by Carrol et al. 2013 and (b) system by Guilinger et al. 2014, in which gene targeting (GT) is triggered by double stranded brakes (DSBs), illustrating that nucleases facilitate GT. Site-directed DSBs increase GT up to 10000 fold, but may also trigger unwanted off-target mutations. The nuclease activity is variable between 10-80%.

FIG. 7 shows the three main repair pathways competing for the repair of DSBs, namely, non-homologous end joining (NHEJ), Microhomology mediated end joining (MMEJ) and homologous recombination (HR). The pathway of repair affects the genome editing outcome, which may lead to deletions, gene conversion or to the effective restoration of the original sequence.

FIG. 8 shows the restoration of a functional GFP-coding sequence from a donor plasmid encoding part of GFP in the genome of DG44 CHO cells using the CRISPR/Cas 9 system. Results are shown for CRISPR/Cas 9+donor (a), CRISPR/Cas 9 only (b), and donor only (c). It can be seen that GFP reconstitution requires CRISPR/Cas9 as well as a donor plasmid (about 0.4% of GFP is repaired in the transfected cells in (a)). In the DG44 system, there is no GFP measurable with the CRISPR/Cas9 only or the donor plasmid only.

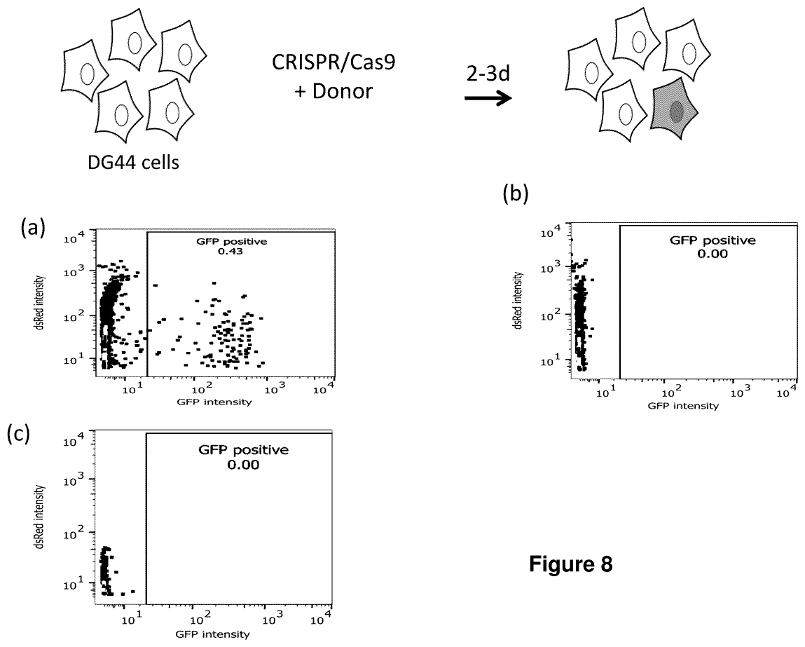

FIG. 9 shows the % of gene correction via the donor plasmid of FIG. 8 (DG44 CHO cells) depending on silencing/knockdown (via siRNAs) of genes influencing different repair pathways. Silencing of genes in the NHEJ pathway increases the % of correction (20% increase upon silencing/knockdown of Ligase 4 and XRCC4). Silencing/knockdown of genes in HDR (homologous directed repair pathways, HR and MMEJ) mostly reduces the % of correction. Silencing of certain genes in HDR increases the % of correction (Nbs4, part of the MRN complex). (The x axis shows the different silencing RNAs used organized according to repair pathway, * indicates significant differences).

FIG. 10 shows the restoration of a functional GFP-coding sequence from a donor plasmid encoding part of GFP in the genome of CHO-M (CHO-K1 variant) cells using the CRISPR/Cas9 system. Transfection with the CRISPR/Cas9 as well as a donor plasmid leads to the 0.7% repaired GFP. The CHO-M cells are thus less recalcitrant to repair than the DG44 cells.

FIG. 11 shows the % of donor plasmid integration of FIG. 10 (CHO-K1 cells) depending on silencing/knockdown (via siRNAs) of genes influencing different repair pathways. Silencing of genes in HDR (homologous directed repair pathways, HR and MMEJ) reduced the % of gene correction. The Figure also shows a high degree of reproducibly of the results from the 3 clones tested. Silencing of Rad51 (via siRad51) and thus the slowdown/shut down of HR as repair pathway lead in all three clones to a significant reduction of GFP positive cells, while slowdown/shut down of the NHEJ via siXrcc4 had essentially no effect on the percentile of GFP positive cells.

FIG. 12A shows the increase or decrease of gene correction from FIG. 10 (CHO-K1 cells) depending on silencing (via siRNAs) of genes influencing different repair pathways. Silencing of genes in HR (homologous repair) partially reduces the % of correction (Rad 51 silencing/knockdown) and partially increases it (Mre11 silencing/knockdown). (The x axis shows the different silencing RNAs used organized according to repair pathway, * indicate significant differences).

FIG. 12B shows the increase of expression of rate limiting HR and/or MMEJ proteins stimulates gene correction in a dose dependent manner. As can be seen an empty vector provides a base level of 1, wherein certain doses of expression vectors providing the rate liming proteins shown increase (upregulate the respective gene) gene correction, by more than 0.05 fold, more than 0.1 fold, 0.2 fold, 0.3 fold, 0.4 fold or 0.5 fold.

FIG. 13 shows the three classes of gammaretroviruses that might be integrated into the genome of the cells to form gammaretrovirus-related ERVs. The figure indicates viruses within each class (I-III) that were found to be present and active when analyzing a CHO genome obtained via PACBIO sequencing, searching for GAL, POL, ENV, LTR sequences (80% coverage, 80% sequence identity): 159 IAP (Intracisternal A-type particles) sequences and 144 type C murine ERV-like sequences were found (boldly circled), as well as 6 sequences related to GALV (Gibbon Ape Leukemia Virus).

FIG. 14 shows a neighbor-joining consensus tree based on 77 GAG/POL/ENV concatenated sequences of gammaretrovisus-like ERVs from the CHO genome. Group 1 and 2 are distinguished as described elsewhere herein.

FIG. 15 shows an alignment of GAG RNAs from a selection of CHO gammaretrovirus-like ERVs transcripts characterized from CHO-M cells. The marks underneath the aligned sequences represent mutations in the GAG open reading frame. Both groups are well conserved and constitute recent integrations in Chinese hamster evolution.

FIG. 16 shows the CpG DNA methylation status of specific ERV elements identified in the CHO-M cells. The 5′LTR ERVs are hypo-methylated in these specific examples, suggesting that they correspond to transcribed ERVs.

FIG. 17 shows a neighbor-joining consensus tree based on 121 GAG sequences of the gammaretrovirus-like ERV from a CHO genome. Both group 1 and 2 contain transcriptionally active ERVs. The circled sequence in group 2 was found to be active, but contained stop codons, the multiple circled sequences in group 1 were found to be active and not to contain a stop codon in the coding sequence. A Gag and Pol cDNA analysis was consistent with the existence of functional ERVs. Potentially expressed ERV are framed with a black rectangle. Based on those sequences, a consensus sequence of group 1 viruses was determined for CRISPR/Cas9 processing.

FIG. 18 shows the strategy used for CRISPR/Cas9 mediated ERV inactivation of the sequences from FIG. 17, with either the PPYP motif and the myristoylation motif as a target (both interfering with ERV budding). The arrows show where double stranded breaks were made. For sgRNA design, see Material and Methods.

FIG. 19 shows two experimental set-ups for ERV inactivation, one without and one with inhibition of HDR repair pathways, here via Rad51 siRNA. ERV disruption is validated via mRNA (i.e. cDNA), as assayed by endpoint PCR, or by sequencing to determine the reading frame.

FIG. 20 shows the effect of the transfection of three individual Rad51-targeting siRNAs on the Rad51 mRNA level and on the homologous recombination repair activity relative to the levels of cells treated with non-specific negative-control siRNAs. Square: Effect of the transfection of the mix (siRad51 3x) of the three Rad51-targeting siRNAs on the Rad51 mRNA level (x-axis) and on the homologous recombination repair activity (y-axis), relative to the levels of cells treated with non-specific negative-control siRNAs which was set to a value of 1. Closed circles: similar assays performed with each of the three siRNA used individually (siRad51_1, siRad51_2, siRad51_3), to demonstrate similar effects on Rad51 and HR inhibition as the mix of the three siRNAs. As can be seen, Rad51 siRNAs are efficient and specific.

FIG. 21 shows effects of CRISPR-Cas9 when used in conjunction to specific sgRNAs in a cDNA PCR assay, which shows reduced ERV expression with ERV-specific primers: The first and second CRISPR experiments show reproducibility and the third one shows the effect of an additional RAD51 siRNA mediated inhibition prior to the CRISPR-Cas9 treatment. Strong ERV mRNA signal losses with PPYP5 sgRNA as well as with RAD51 knockdowns were observed (gel on the top). In the graph at the bottom, the ERV group 1 consensus sequence is shown at the top (SEQ ID NO. 30), a control (untreated cells) Group 1 PPYP motif sequence is shown in the middle (SEQ ID NO. 31) and an example of an out-of-frame deletion obtained from PPYP7 sgRNA treated cells is shown at the bottom (SEQ ID NO. 32).

FIG. 22 shows the sequencing electropherograms of amplified GAG ERV sequences, from untreated CHO cells (CHO WT), or from CHO cells treated with the PPYP6 sgRNA-programed Cas9 nuclease, as obtained from the first CRISPR experiment (sample 1) and the second (sample 2) CRISPR experiment, as depicted in FIG. 21. The Cas9 cleavage site is indicated by the vertical line, after which a mixture of sequences is observed for the treated cells, showing that 50% or more of the GAG sequences were mutated.

### SUMMARY OF THE INVENTION

The present invention is, in one embodiment, directed at an engineered cell, preferably of a mammalian cell line such as an engineered CHO cell, including an engineered CHO-K1 such as a CHO-M cell, comprising:

a genome of the cell, wherein the genome comprises one or more alterations comprising:
deletions,
additions/integrations, and/or
substitutions,
   of one or more nucleic acids in one or more, generally, more than 10, 20, 30, 40, 50, 60, 80, 90 or 100 endogenous retrovirus (ERV) elements which are part of said genome.

The ERV elements may be from gamma retroviral ERVs, including Koala epidemic viral (KoRV), Mouse Mammary Tumor Viral (MMTV), Mouse Leukemia Viral (MLV) ERVs. The one or more alterations may preferably suppress or eliminate release of or be adapted to suppress or eliminate one or more, preferably more than 60%, 70%, 80%, 90%, 95% or 100% of said ERVs.

The one or more altered (ERV) elements may be or may be from a gag, pol and/or env gene, preferably from the gag gene, encoding, a MA (matrix), CA (capsid), NC (nucleocapsid), a further domain encoding proteins such as pp12 or p6 and/or may be long terminal repeats (LTRs) of a ERV.

The one or more ERV elements may in particular encode a Gag (groups antigen) protein, a Pol (reverse transcriptase) protein and/or an Env (envelope) protein.

The additions/insertions, deletions or substitutions may comprise more than 2, 3, 4, 5, 10, 15, 20, 25, 30, 40, 45, 50, 55, 60, 65, 70, 75, 80, 85, 90, 95, 100, 150 or 200 nucleic acids.

The alterations may be targeted to the one or more (ERV) elements, which may be or may be from one or more gag, pol and/or env genes, preferably gag genes, and sequences of the gene may be subjected to the targeted integrations of, e.g., a transgene encoding a marker protein such as GFP (green fluorescent protein), or to amino-acid substitutions leading to a dominant-negative phenotype.

The ERV elements may are from a gag gene and at least 1, 2, 3, 4, 5, 6, 7, 8, 9, 10 or more or 10%, 20%, 30%, 40%, 50%, 60%, 70%, 80%, 90%, 95%, 98%, 99% or all of the elements may comprise said alterations.

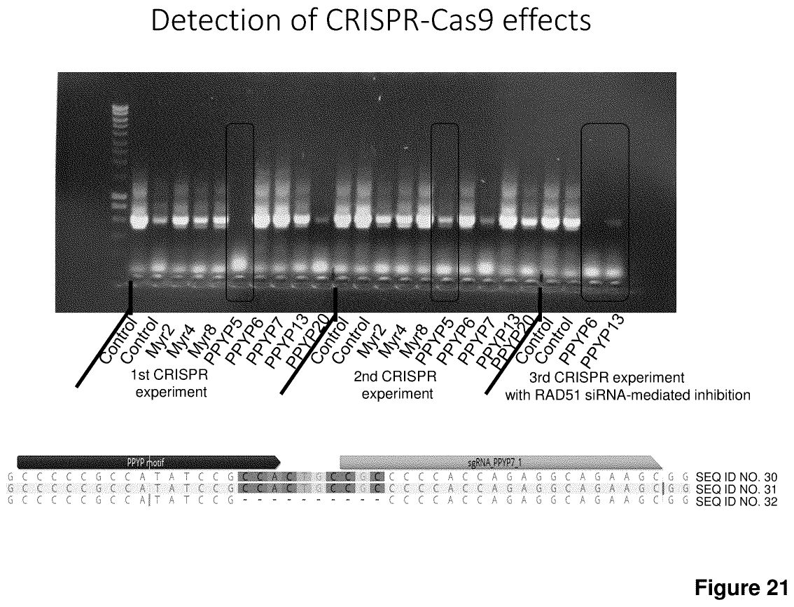

The alterations may be in consensus sequences of the EVR elements, and the consensus sequence may be at least 5, 10, 15, 20, 25, 30, 35, 40, 45, 50 base pairs long, preferably between 15 and 25 or between 30 and 50 base pairs long (compare FIG. 21B).

The engineered cell may release no or substantially no ERVs.

The cell may also comprise heterologous nucleic acid sequences encoding, inhibiting or activating one or more proteins of a recombination pathway, and/or heterologous nucleic acid sequences encoding one or more sequences or proteins suppressing expression of the one or more proteins of a recombination pathway, preferably Nbs1, Mre11, Rad51, Ligase 1 and/or Ligase 3.

The heterologous nucleic acid sequences may be present as a vector/expressed by a vector that is preferably not integrated into the genome of the cell.

The engineered cell may also further comprise heterologous donor DNA, preferably on a vector, encoding one or more marker proteins such as GFP (green fluorescent protein).

The engineered cell may further comprise a transgene. The transgene may be a marker gene encoding a marker protein such as GFP and/or a biotherapeutic which may preferably be expressed from a vector integrated into the genome.

The one or more alterations may be or comprise the substitution of one or more myristoylated amino acids within a myristoylation motif in one or more of the ERV elements, such as a gag gene or an ERV element being from the gag gene, with a non-myristoylated amino acid.

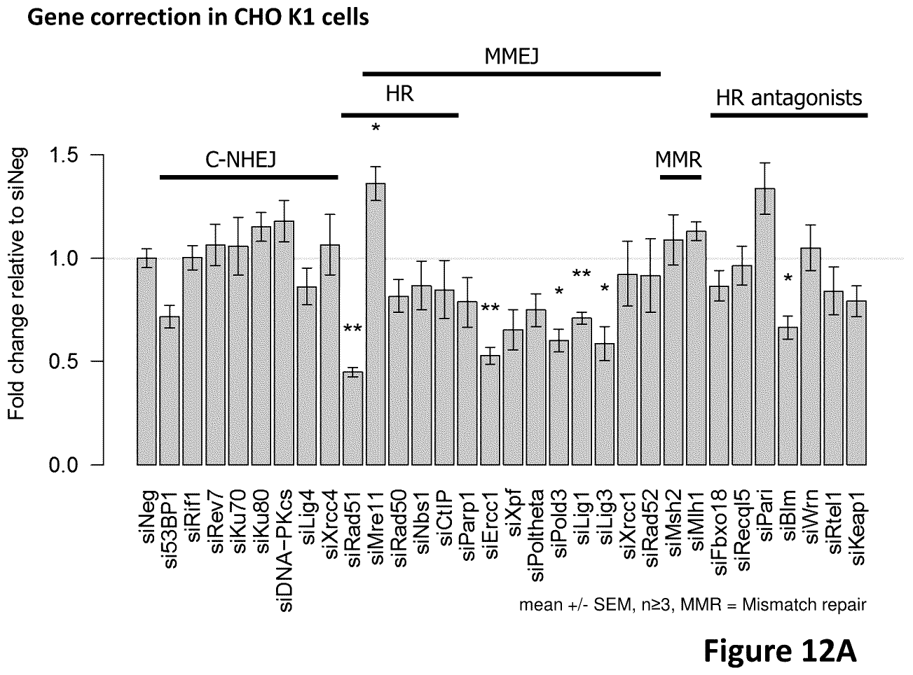

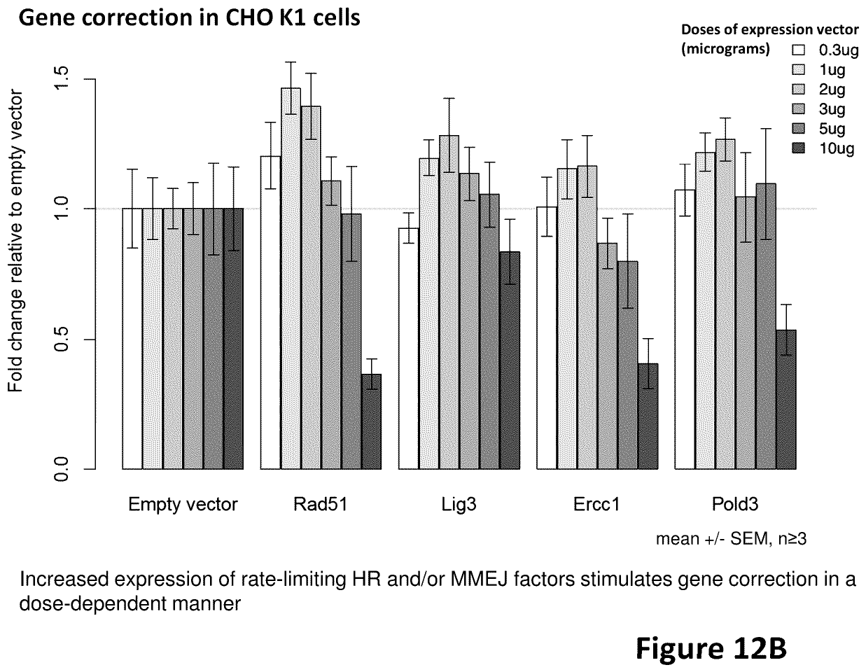

For this purpose, the cell is modified to increase HR/MMEJ expression (see FIGS. 12A and 12B), by providing/expressing in said cell at least transiently sequences, such as in form of a transiently expressed vector encoding HR and/or MMEJ proteins such as Pad51, Lig3, Ercc1, Pold3; and/or sequences, such as siRNA sequences, such as siMRe11 or vectors encoding the same.

The ERV element(s) such as a gag gene or an ERV element being from the gag gene, may comprise a PPYP motif and (i) sequences encoding the PPYP motif and/or sequences of up to 20 or 30 nucleic acids flanking the sequences in (i) may comprise the alternation.

The engineered cell may be a CHO cell and may have one or more deletions, additions, and/or substitutions in one or more SEQ IDs 1, 2, 3, 4 or sequences having more than 90% or 95% sequence identity therewith, preferably within the ERV elements of said sequences.

The engineered cell may be a CHO cell and may have one or more deletions, additions, and/or substitutions in ERV group 1 consensus sequence, such as SEQ ID NO. 30 or in sequences having more than 90% sequence identify therewith and wherein the sequences with the deletions, additions, and/or substitution no longer encode a functional Gag protein.

The present invention is also directed to a method for improved genome editing comprising:
(i) providing or introducing a non-naturally occurring/heterologous system for creating single or double stranded brakes in a target nucleic acid sequence of a cell, preferably a cell of interest, such as a consensus sequence of a ERV element, preferably a ERV element that is or is from a gag gene, and further
(ii) providing or introducing into the cell
(a) heterologous nucleic acid sequences encoding or activating one or more proteins of one or more of the recombination pathways, and/or
(b) heterologous sequences encoding one or more sequences, such as siRNAs or proteins, suppressing/decreasing the expression of the one or more proteins of one or more of the recombination pathways, wherein said heterologous system of (i) and/or sequences of (ii) (a) and/or (ii) (b) are preferably transiently expressed in the cell of interest.
The heterologous sequences may encode, activate or inactivate one or more proteins of a recombination pathway, in particular one or more proteins of Homologous Recombination (HR), pathway, in particular Rad51, Nbs1, Mre1 1, Ligase 1 and/or Ligase 3.
A marker gene, such as GFP, may be inserted into the target nucleic acid sequence via homologous recombination. Cells comprising the marker gene may be preferably selected.
The heterologous sequences of (a) above, may encode, activate or inactivate proteins of the MNR complex, such as Nbs1 and/or Mre 11, and/or said heterologous sequences of (b) may encode sequences or proteins suppressing expression of one or more proteins of Homologous Recombination (HR) pathway such as Rad51, Nbs1, Mre11, Ligase 1 and/or Ligase 3.
A deletion or insertion may be introduced into the one or more ERV elements via non-homologous end-joining (NHEJ) or microhomology mediated end joining (MMEJ).
The heterologous sequences may be part of- or expressed from integrating or preferably non-integrating vectors.
The non-naturally occurring/heterologous system for introducing single or double stranded brakes into a target nucleic acid sequence may be a CRISPR/Cas9 system or may be based thereon.
One or more myristoylated amino acids within a myristoylation motif in one or more of the ERV elements may be substituted with a non-myristoylated amino acid.
Sequences encoding a PPYP motif and/or sequences of up to 10, 20 or 30 nucleic acids flanking the motif may comprise an alteration.
Sequences suppressing expression of, e.g., HR proteins such as Rad51 may be only transiently expressed in the cells.
The invention is also directed an engineered cell preferably a mammalian cell such as an engineered CHO cell, including a CHO-K1 cell, which preferably does not release viral particles/releases substantially no viral particles, especially under standard and/or stressing culturing condition, and wherein a genome of the cell of comprises one or more alterations comprising:
(a) deletions,
(b) additions/integrations, and/or
(c) substitutions,
   of one or more nucleic acids in one or more, generally, more than 10, 20, 30, 40, 50, 60, 80, 90 or 100 endogenous retrovirus (ERV) elements, and is preferably produced by any one of the methods of set forth herein.

The present invention is also directed to a kit comprising:
(i) in one container at least one non-integrating vector encoding at least a nuclease, such as CRISPR,
(ii) in the same or a further container one or more guide RNAs or sequences encoding the one or more guide RNAs targeting a motif in a ERV element
(iii) in same or a further container one or more siRNAs or sequences encoding one or more siRNAs, and
in a further container instruction of how to provide (i), (ii) and (iii) within a cell.

The sequences encoding the one or more guide RNAs targeting the motif in the ERV element and/or the sequences encoding one or more siRNAs may be part of a vector. Any of the vectors described herein may be only transiently expressed and/or may be a non-integrating vector of (i). The ERV element may encode a Gag protein. The motif may be a myristoylation motif or a PPXY motif, in particular a PPYP motif. The siRNA(s) may be directed against a gene of the HR pathway.

### DESCRIPTION OF VARIOUS AND PREFERRED EMBODIMENTS OF THE INVENTION

A cell, preferably a mammalian cell/eukaryotic cell, that according to the present invention is engineered to produce an engineered cell is capable of being maintained under cell culture conditions. Standard cell culture conditions are from 30 to 40° C., preferably at or at about 37° C., for instance in fully synthetic culture medium as used in the production of recombinant proteins. Non-limiting examples of this type of cell are non-primate eukaryotic cells such as Chinese hamster ovary (CHOs) cells including the CHO-K1 (ATCC CCL 61) cells and SURE CHO-M cells (derivative of CHO-K1), and baby hamster kidney cells (BHK, ATCC CCL 10). Primate eukaryotic host cells include, e.g., human cervical carcinoma cells (HELA, ATCC CCL 2) and 293 [ATCC CRL 1573] as well as 3T3 [ATCC CCL 163] and monkey kidney CV1 line [ATCC CCL 70], also transformed with SV40 (COS-7, ATCC CRL-1587). The term engineered signifies a cell that has been altered, e.g., by transfection with, e.g., a transgenic sequence and/or by mutation. As the person skilled in the art will readily understand these cells are, even prior to engineering as described herein, non-naturally occurring cells. The above-mentioned cells, in particular, the various CHO cells, are commonly used in biotechnological applications, such as for the production of therapeutic proteins, and are herein referred to as cells of interest. As the person skilled in the art will readily understand, other cells than the ones mentioned above might be cells of interest as long as they are used in biotechnological applications, in particular for the expression of, e.g., therapeutic proteins.

The three major proteins encoded within the retroviral genome are Gag, Pol, and Env. Gag (Group Antigens) encoded by the gag gene is a polyprotein, which is processed to matrix and other core proteins, including the nucleoprotein core particle, that determine the retroviral core. Pol is the reverse transcriptase, encoded by the pol gene and has RNase H and integrase function. Its activity results in the double-stranded DNA pre-integrated form of the virus and, via the integrase function, for the integration into the host genome, and also via the RNase function, the reverse transcription after integration into the genome of the host. Env is the envelope protein, encoded by the env gene, and resides in the lipid layer of the virus determining the viral tropism.

Endogenous retroviruses (ERVs), such as viruses from the genus of gamaretroviruses, including Koala epidemic virus (KoRV), Mouse Mammary Tumor Virus (MMTV), Mouse Leukemia Virus (MLV) are adventitious agents that occur in any animal genome including, e.g., in hamster genomes and genomes of related species. ERVs are maintained in the genomes and may have certain advantages for the cells into whose genome they are integrated, including providing a source of genetic diversity and protection against other viral pathogens. However, they can become infectious. Cancer and/or epigenetic modifications can increase ERV awakening. Lewis et al. identified 403 possible retrovirus proteins in CHO cells based on a comparison of homologies to a database of 115 viruses. Lewis et al. reported that 40% of their mRNAs were expressed, thereby suggesting that many of these may be still be synthesizing retroviral components (Lewis et al., 2013, Supplement). In the present context, murine retrovirus type C sequences were used as a comparison tool (data obtained from a 1994 publication). There are four species in this group as noted in the Viralzone website (December 2015) hosted by SIB (Swiss Institute of Bioinformatics). They also occur frequently in mouse genome and are known to cause cancer in mice. However, as the person skilled in the art will appreciate, the sequences of many other retroviruses can be used to search for ERVs in cells of interest.

The engineered cell according to the present invention, may comprise a genome that, in most parts, is identical to the genome of the cell it is derived from such as a CHO-K1 cell. However, at least one or more, often many ERV elements which are part of these genomes will contain alterations as described herein.

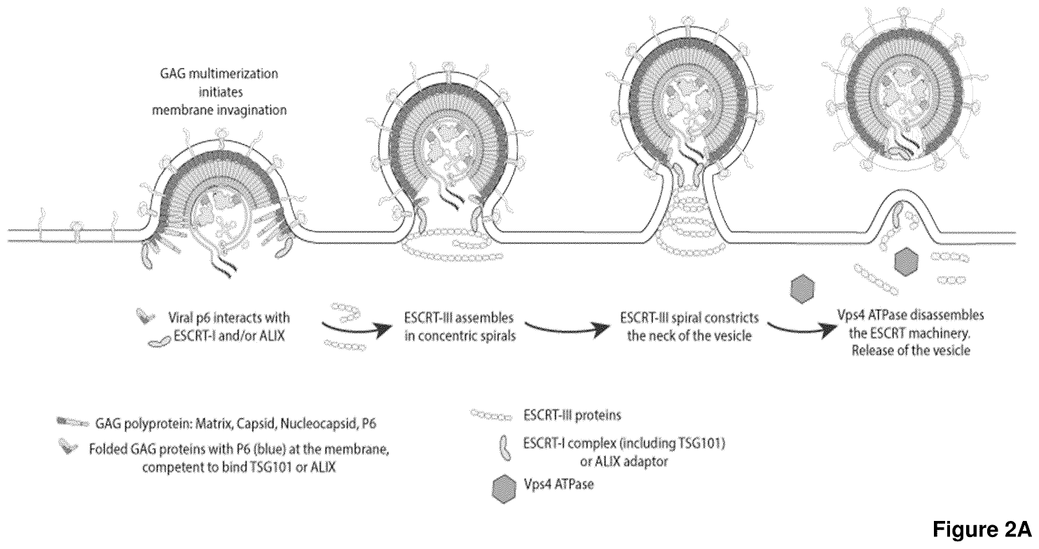

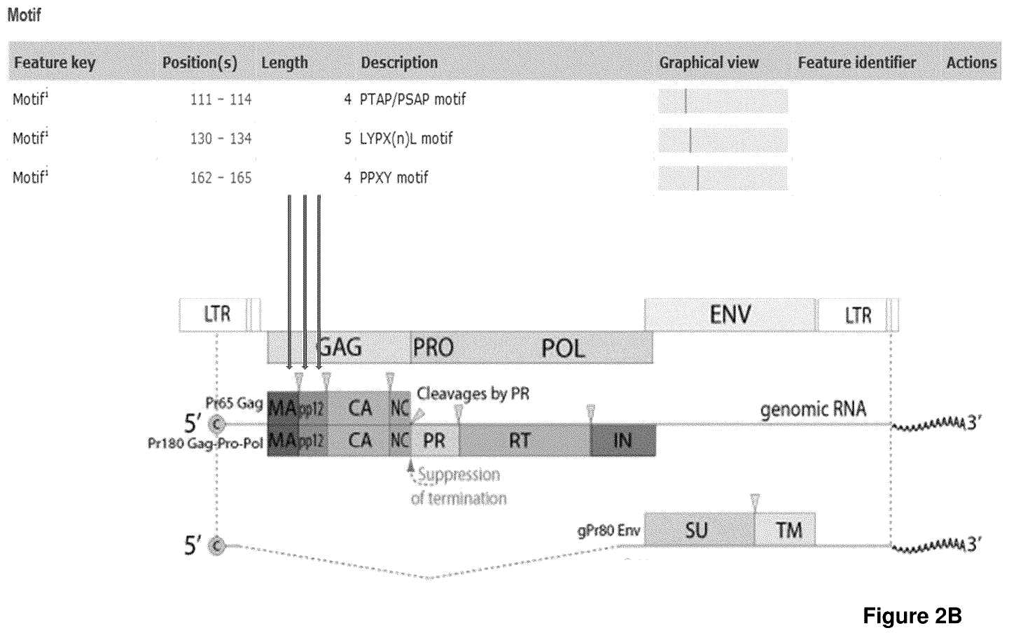

An ERV element according to the present invention describes primarily a section of the ERV nucleic acid sequence that provides in the corresponding non-integrated virus a functional entity. As the person skilled in the art will appreciate, often parts of these sections are disrupted or deleted in the integrated virus DNA. Thus, included in this definition are all genes (gag, pol, env) and, secondarily, parts thereof set forth below and shown in FIG. 2B but also truncated and/or modified versions thereof that are present in the genome of a cell. ERV elements are referred to as being, e.g., from gag, if the gag gene is their source, but they do not necessarily encode a full functional Gag protein. ERV elements that are said to encode the Gag protein, encode a functional Gag protein.

The gag gene gives rise to a Gag precursor protein, which is expressed from the unspliced viral mRNA. The Gag precursor protein is cleaved by the virally encoded protease (a product of the pol gene) during the process of viral maturation into generally four smaller proteins designated MA (matrix), CA (capsid), NC (nucleocapsid), and a further protein domain (e.g. pp12 in murine leukemia virus or p6 in HIV).

The MA polypeptide is derived from the N-terminal, myristoylated end of the precursor protein. Most MA molecules remain attached to the inner surface of the virion lipid bilayer, stabilizing the particle.

The CA protein forms the conical core of viral particles.

The NC region of Gag is responsible for specifically recognizing the so-called packaging signal of the retrovirus. The packaging signal comprises four stem loop structures located near the 5′ end of the viral RNA, and is sufficient to mediate the incorporation of a heterologous RNA into virions. NC binds to the packaging signal through interactions mediated by two zinc-finger motifs.

Another protein domain mediates interactions between precursor protein Gag and the accessory protein Vpr, leads to the incorporation of Vpr into assembling virions. The p6 region in HIV also contains a so-called late domain which is required for the efficient release of budding virions from an infected cell. (Hope & Trono, 2000).

The viral protease (Pro), integrase (IN), RNase H, and reverse transcriptase (RT) are expressed within the context of a Gag-Pol fusion protein. The Gag-Pol precursor is generally generated by a ribosomal frame shifting event, which is triggered by a specific cis-acting RNA motif (a heptanucleotide sequence followed by a short stem loop in the distal region of the Gag RNA). When ribosomes encounter this motif, they shift approximately 5% of the time to the pol reading frame without interrupting translation. The frequency of ribosomal frameshifting explains why the Gag and the Gag-Pol precursor are produced at a ratio of approximately 20:1.

During viral maturation, the virally encoded protease cleaves the Pol polypeptide away from Gag and further digests it to separate the protease, RT, RNase H, and integrase activities. These cleavages do not all occur efficiently, for example, roughly 50% of the RT protein remains linked to RNase H as a single polypeptide (p65) (Hope & Trono, 2000).

The pol gene encodes the reverse transcriptase. During the process of reverse transcription, the polymerase makes a double-stranded DNA copy of the dimer of single-stranded genomic RNA present in the virion. RNase H removes the original RNA template from the first DNA strand, allowing synthesis of the complementary strand of DNA. The predominant functional species of the polymerase is a heterodimer. All of the pol gene products can be found within the capsid of released virions.

The IN protein mediates the insertion of the proviral DNA into the genomic DNA of an infected cell. This process is mediated by three distinct functions of IN.

The Env protein is expressed from singly spliced mRNA. First synthesized in the endoplasmic reticulum, Env migrates through the Golgi complex where it undergoes glycosylation. Env glycosylation is generally required for infectivity. A cellular protease cleaves the protein into a transmembrane domain and a surface domain. (Hope & Trono, 2000).

Some ERVs of a genome are released from the cells in the form of virus-like particles and others are not. However, generally the ones that are released have a higher potential to become infectious. Thus, it is generally advantageous to have cells engineered, as described herein, that can express and release no or substantially no ERVs, preferably under standard or stressful culturing conditions. Substantially no ERVs are released if a cell culture comprising the so engineered cell releases less than 50%, less than 40%, less than 30%, less than 20%, less than 10%, preferably 5% of ERVs than their counterpart that has not been subjected to the ERV release reducing procedures described herein. Such a counterpart would, e.g., be a commercially available CHO-K1 cell. No or substantially no expression means that less than 50%, less than 40%, less than 30%, less than 20%, less than 10%, preferably 5%, unmutated Gag mRNA sequence can be detected by PCR and sequencing analysis. No release means that no or substantially no detectable viral sequence release occurs as assessed via a cDNA PCR assay, as shown e.g. in FIG. 21 or as obtained from QIAGEN, QuantiTect Rev. Transcription Kit®. As the person skilled in the art will also appreciate, cells that have been engineered to comprise nucleic acid sequence alterations that inactive protein/peptide production in one, two, three, four, 5, 6, 7, 8, 9, 10 ERV elements can also be advantageous and are part of the present invention. In fact, in certain embodiments it is advantageous to alter one or more ERV elements which is part of the genome of a cell of interest only by one or more mutations to maintain, where appropriate, positive roles that the ERVs might play within the cell. However, as discussed below, certain embodiments of the present invention are particularly suitable for high copy number ERVs (more than 30, 40 or 50 ERVs/per cell) and are able to accomplish that the modified cell does not show any expression of the respective ERV component.

A heterologous nucleic acid sequence is a nucleic acid sequence that does not occur in the cells prior to engineering according to the present invention, while related types of nucleic acid sequences may very well exist in the cell. A transgene as used in the context of the present invention is such a heterologous nucleic acid sequence, in particular a deoxyribonucleotide (DNA) sequence coding for a given mature protein (also referred to herein as a DNA encoding a protein), for a precursor protein or for a functional RNA that does not encode a protein (non-coding RNA). A transgene is isolated and introduced into a cell to produce the transgene product. Some preferred transgenes according to the present invention encode marker proteins such as GFP (green fluorescent protein). Those can be used to detect successful integration into, ergo alternation/inactivation of, ERV elements.

Other transgenes are those that encode, e.g., proteins that shall ultimately be produced by the cell in question such as immunoglobulins (Igs) and Fc-fusion proteins and other proteins, in particular proteins with therapeutical activity (“biotherapeutics”). According to the present invention, additions include integrations such as targeted integrations. However, the person skilled in the art will appreciate that during the integration process certain nucleotides might be lost in the recipient genome. Those integrations are part of the present invention and are considered additions.

As used herein, the term transgene shall, in the context of a DNA encoding a protein, not include untranscribed flanking regions such as RNA transcription initiation signals, promoters or enhancers. Other preferred transgenes include DNA sequences encoding functional RNAs. Thus, the term transgene is used in the present context when referring to a DNA sequence that is introduced into a cell such as a eukaryotic host cell via transfection (which includes in the context of the present invention also transduction, i.e., the introduction via viral vectors) and which encodes a product of interest (“transgene expression product”, e.g., “heterologous protein”). The transgene might be functionally attached to a signal peptide coding sequence, which encodes a signal peptide which in turn mediates and/or facilitates translocation and/or secretion across the endoplasmic reticulum and/or cytoplasmic membrane and is removed prior or during secretion.

A promoter sequence or just promoter is a nucleic acid sequence which is recognized by a host cell for expression of one or more nucleic acid sequences. The promoter sequence contains transcriptional control sequences which regulate the expression of the polynucleotide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell. Promoters according to the present invention include inducible and non-inducible promoters. A nucleic acid sequence is under control of a promoter is the promoter exercises its function on said nucleic acid. The cells/vectors of the present inventions often contain such promoters.

Nucleic acid sequence alterations are alterations, such as additions/insertions, deletions and/or substitutions that do not occur in the cells prior to engineering according to the present invention.

As used herein, “genome editing” refers to the modification (“editing”) of genomic sequences and may comprise a deletion of at least one nucleotide, an addition/insertion of at least one nucleotide, or a substitution of at least one nucleotide. The genomic sequence edited is referred to herein as target nucleic acid sequence. Targeted insertions are insertions that occur at a specific predetermined target site. Genome editing tools introduce double or single stranded breaks into the genome, e.g., via nucleases or nickases, and rely at least in part on the cellular recombination mechanisms (see discussion below) to repair these breaks. These tools also contain generally sequence specific DNA binding modules. ZFNs (Zinc-Finger Nucleases) and TALENs (transcription activator-like effector nucleases) enable a broad range of genetic modifications by inducing DNA double-strand breaks that stimulate error-prone non-homologous end joining (NHEJ) or homology-directed repair (HDR) at specific genomic locations.

The sequence specificity of CRISPR (clustered, regularly interspaced, short palindromic repeats) systems is determined by small RNAs. CRISPR loci are composed of a series of repeats separated by ‘spacer’ sequences that match the genomes of bacteriophages and other mobile genetic elements. The repeat-spacer array is transcribed as a long precursor and processed within repeat sequences to generate small crRNA that specify the target sequences (also known as protospacers) cleaved by CRISPR systems. For cleavage, the presence of a sequence motif immediately downstream of the target region is often required, known as the protospacer-adjacent motif (PAM). CRISPR-associated (cas) genes usually flank the repeat-spacer array and encode the enzymatic machinery responsible for crRNA (CRISPR RNA) biogenesis and targeting. Cas9 is a dsDNA endonuclease that uses a crRNA guide to specify the site of cleavage. Loading of the crRNA guide onto Cas9 occurs during the processing of the crRNA precursor and requires a small RNA antisense to the precursor, the tracrRNA, and RNAse III. In contrast to genome editing with ZFNs or TALENs, changing Cas9 target specificity does not require protein engineering but only the design of the short crRNA guide, also termed sgRNA.

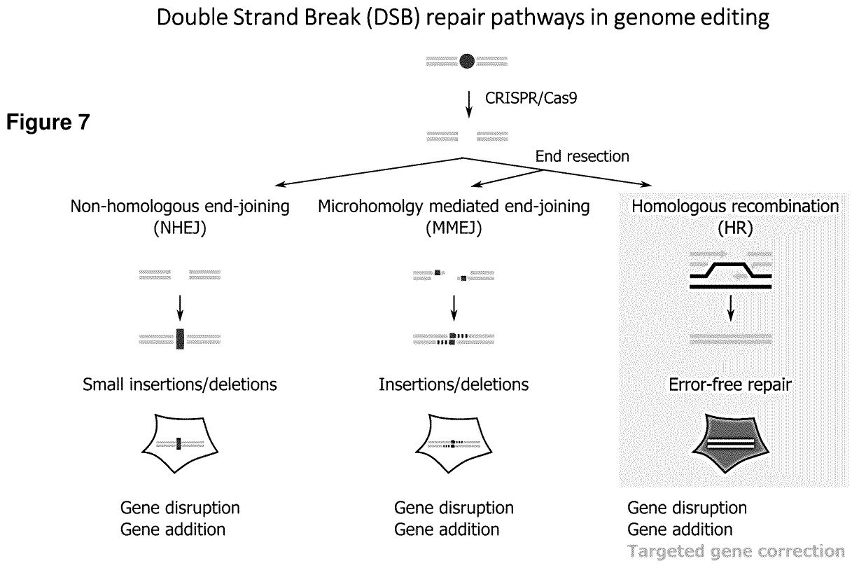

To date, three different variants of the Cas9 nuclease have been adopted in genome-editing protocols. The first is wild-type Cas9, which can site-specifically cleave double-stranded DNA, resulting in the activation of the doublestrand break (DSB) repair machinery. DSBs can be repaired by the cellular Non-Homologous End Joining (NHEJ) pathway, resulting in insertions and/or deletions (indels) which disrupt the targeted locus. Alternatively, if a donor template (see FIG. 7) with homology to the targeted locus is supplied, the DSB may be repaired by the homology-directed repair (HDR) pathway allowing for precise replacement mutations to be made.

The Cas9 system was further engineered towards increased precision by developing a mutant form, known as Cas9D10A, with only nickase activity. This means it cleaves only one DNA strand, and does not activate NHEJ. Instead, when provided with a homologous repair template, DNA repairs are conducted via the high-fidelity HDR pathway only, resulting in reduced indel mutations. Cas9D10A is therefore in many applications more appealing in terms of target specificity when loci are targeted by paired Cas9 complexes designed to generate adjacent DNA nicks.

In the context of the present invention, a specific sequence or a consensus sequence of ERV elements are determined to specify the site of cleavage via, e.g., one of the systems above. Such a specific or consensus sequence is preferably between 5 and 50 base pairs long, preferably between 10 and 40 50 or between 15 and 25 or between 25 and 50 or 30 and 50. The consensus sequences may contain, e.g., 1, 2, 3, 4 or 5 mismatches (have more than 60%, 70%, 80%, 90% or 95% complementarity relative to each other), as long as cleave can still be performed. See, e.g, FIG. 21. The above systems are called non-naturally occurring systems or heterologous systems, which means that they are introduced to the cell rather than being a part of the cell prior to engineering according to the present invention.

A vector according to the present invention is a nucleic acid molecule capable of transporting another nucleic acid, such as a transgene that is to be expressed by this vector, to which it has been linked, generally into which it has been integrated. For example, a plasmid is a type of vector, a retrovirus or lentivirus is another type of vector. In a preferred embodiment of the invention, the vector is linearized prior to transfection. An expression vector comprises heterologous regulatory elements or is under the control of such regulatory elements that are designed to further the transcription and/or expression of a nucleic acid sequence, such as a transgene, carried by the expression vector. Regulatory elements comprise enhancers and/or promoters, but also a variety of other elements described herein. Among non-viral vectors, transposons are particularly attractive because of their ability to integrate single copies of DNA sequences with high frequency at multiple loci within the host genome (integrating vector). Unlike viral vectors, some transposons were reported not to integrate preferentially close to cellular genes, and they are thus less likely to introduce deleterious mutations. Moreover, transposons are readily produced and handled, comprising generally of a transposon donor vector containing the cargo DNA flanked by inverted repeat sequences and of a transposase-expressing helper plasmid or mRNA. Several transposon systems were developed to mobilize DNA in a variety of cell lines without interfering with endogenous transposon copies. For instance, the PiggyBac (PB) transposon originally isolated from the cabbage looper moth efficiently transposes cargo DNA into a variety of mammalian cells.

In the context of the present invention, vectors, in particular non-integrating vectors, may also be used for transient expression of a gene or a functional RNA. Transient expression is an expression for a limited amount of time and the time period of expression depends on the vector design and culturing conditions. However, transient expression means expression over a period of at least 24 hours but generally not more than 7 days.

Epigenetic regulatory elements can be used to protect the cargo DNA from unwanted epigenetic effects when placed near the transgene on plasmid vectors. For example, elements called matrix attachment region (MARs) were proposed to increase cargo DNA genomic integration and transcription while preventing heterochromatin silencing, as exemplified by the potent human MAR 1-68. They can also act as insulators and thereby prevent the activation of neighboring cellular genes. MAR elements have thus been used to mediate high and sustained expression in the context of plasmid or viral vectors. For transient gene expression, non-integrating vectors (sometimes referred to as episomal vectors) such as plasmids or non-integrating lentiviral (NIL) vectors may be used. They may be stably or transiently maintained and replicated within the host cell.

The vector sequence of a vector is the DNA or RNA sequence of the vector excluding any “other” nucleic acids such as transgenes as well as genetic elements such as MAR elements.

The term sequence identity refers to a measure of the identity of nucleotide sequences or amino acid sequences. In general, the sequences are aligned so that the highest order match is obtained. “Identity”, per se, has a recognized meaning in the art and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans as defining identical nucleotides or amino acids at a given position in the sequence (Carillo, H. & Lipton, D., SIAM J Applied Math 48:1073 (1988)).

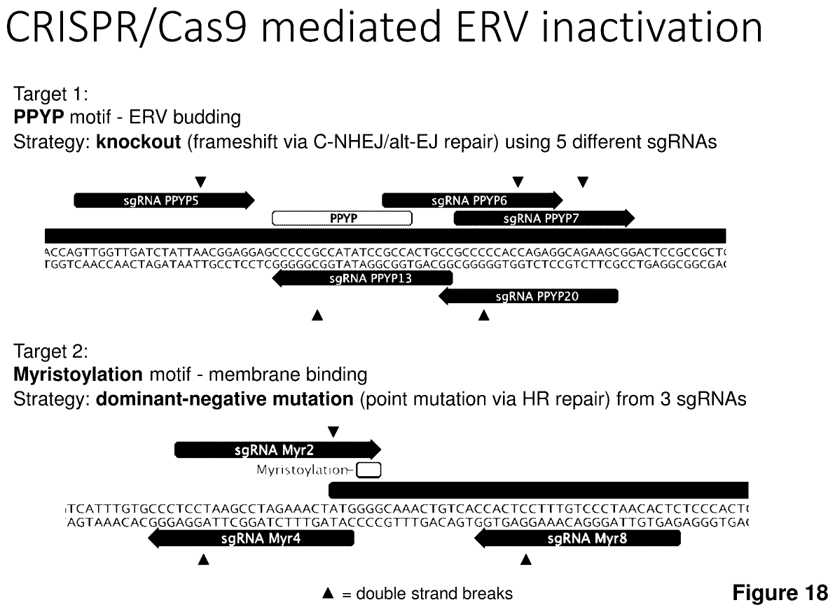

Whether any particular nucleic acid molecule is at least 50%, 60%, 70%, 75%, 80%, 85%, 90%, 95%, 96%, 97%, 98% or 99% identical to, for instance, the gammaretrovirus-like sequences of SEQ ID NOs. 1, 2, 3 or 4, or a part thereof (see, e.g., sequences disclosed in FIG. 18), can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments.

Whether the amino acid sequence is at least 50%, 60%, 70%, 75%, 80%, 85%, 90%, 95%, 96%, 97%, 98% or 99% identical to, for instance a protein expressed by SEQ ID NOs:1, 2, 3 or 4, or a part thereof, can be determined conventionally using known computer programs such the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences.

When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleic acid or amino acid sequence and that gaps in homology of up to 5% of the total number of nucleotides in the reference sequence are allowed.

Another preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

For example, a polynucleotide having 95% “identity” to a reference nucleotide sequence of the present invention, is identical to the reference sequence except that the polynucleotide sequence may include on average up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence, the ORF (open reading frame), or any fragment specified as described herein.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website, together with a description of how to determine sequence identity and sequence similarities using this program.

The present invention includes nucleic acid and/or amino acid molecules having 80%, 85%, 90% or more, 95% or more, 98% sequence identity or complete sequence identity with the sequences disclosed herein and any fragment thereof, in particular fragments spanning up to 40 bps, 30 bps or 20 bps left and right of a PPYP motif.

### THE RECOMBINATION PATHWAYS

The recombination pathways, also known as DNA recombination pathways, are cellular pathways that lead to DNA damage repair, such as the joining of DNA molecule extremities after chromosomal double-strand breaks (DSBs), and to the exchange or fusion of DNA sequences between chromosomal and non-chromosomal DNA molecules, such as e.g. the crossing-over of chromosomes at meiosis or the rearrangement of immunoglobulin genes in lymphocytic cells. The main recombination pathways are the homologous recombination pathway (HR), the non-homologous end-joining pathway (NHEJ) and the microhomology-mediated end-joining (MMEJ) and alternative end-joining (Alt-EJ) pathways.

In the context of the present invention, knock-outs of certain members of one or more of the pathways are often generated via complimentary nucleic acid sequences. A nucleic acid sequence, such as a DNA or RNA, is complimentary to another DNA or RNA, if the nucleotides of, e.g., two single stranded DNA stands or two single stranded RNA strands can form stable hydrogen bonds, such as a hydrogen bond between guanine (G) and cytosine (C). In the cell, complementary base pairing allows, e.g., cells to copy information from one generation to another. In RNA interference (RNAi) complementary base pairing allows, the silencing or complete knock-out of certain target genes. Essentially, siRNA, shRNA or miRNA sequence specifically reduce or knock-out expression of a target gene by having a single RNA strand (e.g. the anti-sense strand in siRNA) align with RNA, in particularly the mRNA of the host cell. The degree of complementarity between two nucleic acid strands may vary, from complete complementarity (each nucleotide is across from its opposite) to partial complementary (50%, 60%, 70%, 80%, 90% or 95%). The degree of complementarity determines the stability of the complex and thus how successfully a gene can be, e.g., knocked-out. Thus, complete or at least 95% complementarity is preferred.

### THE MECHANISMS OF HOMOLOGOUS RECOMBINATION (HR), NHEJ AND MMEJ

### Transgenes Use the Recombination Machineries to Integrate at a Double Strand Break Into the Host Genome.

Double-strand breaks (DSBs) are the biologically most deleterious type of genomic damage potentially leading to cell death or a wide variety of genetic rearrangements. Accurate repair is essential for the successful maintenance and propagation of the genetic information. There are two major DSB repair mechanisms: NHEJ and HR. A third mechanism, called MMEJ often takes effect when the two major DSB repair mechanisms fail. Homologous recombination is a process for genetic exchange between DNA sequences that share homology and is operative predominantly during the S/G2 phases of the cell cycle, while NHEJ simply pieces together two broken DNA ends, usually with no sequence homology, and it functions in all phases of the cell cycle but is of particular importance during G0-G1 and early S-phase of mitotic cells. In vertebrates, HR, NHEJ and MMEJ differentially contribute to DSB repair, depending on the nature of the DSB and the phase of the cell cycle.

### NHEJ: Basic Mechanisms

Conceptually, the molecular mechanism of the NHEJ process seems to be simple: 1) a set of enzymes capture the broken DNA molecule, 2) a molecular bridge that brings the two DNA ends together is formed and 3) the broken molecules are re-ligated. To perform such reactions, the NHEJ machinery in mammalian cells involves two protein complexes, the heterodimer Ku80/Ku70 associated with DNA-PKcs (catalytic subunit of DNA-dependent protein kinase) and DNA ligase IV with its co-factor XRCC4 (X-ray-complementing Chinese hamster gene 4) and many protein factors, such as Artemis and XLF (XRCC4-like factor; or Cernunnos). NHEJ is frequently considered as the error-prone DSB repair because it simply pieces together two broken DNA ends, usually with no sequence homology and it generates small insertions and/or deletions. NHEJ provides a mechanism for the repair of DSBs throughout the cell cycle, but is of particular importance during G0-G1 and early S-phase of mitotic cells. The repair of DSBs by NHEJ is observed in organisms ranging from bacteria to mammals, indicating that it has been conserved during evolution.

After DSB formation the key step in NHEJ repair pathway is the physical juxtaposition of the broken DNA ends. NHEJ is initiated by the association of the Ku70/80 heterodimer protein complex to both ends of the broken DNA molecule to capture, tether the ends together and create a scaffold for the assembly of the other NHEJ key factors. The DNA-bound Ku heterodimer complex recruits DNA-PKcs to the DSB, a 460kDa protein belonging to the PIKK (phosphoinositide 3-kinase-like family of protein kinases) and activates its serine/threonine kinase function. Two DNA-PKcs molecules interact together across the DSB, thus forming a molecular bridge between both broken DNA ends and inhibit their degradation. Then, DNA ends can be directly ligated, although the majority of termini generated from DSB have to be properly processed prior to ligation. Depending of the nature of the break, the action of different combinations of processing enzymes may be required to generate compatible overhangs, by filling gaps, removing damaged DNA or secondary structures surrounding the break. This step in the NHEJ process is considered to be responsible for the occasional loss of nucleotides associated with NHEJ repair. One key end-processing enzyme in mammalian NHEJ is Artemis, a member of the metallo-6-lactamase superfamily of enzymes, which was discovered as the mutated gene in the majority of radiosensitive severe combined immunodeficiency (SCID) patients. Artemis has both a 5′→3′ exonuclease activity and a DNA-PKcs-dependent endonuclease activity towards DNA-containing ds-ss transitions and DNA hairpins (Ma et al., 2002). Its activity is also regulated by ATM. Thus, Artemis seems likely to be involved in multiple DNA-damage responses. However, only a subset of DNA lesions seem to be repaired by Artemis, as no major defect in DSB repair were observed in Artemis-lacking cells.

DNA gaps must be filled in to enable the repair. Addition of nucleotides to a DSB is restricted to polymerases μ and X. By interaction with XRCC4, polynucleotide kinase (PNK) is also recruited to DNA ends to permit both DNA polymerization and ligation. Finally, NHEJ is completed by ligation of the DNA ends, a step carried out by a complex containing XRCC4, DNA ligase IV and XLF. Other ligases can partially substitute DNA ligase IV, because NHEJ can occur in the absence of XRCC4 and Ligase IV. Furthermore, studies showed that XRCC4 and Ligase IV do not have roles outside of NHEJ, whereas in contrast, KU acts in other processes such as transcription, apoptosis, and responses to microenvironment.

The NHEJ may be decreased or shut down in different ways, many of which directly affect the above referenced proteins (e.g., the heterodimer Ku80/Ku70, DNA-PKcs, but in particular DNA ligase IV, XRCC4, Artemis and XLF (XRCC4-like factor; or Cernunnos), PIKK (phosphoinositide 3-kinase-like family of protein kinases). However, while NHEJ is, in many application, undesirable the inaccurate repair of the double strand break general destroys the functionality of the gene in which the double strand break occurs, which is often all that is required in the context of the present invention. Accordingly, the present invention also includes embodiments, in which increase of the NHEJ is desirable.

### HR: Basic Mechanisms

Homologous recombination (HR) is a very accurate repair mechanism. A homologous chromatid serves as a template for the repair of the broken strand. HR takes place during the S and G2 phases of the cell cycle, when the sister chromatids are available. Classical HR is mainly characterized by three steps: 1) resection of the 5′ of the broken ends, 2) strand invasion and exchange with a homologous DNA duplex, and 3) resolution of recombination intermediates. Different pathways can complete DSB repair, depending on the ability to perform strand invasion, and include the synthesis-dependent strand-annealing (SDSA) pathway, the classical double-strand break repair (DSBR), the break-induced replication (BIR), and, alternatively, the single-strand annealing (SSA) pathway. All HR mechanisms are interconnected and share many enzymatic steps.

The first step of all HR reactions corresponds to the resection of the 5′-ended broken DNA strand by nucleases with the help of the MRN complex (MRE11, RAD50, NBN (previously NBS1, for Nijmegen breakage syndrome 1)) and CtIP (CtBP-interacting protein). The resulting generation of a 3′ single-stranded DSB is able to search for a homologous sequence. The invasion of the homologous duplex is performed by a nucleofilament composed of the 3′ss-DNA coated with the RAD51 recombinase protein. The requirement of the replication protein A (RPA), an heterotrimeric ssDNA-binding protein, involved in DNA metabolic processes linked to ssDNA in eukaryotes, is necessary for the assembly of the RAD51-filament. Then RAD51 interacts with RAD52, which has a ring-like structure to displace RPA molecules and facilitate RAD51 loading. Rad52 is important for recombination processes in yeast. However, in vertebrates, BRCA2 (breast cancer type 2 susceptibility protein) rather than RAD52 seems to play an important role in strand invasion and exchange. RAD51/RAD52 interaction is stabilized by the binding of RAD54. RAD54 plays also a role in the maturation of recombination intermediates after D-loop formation. In the other hand, BRCA1 (breast cancer 1) interacts with BARD1 (BRCA1 associated RING domain 1) and BACH1 (BTB and CNC homology 1) to perform ligase and helicase DSB repair activity, respectively. BRCA1 also interacts with CtIP in a CDK-dependent manner and undergoes ubiquitination in response to DNA damage. As a consequence, BRCA1, CtIP and the MRN complex play a role in the activation of HR-mediated repair of DNA in the S and G2 phases of the cell cycle.

The invasion of the nucleofilament results in the formation of a heteroduplex called displacement-loop (D-loop) and involves the displacement of one strand of the duplex by the invasive strand and the pairing with the other. Then, several HR pathways can complete the repair, using the homologous sequence as template to replace the sequence surrounding the DSB. Depending of the mechanism used, reciprocal exchanges (crossovers) between the homologous template and the broken DNA molecule may be or may not be associated to HR repair. Crossovers may have important genetic consequences, such as genome rearrangements or loss of heterozygosity.

The five Rad51 paralogs are also involved in homologous recombination: Xrcc2, Xrcc3, Rad51 B, Rad51C, Rad51 D. Rad51 paralogs form two types of complexes: one termed BCDX2 comprises Rad51B, Rad51C, Rad51D and Xrcc2; the other contains Rad51C and Xrcc3 (CX3). The first complex has been proposed to participate in the formation and/or stabilization of the Rad51-DNA complex. The role of the second complex seems to be branch migration and resolution of the Holliday junction.

As previously reported, increasing the HR relative to the NHEJ (see US patent pub. 20120231449, which is incorporated herein by reference in its entirety) can be used to enhance and/or facilitate transgene expression.

Advantages of decreasing or shutting down HR have also been described (WO 2014/118619, US Patent Publication 20150361451, which is incorporated herein by reference in its entirety. HR may be decreased or shut down in different ways, many of which directly affect the above referenced proteins (see also Table C; however it is noted that there is no clear differentiation between HDR different pathways). RNAs, such as siRNAs or shRNAs are generally used to accomplish the decrease or shutdown.

### Microhomology-Mediated End Joining (MMEJ)

When the other recombination pathways fail or are not active, DSBs can be repaired by another, error-prone repair mechanism, namely MMEJ. This pathway still needs to be fully characterized and is sometimes also referred to as alternative end-joining (alt-EJ), although it is unclear whether these two processes are based on the same mechanism. The most characteristic feature of this pathway, which distinguishes it from NHEJ, is the use of 5-25 bp microhomologies during the alignment of broken DNA strands and results, in contrast to the NHEJ pathway, to the deletion of larger stretches of nucleic acids in the target genome, e.g. more than 20, 40, 60, 80, 100, 150, 200 bps, which is advantageous in many embodiments of the present invention.

MMEJ can occur at any time of the cell cycle and is independent of core NHEJ and HR factors, i.e. Ku70, Ligase IV and Rad52 genes. Instead MMEJ initiation relies on its own set of proteins, the most important ones being the components of the MRN complex (MRX in yeast) comprising Mre11, Rad50 and Nbs1 (Xrs2 in yeast), also implicated in the first steps of HR (Ma et al., 2003). Apart from the MRN complex many other factors have been proposed to participate in MMEJ, and in the related DNA synthesis-dependent SD-MMEJ mechanism, e.g. CTBP-interacting protein, poly (ADP-ribose) polymerase 1 (PARP1), the ligase III/Xrcc1 complex, ligase I, DNA polymerase e (Yu and McVey, 2010), and the ERCC1/XPF complex. However, many more proteins may also take part in this process.

It has been suggested that in the absence of other DNA-end binding proteins (like Ku or Rad51) the DSBs are recognized by PARP1 which then initiates their repair through MMEJ. The repair process, similarly to HR, starts with 5′ to 3′ end resection, which exposes short regions of homology on each side of the break. This processing step is conducted by the MRN complex and regulated by CtIP. The complementary regions (present in the 3′ ssDNA fragments) pair together and the non-complementary segments (flaps) are removed, probably by the ERCC1/XPF complex. Gaps (if any) are then filled in by a polymerase (e.g. DNA polymerase θ or δ and breaks joined by the ligase I or ligase III/Xrcc1 complex.

In the absence of immediate microhomology regions at the DNA ends, which is most often the case, a more distant fragment of the repaired molecule can be copied using an accurate DNA polymerase (e.g. polymerase 0). This duplicated region then participates in the alignment of DNA ends, which results in an insertion in the created junction. This more complex variant of microhomology-mediated repair has been termed synthesis-dependent MMEJ (SD-MMEJ).

Although MMEJ was thought to act as an alternative recombination repair pathway, it has been shown to be very efficient in the process of IgH class switch recombination in B lymphocytes, suggesting that it might be more than a backup mechanism. It is also possible that some DSBs, e.g. incompatible overhangs or blunt ends (which are poor NHEJ and/or HR targets) might be more efficiently repaired by MMEJ.

FIG. 12B shows the increase of expression of rate limiting HR and/or MMEJ proteins stimulates gene correction in a dose dependent manner. As can be seen an empty vector provides a base level of 1, wherein certain doses of expression vectors providing the rate liming proteins shown increase, ergo upregulate gene correction, by more than 0.05 fold, more than 0.1 fold, 0.2 fold, 0.3 fold, 0.4 fold or 0.5 fold. However, as the person skilled in the art will appreciate any of the proteins shown, e.g., Table C can be upregulated accordingly.

### Detection of Possible Unexpected Adventitious Agents Such as Viruses

The characterization of all viral-like elements in the genome of a cell of interest such as a CHO genome allows for the characterization of potential new adventitious agents from master cell banked lines. This approach may replace many of the numerous assays that are currently being applied to characterize a producer cell clone, as needed for the regulatory approval of any biotherapeutic produced by cell lines. The characterization can also be used in the context of the analysis of production lots on a routine basis. Efficient CHO genome editing by targeted gene editing via, e.g. homologous recombination, may be used to remove expressed retroviral elements that are present in the genomes of cultured cells of interest, including CHO cells, such as CHO-K1 cells resulting in a reduced viral genomic load.

There are currently several available commercial services for gene editing or transgene-targeted integration that rely on homologous recombination mechanisms. These include the Zn-finger, Talen and CRISPR/Cas9 nucleases and Adenoviral vectors of e.g. SAFC-SANGAMO in the US, CELLECTIS in France, and HORIZON in the UK. However, two of these tools, Zn-finger and Talen, remain difficult to access and to use, as a specific nuclease and/or vector has to be engineered for each genomic sequence that is edited, whereas the availability of the third (CRISPR) remains highly uncertain. Furthermore, homologous recombination remains quite an inefficient process in the cell, requiring the screening of many candidate cell lines. Deleting the two alleles of a given gene thus often requires multiple rounds of mutagenesis and cell clone isolation and characterization. Thus, these current technologies are often too slow and too costly to allow for the systematic engineering of cellular genomes, as required for instance to remove multiple expressed viral gene remnants dispersed in the CHO genome. The engineering of metabolic pathways and recombination mechanisms in the CHO cell pave the way towards more efficient genome editing approaches by one of the recombination pathways, wherein, in most instances, homologous recombination is preferred.

To identify expressed ERV elements, the genome sequence of a CHO cell line (for instance SELEXIS'S CHO-K1-derived CHO-M cells) was determined with an approximately 120-fold coverage using the PACBIO (Pacific Bioscience Inc.) technology. The genome sequencing can be, e.g., performed in the PacBio RS II™ long-read sequencer.

The CHO genome was assembled in approximately 7200 contigs displaying an N50 size of 6.1 Mb.

### Identification and Characterization of Expressed ERV Elements in the CHO Genome

CHO genome sequences displaying sequence similarities to known murine retroviral sequences were identified using the BLAST algorithm, searching for the viral GAG, POL and ENV element coding sequences, with a sequence conservation of at least 80%, as well as the presence of the viral long terminal repeats (LTR).

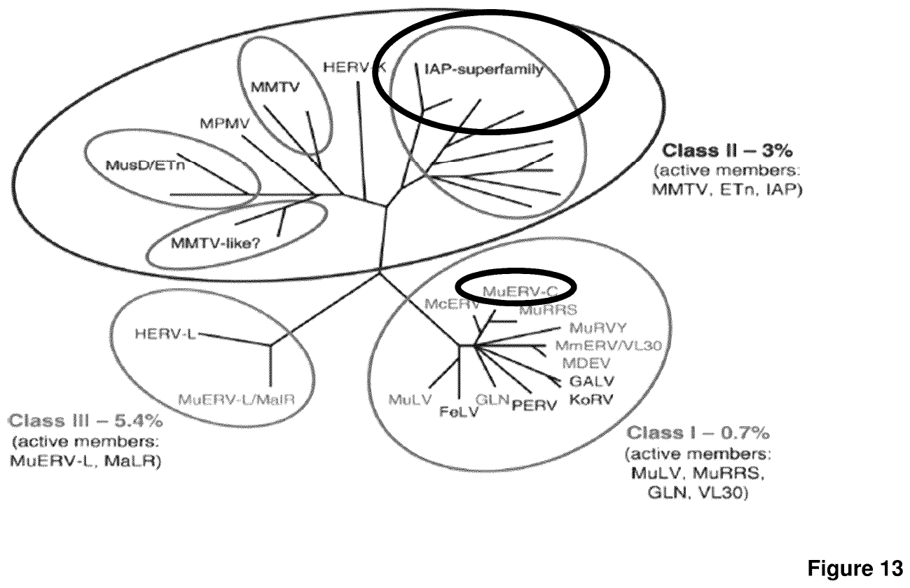

As depicted in FIG. 13, three classes of gammaretroviruses have been identified, all of which may contribute to ERVs or ERV elements found in cells of interest. ERV elements that are expressed to produce a functional protein are of particular interest in the present context. A database of viral, in particular retroviral and/or microbial-like sequences within cells of interest, serves as a starting point. A corresponding database of the respective RNA sequences of those cells provides information as to which of the DNA elements are actually expressed and may encode a functional protein.

### Construction of a Database of Relevant DNA Elements

Dispersed repeated sequences from past viral and retroviral integrations and retro-transposition events in the genome of cells of interest, e.g. cells used in the production of biotherapeutics, in particular those elements that remain expressed, are identified.

As a first step in identifying expressed ERV elements, the genome sequence of a CHO cell line (SELEXIS'S CHO-K1-derived CHO-M) was determined with a coverage of approximately 120-fold. For this, the PACBIO (Pacific Bioscience Inc.) technology was used. Such extensive sequencing can be, e.g., performed in the PacBio RS II™, original long-read sequencer. However, as the person skilled in the art will appreciate, other single molecule DNA sequencing technologies and apparatuses can equally well be used. Specific PCR amplifications and targeted DNA sequencing of relevant loci to obtain, e.g., a CHO-M genome sequence version 3.0 was performed.

The CHO-M genome was assembled in approximately 7200 contigs displaying an N50 size of 6.1 Mb.

To construct a database of DNA or cDNA indicator sequences for potential adventitious microbial contaminants, as based on list of adventitious agents whose screening is currently requested by agencies such as the FDA, genome sequences displaying sequence similarities to known murine retroviral sequences were identified.

To map their positions within the CHO-M genome scaffolds, CHO-M genome sequences displaying sequence similarities to known murine retroviral sequences (for instance the ML2G sequences, Lie et al, 2014) were identified using the BLAST algorithm, searching for the viral GAG, POL and ENV coding sequences, with a sequence conservation of at least 80%. The Blast alignment was run separately for each of these coding sequences. In addition, the presence of the viral long terminal repeats (LTR) was investigated, also via Blast with a threshold of 80%. The average sequence identify between the ML2G sequences and viral GAG, POL and ENV were determined to be 92%. The average sequence identify between GAG, POL and ENV and Mouse Leukemia Virus is 66%. In some instances, the ERVs were not complete, and only certain ERV elements were found.

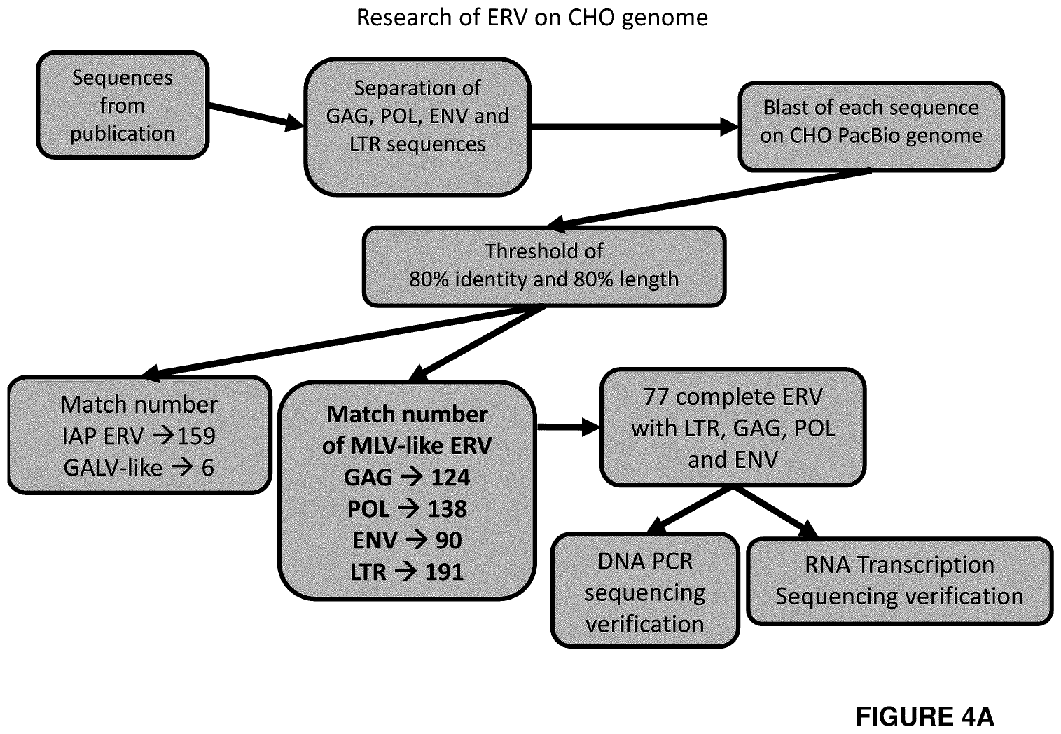

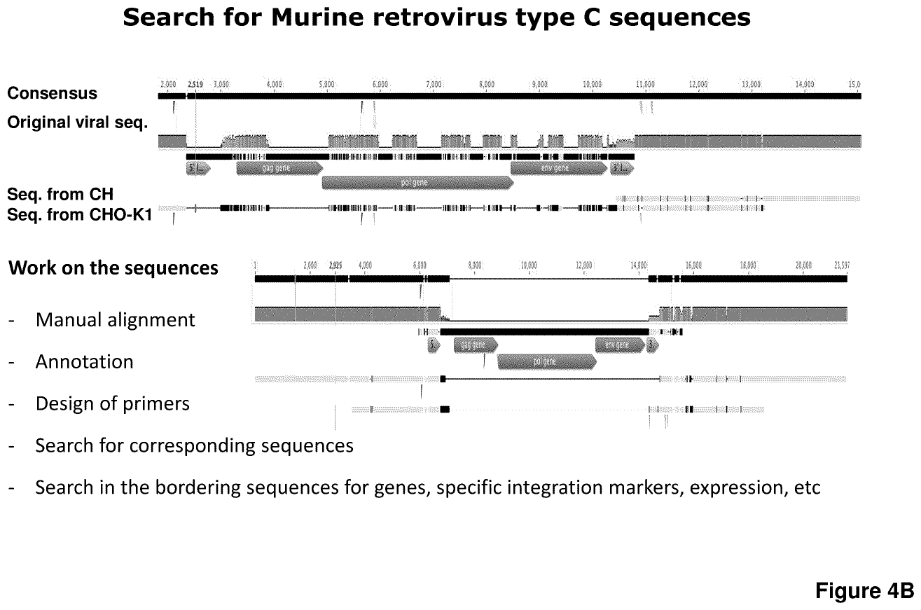

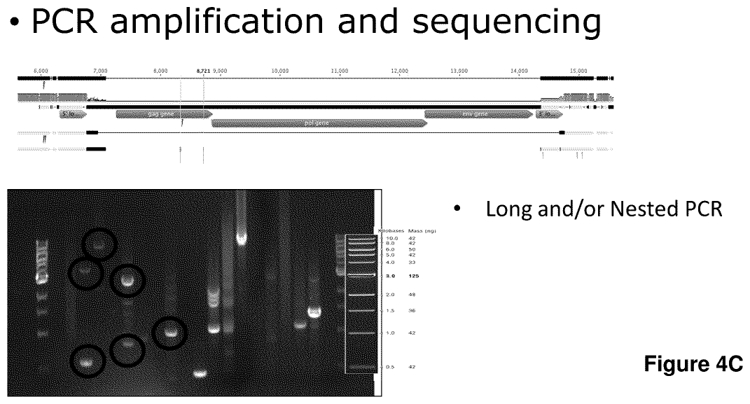

The identified sequences of ERVs included 159 members of the Intracisternal type A Particles (IAP) class II retroviruses, 144 Type C gammaretroviruses (class I ERVs), as well as 8 other gammaretroviruses, in particular GALV (see FIG. 13 for the main classes and phylogenetic relationship of these viral sequences). A typical search for a murine retrovirus type C sequence in a CHO cell is outlined in FIG. 4A.

The type C gammaretroviruses ERV sequences were further investigated and the number of ERV elements found are shown in Table A:

TABLE A

| Genes | GAG gene | Pol gene | Enveloppe | LTR |
|---|---|---|---|---|
| Extracted sequences | 124 | 138 | 98 | 191 |

After the sequences were found via alignment, primers were designed and, using these primers, the corresponding sequences were searched for experimental validation using PCR and DNA sequencing. Bordering sequences were searched for genes, specific integration marks, DNA methylation and expression, etc.

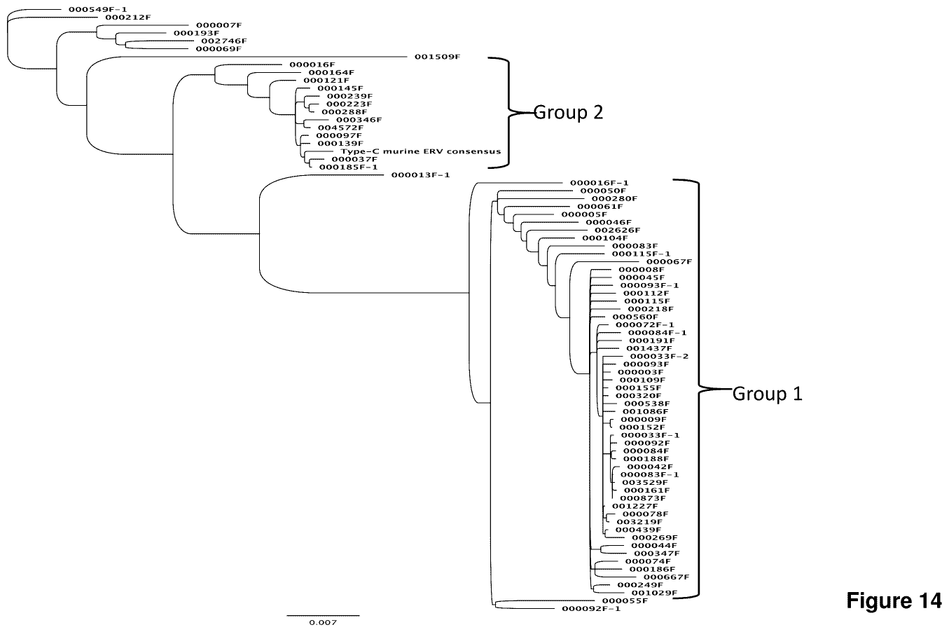

Only 77 of the ERV sequences analyzed had all the elements (gag, pol, env genes) to produce an ERV. Those were further analyzed and the results are shown in FIG. 14. These 77 ERV sequences were also phylogenetically analyzed and could be divided into two groups, namely into group 1 (52) and group 2 (14) ERVs. Within these groups there was 97-99% sequence identity and between the groups there was 84-97% sequence identity. The two groups differed in their LTRs. The GAG coding sequence was well conserved.

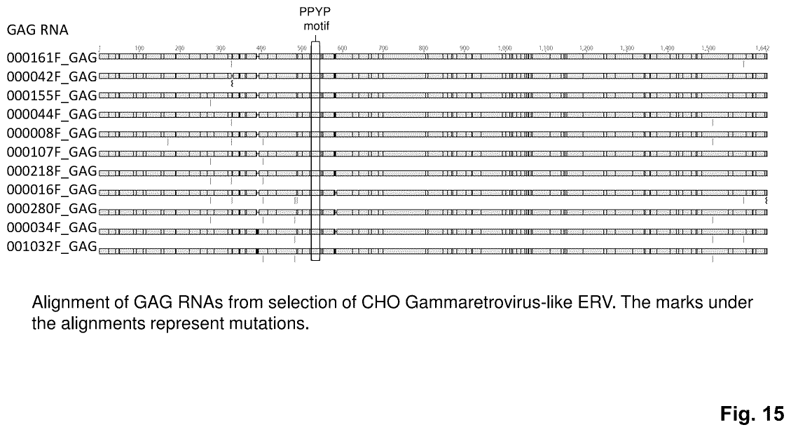

Sequence comparisons indicated that two burst of retroviral infections may have occurred relatively recently during the Chinese hamster evolution, giving rise to these well preserved and possibly functional ERVs (FIG. 15).

Furthermore, it was found that the DNA sequence encoding a PPXY-related PPYP motif important for GAG function was also present. The group 1 ERVs displayed one to twelve differences or 30 bp integrations in the genome, group 2 ERVs displayed none to 6 differences in the gag gene relative to related functional gag genes.

### Investigation of Potential Expression of Identified DNA Elements

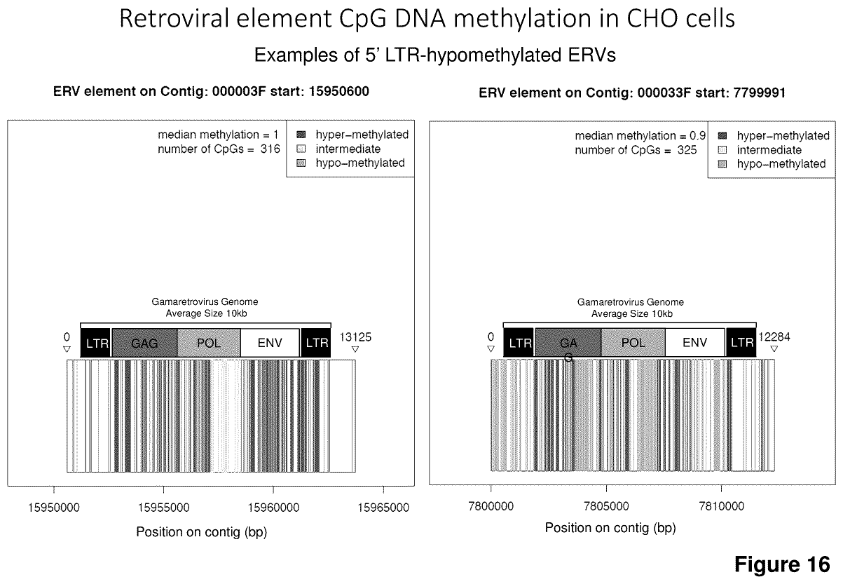

To assess which ERV element(s) may be transcribed, their CpG DNA methylation status was analyzed using the PACBIO sequences data (Suzuki et al., (2016)). This allowed the identification of several ERVs whose LTR sequences were hypomethylated, as expected for an expression permissive chromatin structure and transcriptionally active LTR promoter sequences, as exemplified in FIG. 16.

### Construction of a RNA Database

Further sequencing and analysis of several independent mRNA preparations of the banked cell, such as a CHO-M master cells may be carried out to construct a database of RNA (i.e. cDNA) sequences that map to the CHO M genome and/or to the indicator adventitious sequence database. The product is a collection of expressed retroviral/viral, retro-transposon and bacterial marker genes and families, providing a table of the relative expression levels for each type of element.

Abundant RNA sequences that cannot be attributed to the genomic or mitochondrial genomic DNA are validated experimentally by RT-qPCR. Experimentally validated sequences are searched for possible trans-splicing events. Remaining unexplained sequences are screened for homologies to available genomes of viral, prokaryotic and eukaryotic sequences, to possibly attribute them to identified microbial species. Relevant hits are added to the database of expressed sequences.

### Examples of a Specific Construction of a RNA Database of RNA Expressed by Identified DNA Elements

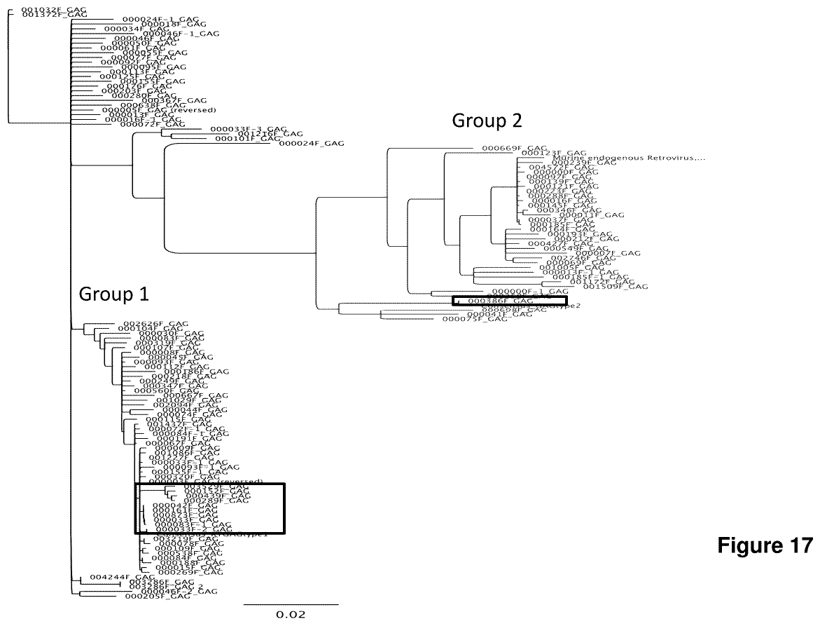

Here, the transcription of specific ERV elements was further assessed by their direct comparison with the sequence of GAG cDNAs generated using reverse transcription of the total CHO, here CHO-M cDNA, cell RNA and PCR amplification. This indicated that both group1 and 2 ERV elements were transcriptionally active. Comparisons of the genomic and cDNA sequences further indicated that one Group 2 ERV was transcribed, which however could not express a functional GAG protein due to STOP codons (FIG. 17). However, several of the identified Group 1 ERV sequences were expressed, their RNA sequence did not have premature STOP codons, and they thus appeared to mediate the expression of a functional GAG protein. Analysis of the adjacent POL coding sequences revealed that they were also functional, leading to the identification of several expressed and conserved ERVs as very strong candidates for the expression and release of retroviral particles from CHO cells. The complete consensus DNA sequence of these functional Group 1 ERVs is shown in SEQ ID NO. 1, whereas the consensus sequences of the group 2 ERVs, IAP elements and other gammaretroviral sequences identified here are provided in SEQ ID NOs, 2, 3 and 4.

### Characterization of Adventitious Elements From Whole Genome/Episome Sequencing:

A bioinformatics package is provided for quick identification of (i) novel mobile genetic and adventitious elements from whole genome and episome sequencing, and (ii) significant changes in the levels of expression of known microbial-like genes, as these may be indicative of the contamination of a cell culture by adventitious agents. The contaminations need to be distinguished from chromosomal rearrangements.

Here, genome comparison programs are used to devise automated processes to distinguish small genomic changes (such as transposition or viral genome integration) from large chromosomal rearrangements (such as large chromosomal translocation or duplication events) or from contamination by other cultured cells.

The parameters of the above discrimination process are determined using genomic and RNA sequences obtained from the banked cells such as CHO-M cells and from cell clones derived thereof, using clones that were previously validated for the absence of adventitious agents via conventional means.

The above process is used in conjunction with the databases obtained above, to identify potential genomic changes elicited during novel CHO cell clone isolation. Relevant hits are validated experimentally by PCR and by small scale DNA sequencing.

A similar analysis is performed using the transcriptome of, e.g., CHO-M cell clones. New RNA sequences, or RNA sequences whose expression level is significantly changed, are identified using bioinformatics analysis. The hits are validated experimentally by RT-qPCR.

The detection process is validated first in terms of sensitivity by bioinformatics modeling, followed by experimentally spiking-in CHO-M nucleic acid preparations with a known amount of a retroviral-like or bacterial (e.g. mycoplasma) sequences and with reference materials. This provides a sensitivity threshold in terms of the number of viral genome per cell genome equivalent that can be revealed following a given depth (or genome coverage) of “next-generation sequencing”.

### Process for More Efficient Editing of the CHO Cell Genome and/or Transcriptome

A quantitative assay for HR and HDR targeted transgene integration and removal from the CHO cell genome via the knock-down or overexpression of proteins involved in recombinant activities is provided.

Here, several independent indicator CHO cell lines are constructed that contain a single genome-integrated GFP transgene (e.g., via transposable vectors), and transgene integration is validated by quantitative PCR and integration site mapping. An indicator plasmid is constructed by bracketing a dsRed expression cassette by sequences corresponding to the extremities of the GFP coding sequence. The frequencies of spontaneous mutation of the GFP sequence and of non-targeted integration of the dsRed expression cassette is documented relative to proper targeted integration using fluorometric and qPCR assays. Alternatively, one or several deleted GFP coding sequence is/are integrated into the cell genome, and they are repaired using HR or HDR-related mechanisms, to restore a functional GFP coding sequence and fluorescent cells

The above assay is used following the transient siRNA-mediated knock-down of MMEJ and NHEJ proteins, so as to identify limiting activities that may oppose efficient HR mechanisms. A similar assay is performed in the presence of an expression vector for Cas9 and a GFP-targeted CRISPR guide RNA, to assess for HDR. In parallel, the cDNAs encoding CHO HR and HDR proteins are cloned and integrated into expression vectors. These cDNAs are co-transfected with the dsRed indicator plasmid to identify activities that limit HR or HDR efficiency in CHO-M cells.

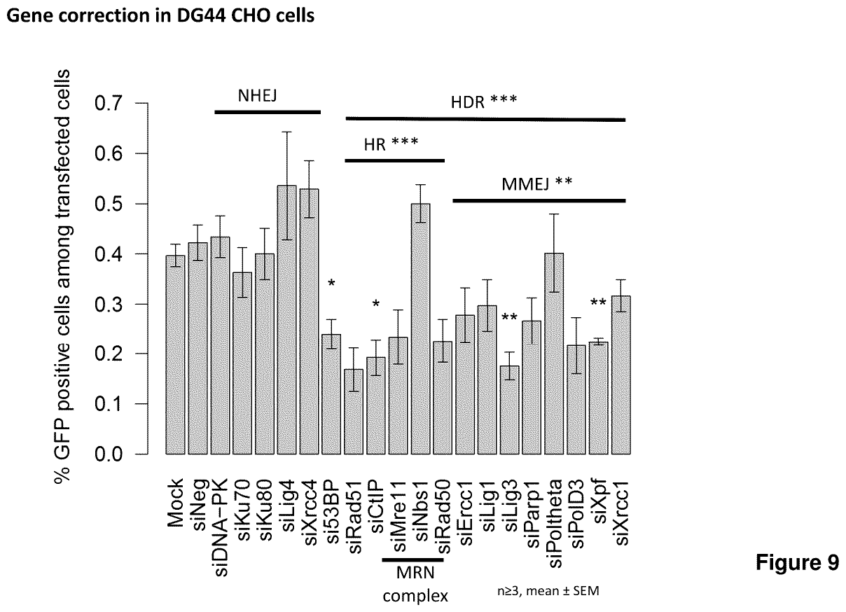

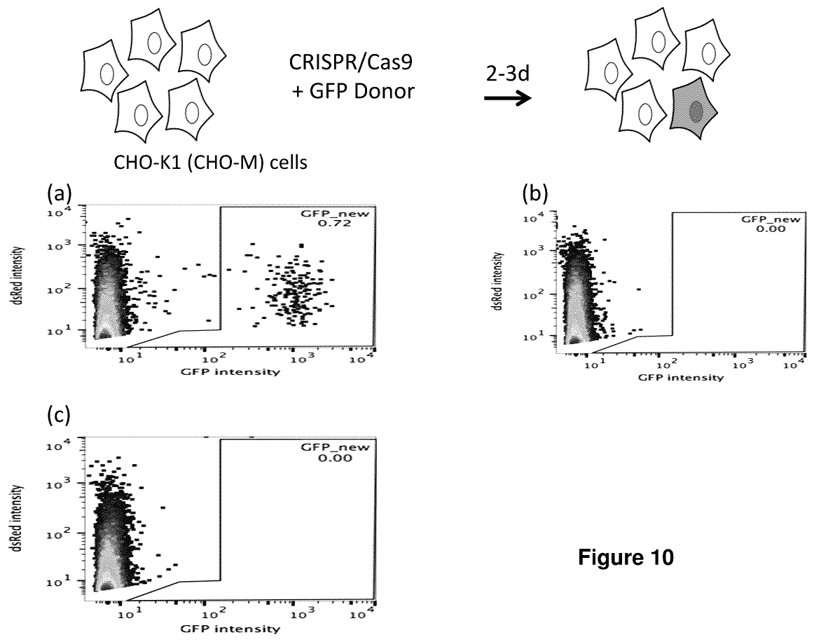

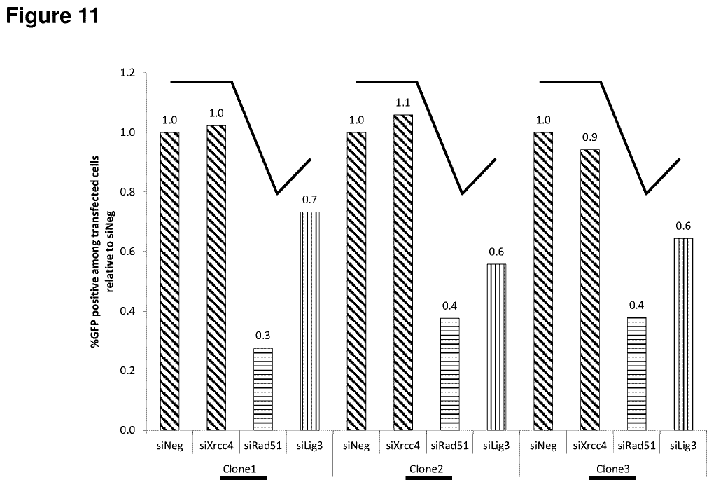

Combinations of the above knock-down or overexpression are performed to improve the frequency of HR-like events (see FIGS. 9 to 12). The most efficient combinations are assessed and selected based on i) the results of the above assays of single activities, ii) the mRNA levels of particular recombination genes in CHO-M cells, and iii) the relative positions in the recombination pathway and multimeric structures formed by the proteins. These combinations are assayed to remove the dsRed sequence integrated into GFP, to restore a functional GFP, in order to identify a process that efficiently deletes expressed sequences from the CHO genome.

### CHO-M Genetic or Epigenetic Editing to Remove Infectious Agent DNA or RNA Remnants

Here, expressed viral genome remnants from CHO cell lines are removed or silenced. Viral- and retroviral-like elements expressed by CHO-M cells as determined above are targeted by HR or HDR using the optimized approach above so as to disrupt functional viral sequences, preferably gag sequences that mediate viral particle release.

Alternatively, or concomitantly, the mechanisms mediating HR or HDR are inhibited, so as to promote the repair of DSB in viral sequences by repair mechanisms that promote deletions at the DNA cleavage site, such as NHEJ and MMEJ-related mechanisms, so as to delete gag sequences that are required for the release of viral particles.

Multiple rounds of genome editing are performed, and the reduction in genomic viral and of RNA sequences will be followed by quantitative PCR. The cell division timing will be characterized after each round to ensure that additional genomic changes do not impair the metabolic properties of the cells.

Suitably engineered cells such as CHO-M cell populations are tested for their stability and ability to express marker (e.g. GFP) or therapeutic (e.g. immunoglobulins) proteins. Subclones are generated and similarly assayed. Suitable subclones are documented for the lack of possible additional adventitious agents using the detection methods described herein.

The genome sequence of the most efficient cells such as CHO-M subclones is determined and assembled, to document the genomic changes as well as the reduction in adventitious viral-like sequences.

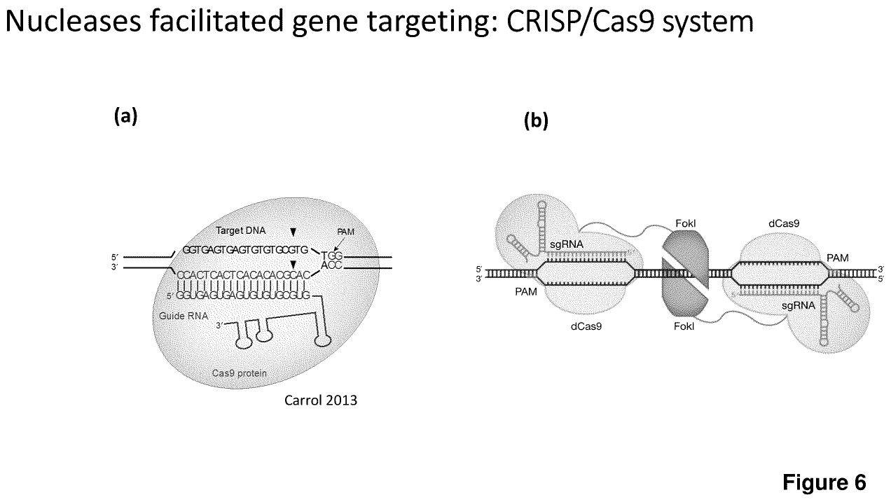

In order to specifically cleave and mutate the expressed Group 1 ERVs using CRISPR/Cas9 genome edition approach, guide RNAs were designed to recognize preferentially sequences around the PPYP motif or the myristoylation sequence of the GAG sequence (FIG. 18 and SEQ ID Nos 5 to 12). The PPYP motif was chosen for a knock-out approach whereby non-HR DNA repair mechanisms may lead to deletions in the GAG coding sequence that may impair the expression of a functional version of this protein which mediates viral particle budding. The myristoylation motif was targeted to substitute the myristoylated amino acid by a non-modifiable counterpart by homology-directed HR DNA repair, so as to generate a dominant-negative version of GAG that may be both non-functional and that may inhibit the function of possibly remaining unmutated GAG proteins, as based on the known requirement for HIV1 GAG protein myristoylation for HIV budding (Abdusetir Cerfoglio et al. (2014)). The single guide RNAs (sgRNA) were designed to fit the monomeric version of CRIPR/Cas9, as illustrated in FIG. 6 (Carrol 2013).

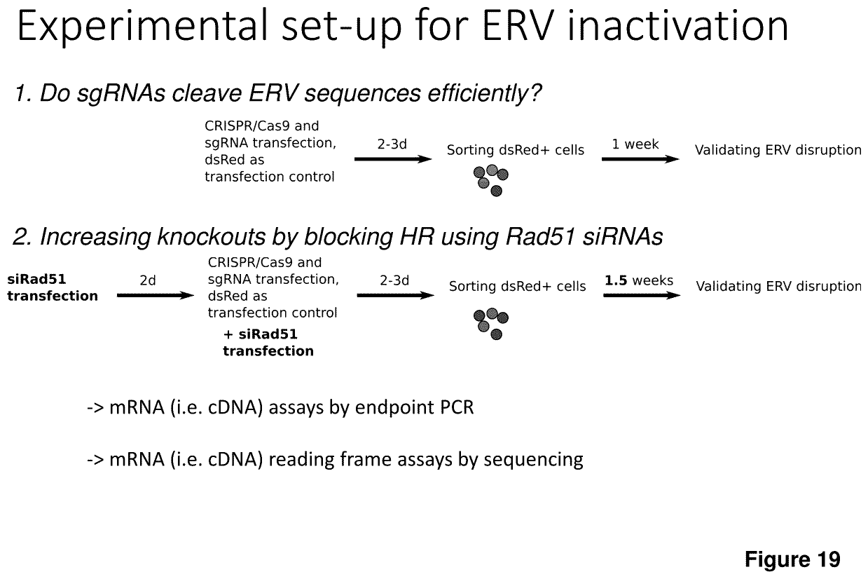

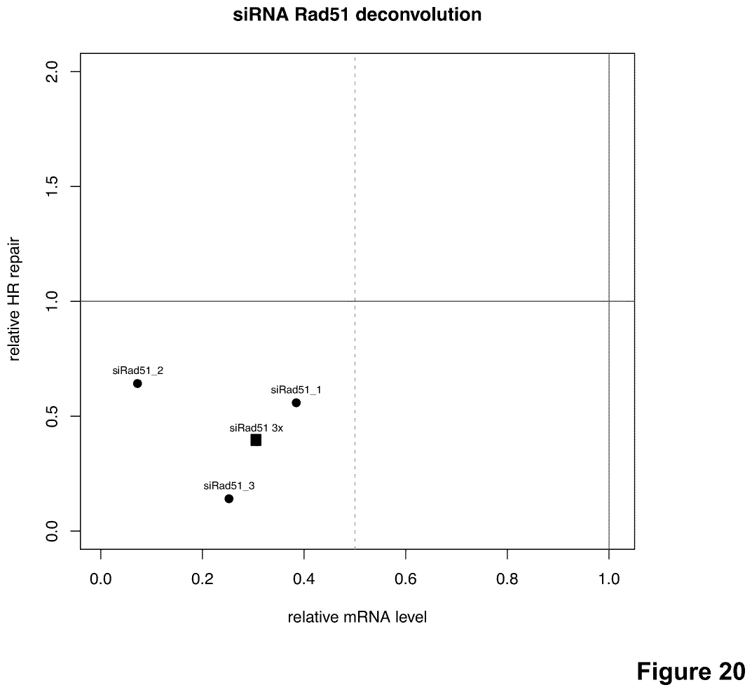

The Cas9 and sgRNAs expression vectors were co-transfected with a dsRed expression vector into CHO cells, and dsRed-expressing cells were sorted in order to enrich for the efficiently transfected and transgene-expressing cells (FIG. 19). Alternatively, the cells were transfected with a mix of three Rad51-targeting siRNAs, in order to decrease Rad51 expression and to reduce DNA repair by homologous recombination mechanisms. The efficiency of the Rad51 knock-down was assessed both at the Rad51 mRNA levels and by the inhibition of the restoration of the function of a mutated GFP transgene by homologous recombination repair, indicating that all designed siRNAs specifically inhibited Rad51 expression and HR (FIG. 20).

To directly assess whether the CRISPR/Cas9 treatment had properly targeted the expressed ERVs, the GAG RNA was reverse transcribed and PCR amplified. While GAG RNA was readily detectable from control cells, as expected, the cells treated with the PPYP5 sgRNA Cas9 consistently yielded low or undetectable levels in independent transfection experiments (1^{st} and 2^{nd} CRISPR experiments of FIG. 21). As treatments with the PPYP6 and PPYP13-programmed Cas9 did not significantly alter the GAG RNA levels when used alone, it was assessed whether the siRNA-mediated knock-down of Rad51 activity may increase the effect of these CRISPR/Cas9 treatment, as may be expected if this would prevent efficient repair of the cleaved gag genes by the HR mechanisms and thus may lead to deletion prone alternative DNA repair pathway. This was observed to be the case, as a prior knock-down of Rad51 yielded the disappearance of the GAG mRNA signal upon PPYP6 or PPYP13 and Cas9 treatment (3^{rd} CRISPR experiments of FIG. 21).

Previous attempts to mutate genes using the CRISPR/Cas or other nuclease-based mutagenic systems have not led to the disappearance of the encoded mRNA even for low copy genes. In the case of high copy number ERVs, which include ERVs present in the cell in copy numbers of more than 30, 40, or even 50, as found in mammalian genomes, it was expected to be even more difficult. Thus, it was quite surprising to observe that the retroviral RNAs were undetectable in cells treated with the PPYP5 sgRNA Cas9 nuclease (1s t CRISPR experiments of FIG. 21). A previous attempt to delete or mutate the POL gene of 62 related endogenous retroviral elements could not achieve their removal from pig cells using transient transfections, leading to the stable expression of CRISPR/CAS9 elements using transposable or viral vectors (Yang L. et al., 2015). Even so, the continuous activity of CRISPR/Cas for 17 days only led to a maximum targeting frequency of 37% of the cells, possibly because prolonged activity of CRISPR/CAS led to toxic off-target cleavage effects.

The experiments performed show that the knock-down of HR proteins such as Rad51 can be used to increase the frequency of GAG gene inactivation by deleterious deletions at a frequency not seen without transfecting the siRNA.

While the knock-down of Rad51 is provided as an example, someone skilled in the art will understand that the knock-down of other HR proteins will have comparable results.

Accordingly, CHO cells having other HR proteins knocked-down are within the scope of the present invention. But also cells having 53BP1, CtIP, Mre11, Rad50, Ligase III, Pold3 (DNA polymerase delta subunit 3), Xpf and Blm (Bloom syndrome RecQ like helicase) knocked-down (see FIG. 9) are specifically included in the present invention. Table C provides examples of HR proteins that may be knocked-down as well as proteins of other recombinant pathways that might be knocked-down or overexpressed. However, as the person skilled in the art will appreciate Table C is a simplification: Some of the proteins mentioned in Table C have dual function (e.g., Ligase III) and could be listed in different categories.

Without being limited to any particular theory, Cas9-cleaved ERVs may be repaired by homologous recombination taking one of the many other ERVs as a homologous template, which may be inhibited by the inactivation of homology-based pathway proteins such as Rad51. In contrast, some previous attempts to knock-down Rad51 expression prior to the expression of the CRISPR/Cas9 components has led to the increase of homology-directed DNA repair pathways, which must rather be avoided when deletions leading to gene inactivation are to be favored (Davis, L., and Maizels, N. 2014 and 2016). Other studies have found that Rad51 overexpression can increase homology-directed DNA repair in targeted gene integration (i.e. knock-in) studies following CRISPR/Cas9- or TALEN-mediated DNA cleavage (Song J et al (2016)). Here, it was shown that RAD51 favors HDR gene correction in various types of CHO cells (FIGS. 9 and 12), and that the inhibition of homologous recombination activities can greatly increase the mutation of ERV, when used in conjunction with an ERV-specific DNA nuclease. Thus, when multiple alleles are to be inactivated, ergo in case of high copy number ERVs, an embodiment is often preferred that combines siRNA knock-down, in particular of the HR pathway, in conjunction with, in particular CRISPR/Cas9 approach over the use of CRISPR/Cas9 alone, which is another embodiment of the present invention.

Overall, the very high mutagenic activity of CRISPR/Cas9 components is surprisingly elevated when compared to the state of the art. Without being limited to any particular theory, the reason may be that Cas9 nuclease specifically targets expressed ERV rather than inactive ones. However, this is unlikely to explain the reduction of the viral RNAs to undetectable levels in the most efficient settings. Thus, it may also be that specific DNA cleavage events can lead to the transcriptional silencing of expressed ERVs.

Finally, non-coding mutated RNAs bearing premature stop codons are often degraded intracellularly by the nonsense-mediated RNA decay (NMD) mechanism (Baker and Parker (2004). Thus, it was next assessed whether the PPYP sgRNA may mediate out of frame mutations that could lead to the NMD-mediated degradation of the GAG RNA. Such a frame shift mutation, generated by the deletion of 11 nucleotides from the PPYP motif of a group 1 ERV, is shown in FIG. 21 as an example.

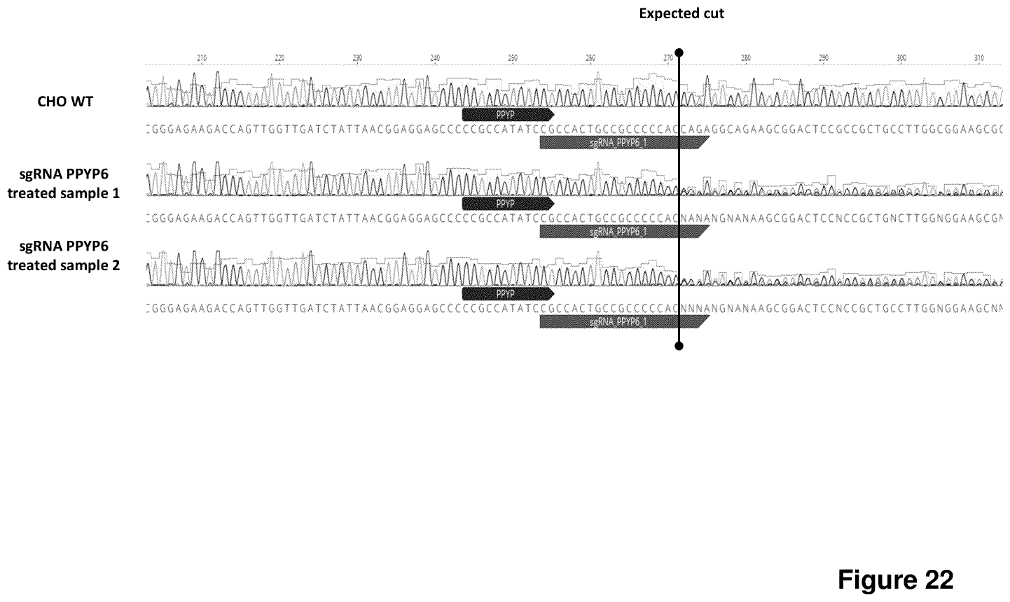

FIG. 22 shows the result of the PCR amplification and sequencing of the GAG genes contained in the polyclonal population of CRISPR/Cas-treated CHO cells. The results clearly indicate that a majority of the GAG coding sequences were mutated following a transient transfection of expression vectors for the CRISPR/Cas9 nuclease components, even when using the moderately active PPYP6 sgRNA and the absence of the siRAD51 treatment.

Overall, it was concluded that the proposed CRISPR/Cas9 approach targeting CHO cell endogenous ERVs can be used to eradicate or decrease the expression of the GAG protein that mediates viral particle release, and this without the need of prolonged CRISPR/Cas expression or the stable integration of their expression vectors into the cell genome, and of their potential negative effects upon cell culture. This approach can thus be used to generate safer CHO cells lines that are not able to express such retroviral proteins.

### Material and Methods

### Cell Line

The edited cell line was the SURE CHO-M cell line TM (SELEXIS SA, Switzerland), (see: U.S. Pat. Nos. 7,129,062 and 8,252,917, and published application numbers 20110061117, 20120231449 and 20130143264, the disclosures of which are incorporated herein by reference).

### CRISPR/Cas9 System

For genome editing we used the mammalian codon-optimized Streptococcus pyogenes Cas9 nuclease. Single guide RNA (sgRNA) sequences were cloned into the mammalian gRNA expression vector MLM3636 as previously described (Fu et al, 2013). To achieve high knockout efficiencies, several sgRNA target sites per target site were tested (PPYP: n=5; Myr: n=3). The CRISPRseek R package (Zhu et al., 2014) was used to identify ERV specific sgRNAs with minimal off-target sites using our private CHO-M genome as reference sequence. sgRNA sequences were designed to hybridize to the vicinity of the target sites, more precisely the Myr and PPYP motifs, to maximize the CRISPR/Cas9 mutagenesis potential for these loci. All possible sgRNA sequences were retrieved using the CRISPRseek R package (Zhu et al., 2014) that contain a 5′ 20nt NGG 3′ sequence structure and that mediate a DSB not more than 25 bp apart from the mutagenesis site. Among all potential sgRNA sequences, the sgRNA efficiencies were predicted using various scoring tools, including CRISPRseek (Zhu et al., 2014), Sequence Scan for CRISPR (SSC; Dana Faber Institute crispr website) and sgRNA scorer 1.0 (Dana Faber Institute crispr website). Furthermore, the CRISPRseek R package (Zhu et al., 2014) was used to identify ERV specific sgRNAs with minimal off-target sites using our private CHO-M genome as reference genome. Several sgRNA sequences per target site were finally selected and tested (Myr: n=3, PPYP: n=5), to identify sgRNAs with maximal mutation potential. Cas9 (#43861) and gRNA expression plasmids (#43860) are available from ADDGENE (Cambridge, MA, USA).

Table B Lists a Number of Example sgRNAs

| | Target sequence (5′-3′) with PAM | |
|---|---|---|
| | underlined; addition of non-targeting G | |
| sgRNA | to improve U6 expression (in lowercase) | Orientation |
| Myr2 (Seq ID No. 5) | gTCCTAAGCCTAGAAACTATGGGG | Forward strand |
| Myr4 (Seq ID No. 6) | gCATAGTTTCTAGGCTTAGGAGGG | Reverse strand |
| Myr8 (Seq ID No. 7) | GAGTGTTAGGGACAAAGGAGTGG | Reverse strand |
| PPYP5 (Seq ID No. 8) | GTTGGTTGATCTATTAACGGAGG | Forward strand |
| PPYP6 (Seq ID No. 9) | GCCACTGCCGCCCCCACCAGAGG | Forward strand |
| PPYP7 (Seq ID No. 10) | GCCCCCACCAGAGGCAGAAGCGG | Forward strand |
| PPYP13 (Seq ID No. 11) | GGCAGTGGCGGATATGGCGGGGG | Reverse strand |
| PPYP20 (Seq ID No. 12) | GCTTCTGCCTCTGGTGGGGGCGG | Reverse strand |

### RNA Interference

Small interfering RNA (siRNA) against the Rad51 CHO homologous were designed and synthesized by MICROSYNTH AG (Balgach, Switzerland). Rad51 mRNA levels were silenced using a mixture of three specific Rad51 siRNA to minimize off-target effects. BLAST analysis excluded homology to other targets. A mixture of three non-targeting siRNAs (siNeg) was used as control.

### Modifications of CHO-M Genome

CHO-M cells were transfected using the Neon Transfection System® (INVITROGEN) according to the manufacture's protocol. Briefly, 400,000 cells were transfected with 100 nM siRNA mixture and 48 h later, 700,000 cells were re-transfected with the CRISPR/Cas9 system using a dsRed encoding plasmid as transfection control. To enrich for transfected cells, around 100,000 dsRed positive cells were polyclonaly sorted 48-72 h after transfection using flow cytometry.

As the person skilled in the art will appreciate, the above description is not limiting, but provides examples of certain embodiments of the present invention. With the guidance provided above, the person skilled in the art is able to devise a wide variety of alternatives not specifically set forth herein.

TABLE C lists some of the key genes in each of the three pathways, (see also US Patent Publication 20120231449, which is incorporated herein by reference in its entirety). Also included in the table are DNA repair proteins such as MDC1 and MHS2. MDC1 is required to activate the intra-S phase and G2/M phase cell cycle checkpoints in response to DNA damage. However, MDC1 also functions in Rad51-mediated homologous recombination by retaining Rad51 in chromatin. (For sequences see, WO 2014/118619, US Patent Publication 20150361451, which is incorporated herein by reference in its entirety)

### Selected Genes of Certain Recombination Pathways

| Recombination | | |
|---|---|---|
| pathway | gene | Full name of gene |
| NHEJ | Xrcc4 | X-ray repair complementing defective repair in Chinese |
| | | hamster cells 4 |
| | Ku70 | X-Ray Repair Complementing Defective Repair In Chinese |
| | | Hamster Cells 6 |
| | Ku80 | X-ray repair complementing defective repair in Chinese |
| | | hamster cells 5 (double-strand-break rejoining) |
| | LigIV | Ligase IV, DNA, ATP-Dependent |
| | DNA-PKcs | Protein Kinase, DNA-Activated, Catalytic Polypeptide |
| | 53BP1 | Tumor suppressor p53-binding protein 1 |
| HR | Rad51 | RAD51 recombinase // DNA repair protein RAD51 |
| | Rad51B | RAD51 paralog B // DNA repair protein RAD51 homolog 2 |
| | Rad51C | RAD51 paralog C // DNA repair protein RAD51 homolog 3 |
| | Rad51D | RAD51 paralog D // DNA repair protein RAD51 homolog 4 |
| | Rad52 | RAD52 // DNA repair protein RAD52 |
| | Rad54 | RAD54 // DNA repair and recombination protein RAD54 |
| | Xrcc2 | X-ray repair complementing defective repair in Chinese |
| | | hamster cells 2 // DNA repair protein XRCC2 |
| | Xrcc3 | X-ray repair complementing defective repair in Chinese |
| | | hamster cells 3 // DNA repair protein XRCC3 |
| | Brca1 | breast cancer 1, early onset // breast cancer type 1 |
| | | susceptibility protein |
| | Brca2 | breast cancer 2, early onset // breast cancer type 2 |
| | | susceptibility protein |
| | Cyclin D1 | Cyclin D1 |
| | Bard1 | BRCA1 associated RING domain 1 // BRCA1 associated |
| | | RING domain 1 |
| MRN | Mre11 | Mre11 = meiotic recombination 11 // Double-strand |
| | | break repair protein MRE11 |
| | Rad50 | RAD50 Homolog (S. Cerevisiae) |
| | Nbs1 | Nibrin |
| MMEJ | Ercc1 | excision repair cross-complementing rodent repair |
| | | deficiency, complementation group 1 // DNA excision |
| | | repair protein ERCC-1 |
| | Xpf | excision repair cross-complementing rodent repair |
| | | deficiency, complementation group 4 |
| | Pol theta | Polymerase (DNA directed), theta |
| | Ligase I | DNA ligase 1 // DNA ligase 1 |
| | Ligase III | Ligase III, DNA, ATP-Dependent |
| | Xrcc1 | X-ray repair complementing defective repair in Chinese |
| | | hamster cells 1 |
| | CtlP | mediator of DNA-damage checkpoint 1 // Mediator of |
| | | DNA damage checkpoint protein 1 |
| | PARP1 | mediator of DNA-damage checkpoint 1 // Mediator of |
| | | DNA damage checkpoint protein 1 |
| | POLD3 | |
| DNA repair | MDC1 | mediator of DNA-damage checkpoint 1 // Mediator of |
| proteins | | DNA damage checkpoint protein 1 |
| | MSH2 | mutS homolog 2 |

### Bibliography

Abdusetir Cerfoglio et al., Structural elements in the Gag polyprotein of feline immunodeficiency virus involved in Gag self-association and assembly, Journal of General Virology 95,2050-2059 (2014).
Baker, K. E.; Parker, R., Nonsense-mediated mRNA decay: Terminating erroneous gene expression”. Current Opinion in Cell Biology 16: 293-299 (2004).
Carrol et al., Staying on target with CRISPR-Cas, Nature Biotechnology 31, 807-809 (2013).
Davis, L., and Maizels N., Homology-directed repair of DNA nicks via pathways distinct from canonical double-strand break repair. Proc. Natl. Acad. Sci. USA 111, E924-E932 (2014).
Davis, L., and Maizels, N., Two Distinct Pathways Support Gene Correction by Single-Stranded Donors at DNA Nicks. Cell Reports 17,1872-1881 (2016).
Food and Drug Administration (FDA), FDA's Guidance for Industry Characterization and Qualification of Cell Substrates and Other Biological Materials Used in the Production of Viral Vaccines for Infectious Disease Indications, February 2010, available at the Fda.gov website.
Fu et al., High-frequency off-target mutagenesis induced by CRISPR-Cas nucleases in human cells. Nat. Biotechnol. 31,822-6 (2013).
Guilinger et al., Fusion of catalytically inactive Cas9 to Fokl nuclease improves the specificity of genome modification, Nature Biotechnology 32,577-582 (2014)
Hope & Trono, HIV, Structure, Expression, and Regulation of the HIV Genome, Site Knowledge Base Chapter November 2000.
International Patent Publication WO 2014/118619 (2014).
Kostyrko et al., MAR-Mediated transgene integration into permissive chromatin and increased expression by recombination pathway engineering, Biotechnology & Bioengineering, 3 Oct. 2016 online edition.
Lewis et al., Genomic landscapes of Chinese hamster ovary cell lines as revealed by the Cricetulus griseus draft genome, Nature Biotechnology 31,759-765 (2013), Supplement)
Lie, Y. S. et al., Chinese hamster ovary cells contain transcriptionally active full-length type C proviruses. J. Virol. 68:7840-7849 (1994).
Ma et al., Hairpin opening and overhang processing by an Artemis:DNA-PKcs complex in V(D)J recombination and in nonhomologous end joining. Cell 108, 781-794 (2002).
Song J et al., RS-1 enhances CRISPR/Cas9- and TALEN-mediated knock-in efficiency, Nature Communications 7:10548 (2016).
Suzuki et al., Ag In: measuring the landscape of CpG methylation of individual repetitive elements. Bioinformatics 32.2911-2919 (2016).
US Patent Publication 20120231449 (2012).
US Patent Publication 20150361451 (2015).
Yang L. et al., Genome-wide inactivation of porcine endogenous retroviruses (PERVs), Science 350:1101-1104 (2015).
ADDIN Mendeley Bibliography CSL_BIBLIOGRAPHY Zhu et al., CRISPRseek: A Bioconductor package to identify target-specific guide RNAs for CRISPR-Cas9 genome-editing systems. PLoS One. 9 (2014).

1.-18. (canceled)

19. A method for improved genome editing comprising:
(i) introducing into a cell having a genome a heterologous system for introducing single or double stranded brakes into a target nucleic acid sequence of the genome, wherein the target nucleic acid sequence of an comprises at least one ERV (endogenous retrovirus) element comprising:
SEQ ID No: 30, or
a sequence having more than 90% sequence identity with SEQ ID No: 30, wherein the SEQ ID No: 30 or the sequence having more than 90% sequence identity with SEQ ID No: 30 is altered to comprise
at least one deletion, at least one addition, at least one substitution or combinations thereof resulting in an altered sequence, wherein the at least one ERV element comprising the altered sequence does not encode a functional Gag protein, and further
(ii) introducing into the cell,
(a) heterologous nucleic acid sequences encoding or activating one or more proteins of one or more homologous recombination (HR) pathways, and/or
(b) heterologous nucleic acid sequences encoding one or more sequences or proteins suppressing expression of the one or more proteins of one or more of the HR.

20. The method of claim 19, wherein the heterologous nucleic acid sequences of (ii)(a) are introduced into the cell.

21. The method of claim 19, wherein a marker gene is inserted into said target nucleic acid sequence via homologous recombination and wherein cells comprising the marker gene are selected.

22. The method of claim 19, wherein the heterologous nucleic acid sequences of (a) encode or activate proteins of the MNR complex.

23. The method of claim 19, wherein the deletion or the insertion is introduced into the at least one ERV element via non-homologous end-joining (NHEJ) or microhomology mediated end joining (MMEJ).

24. The method of claim 19, wherein the heterologous sequences are part of integrating vectors.

25. The method of claim 19, wherein the system for introducing single or double stranded brakes into the target nucleic acid sequence is a CRISPR (Clustered Regularly Interspaced Short Palindromic Repeats)/Cas9 (CRISPR associated protein 9) system or is based thereon.

26. The method of claim 19, wherein the cell is a CHO (Chinese Hamster Ovary) cell.

27. The method of claim 19, wherein the cell is a CHO cell comprising more than 10, 20, 30, 40, 50, 60, 80, 90 or 100 ERV elements each comprising the altered sequence.

28. (canceled)

29. A kit comprising:
(i) in one container at least one non-integrating vector encoding at least a nuclease,
(ii) in the container of (i) or a further container one or more guide RNAs or sequences encoding the one or more guide RNAs targeting a motif in a ERV element
(iii) in the container of (i) or a further container one or more siRNAs or sequences encoding one or more siRNAs, and
in a further container instruction of how to provide (i), (ii) and (iii) within a cell having a genome, wherein the genome comprises the ERV element, the ERV element comprising:
SEQ ID No: 30, or
a sequence having more than 90% sequence identity with SEQ ID No: 30,
wherein the SEQ ID No: 30 or the sequence having more than 90% sequence identity with SEQ ID No: 30 is altered to comprise
at least one deletion, at least one addition, at least one substitution or combinations thereof resulting in an altered sequence, wherein the at least one ERV element comprising the altered sequence does not encode a functional Gag protein.

30. The kit of claim 29, wherein the sequences encoding the one or more guide RNAs targeting the motif in the ERV element and/or the sequences encoding one or more siRNAs are part of a vector.

31. The kit of claim 30, wherein the vector is the non-integrating vector of (i).

32. The kit of claim 29, wherein the ERV element encodes a Gag protein.

33. The kit of claim 30, wherein the motif is a PPXY motif.

34. The kit of claim 29, wherein the siRNA is directed against a gene of the HR pathway.

35. The method of claim 29, wherein the nuclease is CRISPR-Cas nuclease.

36. The method of claim 19, wherein the heterologous nucleic acid sequences encoding the one or more sequences suppressing expression of the one or more proteins of the one or more of the recombination pathways are siRNAs and are introduced into the cell.

37. The method of claim 19, wherein the heterologous sequences of (i) and/or sequences of (ii) (a) and/or (ii) (b) are transiently expressed in the cell.

38. The method of claim 19, wherein the heterologous sequences are part of non-integrating vectors.

39. The method of claim 19, wherein the heterologous sequences encode Rad51.

40. The method of claim 21, wherein the marker gene is GFP.

41. The method of claim 22, wherein the proteins of the MNR complex encoded or activated are Nbs1 and/or Mre 11.

42. The method of claim 19, wherein each of the ERV elements comprises a deletion comprising more than 10 nucleic acids.

43. An engineered cell, preferably of a mammalian cell line such as an engineered CHO cell produced by a method such as the method of claim 19, including an engineered CHO-K1, comprising:
a genome of the cell, wherein the genome comprises one or more alterations comprising:
deletions,
additions, and/or
substitutions,
of one or more nucleic acids in one or more, generally, more than 10, 20, 30, 40, 50, 60, 80, 90 or 100 endogenous retrovirus (ERV) elements which are part of said genome, wherein the one or more alternations are preferably deletions.


SEQUENCE LISTING (32 sequences)

SEQ ID NO: 1 - DNA, 8366 bp, Cricetulus griseus
  misc_feature 1..8366: note = Group 1 type ERV of CHO-K1 cells
  source 1..8366: mol_type = genomic DNA
tgtaccagacaccagaccttgagaatatgctgatctggaatggctctgtgtctcatttgaaccatccaatggaaatgattctgtatttcgcctcatttgaaagactctgtgtttcacctcatttgaataactctgtactttacctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggagggactgacgggtccgaactcccgaccgcggctccaggagacgtcctggtagcgtttgaagccctcaggaagagggatttgtattttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatgggcaaactgtcaccactcctttgtccctaacactctcccactggaaagatgtacaggaatatgctcataaccaatctgttaatgtgcgtaaacgcaaatggattactctttgttcttcagaatggccgacctttgatgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttccccctctaaccgctccaaccgacccattccttcggccctacacctcccactcctttgattcctcccaacccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcggaagcggccctgacccttcaccaatggcttatcgactaagaggtcgtagggagcagcccgttccagattcaaccactctgcccctccgaactgggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggaagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccgggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggagaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccggccaagcttccaccgccaagggtttctgacctcctgaacctagaagattagaggagtcggggccaggagcccccccctgagccaggataacactgcaagtcggggggcatccggtcaccttcctagtcgatacaggggcacaacattccgttctgaatcggtcacccggacccctgagtcacaggactgcatgggtacagggagctacaggcggaaagcagtaccattggactacaaatcggcagctccagctcgcgaccggtaaggttatgcattctttcctccatgtgccagactgcccctaccccttactagacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtacaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccggttaggtaccccgctctaatcctcttacaatatgtagatgacattctcctggcagccaaaaccaaagggaaatgcaaggaaggcactcaagccctcctccagactcttgggagcctaggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaatctggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagatagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactccccctggaacttccgcacagagggcagaactcatagcactcacgcaggctctaaaattggcagaaggtaagaggctcaccgtgtatacagacagtcgttatgcctttgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaagggattctttcgaagcaagggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttgggaaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttaaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttattggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggcctccaactagtacaacgggaggtctggaaaccccttgctcaagcttataaagaccagagggaccatcccaccatcccccattcctaccagatcgggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccaacccaccgattcagccactgcatcagaatggaccgcacaccgcactcaaaatcctttaaagataagactctctcgtacaccctcctgttgattggttgtctgtttaccccccatgtagcaactaacccccacagggtttataatatcacctggaaaatagccaatctagggaccggggaaatagccaacctcagcacttatatagggactctacatgatgggttccctcctctctatgtcgacctatatgacttagtagggtctgattgggatccctctgaccaggaaccattcccagggtacggatgccaccaccctggggaaggataggaacaagaagcaaggatttttatgtttgccccggccataaaccaactcatggctgcgggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagttgggccaaacatagtaatagcggaccagagatcccaacccattttcaagtccctaaaccccctaccgttcctaaagctatcactcctacaccaggtgctgtcaccttctcccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccactaaagcaaatagatccagagtatctagagacctctgaatgaaagattccattcagttacaagagaaatgggggaatgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgccccaaggtcagccatcaggaaaaggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcgggcgcttagccacacagaagctaagtcgcccaggtacctgcgtctccaataaagcctcttgtttttacatccagttcgtggcctcgctgattcctgggtgtgtgggtctccctctacgaaagtgcctcttcggggtctttca

SEQ ID NO: 2 - DNA, 8667 bp, Cricetulus griseus
  misc_feature 1..8667: note = Group 2 type ERV of CHO-K1 cells
  source 1..8667: mol_type = genomic DNA
ctctctctctctctcttgggctagctaaattacagaccaagagaccaagaccacccagaaccatttctgagaatagaaggtcagggcgttatgactacttgtttgccacacaggatatctgtggtcagccatctggacgcggagggaggaactgccgggtctgttgtaaccactgtgtacccaagatagagccaagtcagcttcgtctgattaccagaccttgagaacttgctaacctgaaataactctgtgcctcatttgaattgtccaatagagtccctgtaactatgcttttcgcttctgtactatgctttttgctatataaggacccctctcccttggctcggcgtgcttggcctcacagaagctaagtcgccccaggtacccgtgtatccaataaagcctcttgctgtttgcatccaagttcatggtctcgctgattcctgggtacgggtctccttctgtgaaagtacctcttctgggggtctttcatttgggggctcgtccgggatcgaaaccccccgcccagggatcaccgacccacgtctgggaggtaagcgttgtgcggatccgctgtcttgtctgtcttgtcttgcctgtctgtctgaatctgtcttatgaactgcgcttgcgtctgtagtacacagctgcgtacatctgtatctggcggctccgaggaaaaactgaagagttcagactcccgaccacggctccaggagacgtcctggtagcgtctagagcctcagtgggctcactcttttttctgactagggtagatgcctaggcatcttcccaatctgtttggggaagcccctcagattgagggatttgctatcctcatcggggacgaggaagggaggcccccttcttcggccccatctgaagttttactttcgaccttgtgtcgaaaccgcgccgcaaaagcctgttctgtgttgctcggtctttgtcttgtaaccatcatttgtgtcctcctaggtctagaaactatggggcaaactgtcaccactcctttgtccctaacactctcgcactggagagatgtgcaggattatgctcataaccagtctgttgatgtgcataaacgcaaatggattactctttgttcctcagaatggccaacctttgacgtgggctggccgcgagatggtacctttaacccccaaactatatttcaggtgaaagagaagactatggatcctggaccacaggggcatcccgaccaggtggattacatagtcacttgggaggccttggttcaggaccctccccctgggtacgtcctttcttacaccccaagggcccctccttcctttccccctctaactgctccagccgacccactccctcggcccctacccttcccaatccctcgacacccactcctccctaccctcctccccattccaacctttaccctacagcggtaaaagacgctaaagctgaagaaaagaagacacctgaggtactcccccgggagaagaactgttggttgacttactgactgaggaacccccgccatatccagcaccgccaccgcccccaccagaggcagaagcggcccccgccgcctcggtggaggtggccccagctcccccacctgacccttccccaatggctcaccgactgagaggtcgcagggagcagcccgctccagattcaaccactctgcccctcagaactggaccgaacggccattctcagcctcggacctctataactggaaaaataacaatcctcctttctctgcagaccctgtgaggctgacatctctcatagaatcggtcctcacaactcaccaacctacctgggatgattgtgaacagattttgcaggttctcttaacctcggaggagaaacagcgcgttctactagaggcacgaaagaatgtcccaggagccaacgggcagcccacccagctgcccaatgaaatcgatgcggcttgccctcttgaaagacctgaatgggattttaccactgaagcaggtaggacccatctacgtctctatcgccagttgctcgtagcgggtctccggggggcaggacacccacccaccaatttggcccaggtaaagcaggtaatacagggggcggaggaatcgcccgccacttttctagagagactaaaagaagcgtataggatgtatactccctatgatccggaagatccagggcaggccaccagcgtttctatgtcctttatttggcagtcagccccggacataagaaacaagcttcaaaggctagaaaatttgcagggatatacactccaagatttgttgaaggaagcagaacgtatttttaataagagagagacacagacagaaagagaagaacgctggagaaaggaaacccaggagagagaggaaagactgagaaaagaagctgaggaaaaagaggctgcaagagaccgtaagcggaatagagagatgagcaggctattggccacagtagtgacaggtcagagacagaataggcagagggatggcagaagggggccccacctggacagggatcaatgtgcttactgcaaggaaaaaggacattgggcaagagattgccctaagaaccccgggccaagcctccacggccaagaacctctgaccttctaaacctagaagattagagaagtcagggccaggagcccccccccctgagcccaggataacactgcaagtcggggggcatccggtcaccttcctagtcgatacaggggcacaacattccgttctgaatcagtcacccggacccctgagtcaccggactgcatgggtacagggggctacaggcggaaagcagtaccgttggaccacagatcggcagctccagctcgcgaccggtaaggtcatgcactctttcctccatgtgcctgattgcccctaccctttactaggacgggacctattgaccaaactaaaagctcaaatacactttgagaaatcaaaagtcaaagtcacagggccagaaggaattccccttaccatcttgacaatgtccatagaagatgaatatagactccatgaaaaagagactaactcgggcaaccaggaaacccttgaccactggctcgcggaatttccccaaacctgggctgagacaggcggaatgggccttgccagcaaccaggccccaattatagtaaccttaaaagctgccgcccttcctgcatccgtcaagcagtacccgatgcctaaagaggcccgcgaaggaaacatattaaaaggttacttgaacaagagattctgacgccctgtaaatctccttggaatacacccttgttgcccgttaggaagccaggaaccaatgactatagaccagtgcaggatctgagagaagtcaataaaaggatagaggacatacaccctactgtccctaacccttacaatttgctgagcggattgccacctgactatacctggtatacagtcttagatcttaaagatgccttcttctgcctccacctgcatcccaccagccagcctatatttgcctttgaatggcaggacgctgaccttggaatctctgggcagctaacttggactaggttacctcaagggtttaagaacagccctaccctttttgatgaagctttacatcaggacctggcaggattccaggttcggtacccggctctaatcctcttacaatatgtagatgacatcctcctggcagccaaaacccaggggaatgtaaggagggcactcaagccctcctccagactcttgggagcctagggtaccgagcatccgccaagaagtcccagatatgtcagaagcaggtgacctatttaggatacaagataaaggacggactgcgatggctaacggaagcccgtatgtgagccatcttagaccttcccaccccgcaaaatccccaccaactaagagagttcttgggaacggcaggtttctgccgcctatggatccccgggtttgctgaaatggcggctcccctctaccccctcactcgtccagggattgcttttaaatgggaagagccccaaaagaaagccttcaccaacatcaaaaaggctctccttgaatcaccagccctgggactaccggacttggctaagccatttgagttctttgtagatgagaaagagggctatgccaagggagtcctcacccaaaaactggggccttggagaaggcccactgcatacctctccaagaaattggatcctgtggcatcgggatggccaccctgcctccaaatgattgccgccatagccttgttagtaaaagactctcacaaactaaccttggggcagcctttgaccatacatgcccctcatgcggttgaggcagtcatcagacagcctccagacagatggctcactaatgcccgaatgactcattaccagactatgctgttggacaaagaccgggtccactttggccctttggtgactctgaacccagccaccttgcccccctccctggggagcccgaggctcatgattgcttgcaggtactggctgaggcccatgggacaagacccgacctaactgaccagcctctacccagcccggaccacatctggttcacggatggaagcagctttttgcatcaaggagaacggagggctggtgcagcggtcacctcagaggatcaggtcgtctgggcccaggcactccctcccggaacctccgcgcagagggcggagctcattgcactcacgcaagctctaaagttggcagaaggtaagaggctcaccgtgtacacagacagtcgttatgccttcgccactgcccacatacatggagaaatttacagacggagggggtagcttacctcagaagggaaggacattaagaataaggaggaaatcctcgctctcctaagagctcttcatctgccctccgccttaagtatcatacattgccccggacatcaaaaaggggattctcttgaagcgaggggcaatcgaagggcagatttggccgcccaagaggcggccctggccacagaaaccactaacctcctggctctagaacccaccaacgaccgtccctccccctcatgggactatgaacaaagagacatccagaccctagagaaattgggagccaccaaggaatcaaacggggattggacttatgaaggaaagactatcatcccctaacgggtaaccaagtacctagtgacatttttacataagatgacacatctgagctccaagaagatgcgagaactcctcgagcgagaagacgaattcaatttccttttggggaagaataacattctaaaacaggtaactgaacaatgtgatgcgtgcgcccgagtcaacgcatccagactgaagcttcctcccgggaaccgggtcagaggctaccggcccggaacacattgggagatagatttcactgagattaaaccaggtaaatatggatacaagtatctattagtttttatagacaccttttcaggatgggttgaagccttccctactaaacatgaaacagccaagatcgttactaagaaattgcttgaagaaatctttccccgttatgggatgcctcaggtactggggacagacaatgggcccgccttcgtctcccaggtaagtcagtcagtggccaccttgttggggattgattggaaattacattgtgcttatagaccccaaagttcaggacaggtagaaaggatgaatagaacaattaaggagactttaacaaaattgtcgcttgcaactggcactagagactgggtcctcctactccccctagcactctaccgcgctcgcaatacccctgggccacatgggctcacaccttttgagatcctgcatggagtatctacccctatcattaacttccttgatcaagatgtctcagattttgctaactccccttctctccaagctcatttacaggccctccaacttgtacaacgggaggtctggaaaccccttgctcaagcttataaagaccagagggaccatcctaccattccccattcctaccagatcggggacaccgtctgggtccggcgtcaccagtccaagaacctcgaaccccgctggaagggaccctacatcgttttgcttaccacccccaccgcactcaaagtagacagcattgcagcttggatacatgcttctcatgtaaagccagcccgacccaccgattcagccactgcatcggaatggactgcgcactgcactcaaaatcctttaaagataagactctctcgtacaccctcctattgattgtttgtctgtttatcccccatgtagcaaccagcccctataccattcatgcgttaacctggcaggtactttcccagtcaggggaggtcatttatgagaccaccaaaaaccatgcccttggtgtatggtggcctacattgacacccgatttctgtgacttggtcgctggtctagacacctgggacatctcacactgttaccctaaggcttgtagcaacgaaaacgatttgccacgatttcgcaggtcactccaaagttcgagagacaccactcctggatgtagcagtcaacaggccaaagaccaactgtcgggtagtgacttttatgtctgtcccagagacggacgggcctggaagaagactcagacttgtgggggttatgaacagtttttctgtgccacttggggatgtgaaacgacaggggatgcttactggaagccttcatcctcatgggacaagattcaagtaaccagaggatggcaaaagggctccaccggcaccacctctatgcctctccttatttcctttacggatgagggtaagaaagcaacagaatggcagaacggatatgcttggggccttcgctggtacctgtcgggaccagaccggggggccatctttaaaattagactcaaagtaaaaactatcaccaaaaccatgggcccaaacccagttctggcagaccagaaacccccaccaagcctcatgggtctcggccccataggcctctatcccatctcccaccctcatcaacaccctcatccaatactgccacccttactcttacgactccagttcacacccccacctccccatatccagggacaagggacagattacttgaactaatacaaggggcttacttggccctcaattcctcagaccccaacaagactcaggaatgctggctatgcctagtttcccggcccccatattatgaaggcatagcagtactgggcaactactctaaccagacctcagcgcctaccagttgtggagctgccatgcagcacaagctcacaatgtctgaggtctcaggaaaggggctatgcataggcagggttcctccctcacatcaagagttatgtaaccaaacagagccattatctcaagacagccgataccttgttgccccttatggaacttattgggcttgcagtactggtttgacaccctgcgtctctaccactgtcctcaatgccaccattgacttttgtatattgacagaactttggcccaaagtcacataccaccaacctgagtatgtttacagcgtactagagaaatcaacccgacatcagagggagccaatgtccttaaccgtggccctattattaggaggaataacaatggggggtatagcagctggaagagggaccagtacggttgccttgcaggaaactagttatttcaaacttctacaacaagccatgcatacagatatccaggccctagaagagtcagtcagcgcactcgagaaatccttaacatcactctctgaggtagtcctgcagaacagacgagagttagatttattatttctacaggaacggggactatgtgctgccctcaaggaagaatgctgcttttatgcagaccacacaggaatagttagggacagcatggccaaacttagagaaagacttaagcagaggcaacaactatttgagtctcaacagggatggttcgaaggatggttcgctaagtccccctggttcactacccttatatccattctcatgggacctctgattattctatttttaatccttatatttggtacctgcattctaaataagatgactcaattcatcagagaacgactatctgtcatacaggccttagtcttaactcaacaataccatcagctaaagcaaatagatccagaatatccagagacctctgaatgaaagattccattcagttacaagagaaatgggggaatgaaagaccactgccccttaagctctctctctctctctcttgggctagctaaattacagaccaagagaccaagaccacccagaaccgtttctgagaatagaaggtcagggcgttatgactacttgtttgccacacaggatatctgtggtcagccatctggacgcggagggaggaactaccgggtctgttgtaaccactgtgtacccaagatagagccaagtcagcttcgtctgattaccagaccttgagaacttgctaacctgaaataactctgtgcctcatttgaattgtccaatagaatccctgtaactatgcttttcgcttctgtactatgctttttgctatataaggacccctctcccttggctcggtgcgcttggcctcacagaagctaagtcgccccgggtacccgtgtatccaataaagcctcttgctgtttgcatccaagttcgtggtctcgctgattcctgggtacgggtctccttctgtgaaagtacctcttctgggg

SEQ ID NO: 3 - DNA, 8181 bp, Cricetulus griseus
  misc_feature 1..8181: note = Intracisternal A-particule retrovirus like sequence of CH0-K1 cell
  source 1..8181: mol_type = genomic DNA
ctgtagagagctgcagaaaaccgcacccaaaagatggcgctggtttccgccttccgccagcccgacggcgagcgctctctgtggtaaacaactccaaatatggtagaggtcatgtatcctcttaattctgcttggagacaacctatcctggcgcgccacgtagggttaggtgattggtagatgtagactatatcaggccccgtctccctcggcctggggccgccgcctccattatacattgtacaaaagcaaagaggttcccgattaaactgtgtttgaagaagattcctcggtgtggcgtctttcttgctggtcaagggtggacgccgcaagtggtggcccgtacggggaattcagaacttctcacagtcggggcggcgccggtaagttcccaaggtaagtggaactgtaaagtccgcggtgaatgcggagataggtctccggtaaaggagcaccaaggtcctcaggaaagaggagataggtctcagtaaaagagtaccaaagccctcggggaagaggcagtaaagtctccggaaaggagcaacattgttcgcgacaaaagcgaaccgaaagtaaaaaaccctcgcttctgctttaatttgcagagtgaaagtaagttaatggaccccgctattatagtggtgatttggttgctttttaatatcgcagtgtgggcttttgttttctggtggtgttatccgacttgcacaccttcggagcgcgctaattaagacaaaagcacaatgggatcgtcacaatcacatccaatttttctggctcttcaagagctacttcattctaacaatttaaggttaactaaagccacattacagaggttcctgagtgaatgcgacaccattgcaccctggtttgcagcctcgggcaatctcactgtggatagttgggataaattagggaaagacctggattttgcctgggagcaaggagcgttgaagccgagtgttcgcccggtttggcgcctggtgcgcagctgtctagaggacaaagggggaaataagaaagctatagcaaatggacaggctgcccttgaaatgctccaagaagaaaggtcagaacagtcagaaagtgagagaacaaaggagaaacagaaaagggataagaagcgagtctatccttctctaggaaggttgaggaaggaattaaaagaaccaggagaggattcagaatcagaggaggatatatgtgaggatgaggacatcatggaggaagaggaggaagagattattgagctcatggaaaaacattcattaaaagtatcagaaaaacagcgtcctaaaatggcgactgtaaagcaagggcaccgccctatcgctcctccccctactgctcgaaagaggaagtaggaggtcctggttgttccaccttctgtccggaagtgtggcgtacggttcgcactgagtttcgggttactcgaccactggcttatccagtttttacagacggtaaccaacagagatatcatgaaccaattgactttaaaatagtcaaagctctggcagaatcggtacgcacctatggggtgacggcagcttttacaattgctcaggtagaggcgctcggtcgctactgtatgacacccagtgattggacaaatttggtaagagcttgcttatcaccaggacaatatttggattggagagcttttcaaattgagtttgccaatgatcaaggtgcgattaatcgtgcagctggtgatcaatattgggatgtggacatgcttcttggtcaaggacgttttgcacagcaacaaacaggatatcctcctcaagtgtatgatcagattaatcaaatagccattagggcttggaggtctctgcctaacaaaggtgaggtcagtggaaatcttacaaagatattacagggtcctatggaacccttttcagactttgtggcaagaatggtagaagctgcaggaagagtgtttggggatccagatgctgctatgcctttgatcaaacagctgatttatgaacagtgtacaaaagagtgtagagctgcaataactccatataagggtaaaggccttgaagtctggatgaaagtctgtagggagttagggggtccgctgactaatgctggactagcagctgctgtgatgcaattaactaagaaaggtggaggttcaggagcttgctttaaatgcggcaagcaagggcatttgaaaaagcaatgccccgagggaggaaacactaaagtcaataactttgctccgcgccctaagcaacctggcttatgtcctagatgtaaaaaaggaaatcattgggctaaggattgtagatcagtaaaagacatcagtggacagcctcttgttcaggggtatggaggagcccgttcaaaaaacggacgacggggcccacgaccccagggcccacaaatatatggggccatggaggatcagaaccaggagcagagtcccgaaacctggccctctcttcgtcatccgagggaccgaggagagccactacaggctccgcggggctggacttacgctccaccaccagactcgtattaactcccagaatgggggtccagcttgttgacaccgattttaagggaccccttgagcctggcacagtaggtttgcttataggaagatcatctgcagcattgaaaggtttacgagtacatcctggagttatagatcctgattacatgggtgtagtaaagatcatggtagaatctcctagagggattacggccatttctcctggagacaggatagcacagttactgcttttgccaagcttgcatgacaagtttccagcacaagccagagagagaggagagggaaactttggctccactggatcaaacttaactttcctagctttagaccttgatcaacgtccaacccttgagttaatagtgaatggtaagaaaatcttaggcttactagattctggagctgataagagcatcatagccactaaagattggccctctggctggcctatacaggtttcttctcaaagtttacaaggtttaggctatgctaaggctcctgatatgagtgctagacaattgccttggaaagatcaggaagggcattcagggaccatgcaaccttatgtgttagacttaccaatttcattatggggaagagatttgttaaaggatatgggttttaaactcacaaatgaatactcagaaacatctcaaggtatcatgaaacgaatgggatacgtcccaggccgaggcctcgggaaacatctgcagggtcgtaccagtcctattattccacaaccgagaccaaagaatctaggtctgggtttttcctaggggccactgagggaggtattcctattacctggaaaacagaggagccggtatgggttcctcagtggccactttcctctgaaaaactggaagctgctaagactctagtgcgggagcagctggatctggggcatataaaatcctctgtatctccatggaatactcctatttttgtcattaagaaaaaatctggtaaatggagactgcttcacgatcttagagctattaatcaacagatgcaaattatgggccctgtacaacgtggtcttccacttttaacttctttacctgcatcatggcctatcatctctatagatattaaagattgcttcttttccatacctttgtgtgccaaggattcagggcgttttgcgtttacgctgccctcttgtaatcatgaacaacctgatttaaggtatgaatggatagtgttgccacaggggatggccaatagtcctactatgtgtcagttgtttgtagcagaagcaattgctcctttgagagtggactttccaaagattagatgtgttcattatatggatgatattttattggctgccaaagatgataaaacgcttaataaggcatatacaaaattggtaaaattgcttgagatgcataatttagtcatagcctcagaaaaggtacaaaaggacactgttgttaactatctaggggctaagattctccctcatacaattattccacaaaagatagagattagaaaagataatttaaaaactcttaatgattttcaaaagttgttgggagatataaattggataagatgttatttaaaattaccaaattatgagttgaagccattgtataatattctcaatggtgattcagcattagattcacctaggcagttaactgctgaagccagagaagctttaaagaaagtggagacagaattacaaaatgccagccttaaaaggctaatagaaaatatggatattcttttgtgtgtgcttcctacattctcccagcctacagctttgctgtggcaagatggtccgttactatggatttattctaaatcctccccaggaaaaactattgaatactatcctgcagcggttgctgacttggctcaaaatgggatacagcaatgtattcaatattttgggaaattaccaattaagatcataatcccttatactgctcaacaagttaagatactttgtggaactgtggatgattgggcaattcttcaatgtggattttcaggagaaatagataatcattatccaaaacaccctctaatgtcattttttaaagaacatccagtcatttttcctaaaatgactgcagcagctcctattgctggagccgcaaatattttcactgatggatccaagacgggctgtggagcctacatgatagagcatcaggatccagtgcaatttcaatatcagcctggctccccccaaattattgaatgtaaaattgttcttgaagtgtttagaaattgcccgttctcctttaatctgatttctgattcggcatatgttgtgaatgctgttaaggccctggaggttgcaggacctattaaacctaatagtacagtttgcaaactcttacaggagttgcaaaagttgatctggcatcgcgatcagaaattttttatacaacatattagagctcataccaacttgcctggacctctatcaaaaggcaatgagatcgtagatctctgtactagaggggaatatgtatttttcgcttcctctatggaacgagcacagcactttcacaaacaatttcatgtatctgcaaaaactttacagcaaaggtttcagctatctcgagcagaagctagacagatagtattgaattgtcaacagtgtataaccttcttgcaccccctagcctgggggtcaatcccagagggttattgcccttgaagatctggcaaatggacgtaactcatttttcaggatttggaactcttaaatatatccatgtgtctgtggatacctgttcaggcatcatccatgccacccctatgagtggggaaaaggctcgcaatgtcattggacattgccttgaggcgtgggctgcctggggaaaacctcagcaattaaagactgacaatggtcctgcttatacagctcaatcttttacctccttctgtaaacagatggaagtccaattaaaccatggcctgccttataatccccaaggacaaggaatagttgaaagagcccatcgcacattgaaagaatgtttactaaaacaaaaaggggaataggccatggtagaacaccaaaggaacaactatctttggctctctttactctgaattttttaaatttggattctcaaggcctttctgctgccgataggcaccagactcgggcacctgctcagaagggctatgtaaaatggaaagatatccttacgggtctatggcatggaccggacccggtattagcatgggcgagaggttctgtttgtgttttccctcaggatcagcaagacccggtgtggattccagagagactaacgaggaggtgcaacaaacaagatgaagcctccgatattgatcctgctgctgatcttgacaacaccgtggctgatccaggccagagaacaaatcctgtgggccatagcgaaagcgtggccaattcctttgcctgttcatagcacatctaaagttttgcctgttttcttttctactagttgtgaattgggtttgccttgtgtcaatttagatcctgacactgcccagtattcagccactaatatttctctcaatggagcattatgcttctcccttattaataattctaacccatgcatttggttgaagaatggttctattggcaattggcttgatcctcttactaataaccagatctcgagtgctatgctcactgaagccctgtctcagtttagtacaggcgctagttctggctctggcactggtacaaatggaaccaaagatgtaaatatcactacatttctgatgctgatggaggggctccgaccctcaaattcacgctttagtcaagagaactccactgctcgacaagtcttgccttattgtgcacccaatcgaggctatccacctcattggacgccttgtcagagtccggatcacaaacttaagcaaatagctccaggttttgaatttactccccctcttggcaagtaccagtataacctaactcagggaggttcccaaagaacaggcagtaaaaacacctggccatggtttcaatgggtactctccaacgagcgtgaaacatttacttctcttgaaccttttgcagttctccagaatctctgcataagacttcttaatgtttctggtacaagggatgagaagggaccttctcttaagaatctatcaatacataaagctttgaataatctcactgttccagctagcccagtatgcttaaaagctccttttttctttctgctctctaatgctattgatgctaccaaagaagtgatctcgtgcaattcgactgatgtctcctgcattgctagccaatgttggaatggttctgctaatacagcagtagtgatgaggattcctatgtatgttcctattccagtcaaagtggatacaggaacgtttcccattacggaaatcatcagaaccaaacgggattttggcataactgcagcattggttactgccattaccctttctgcagcagcggctacgactgccaccgttgccatggctgctcaggtccaatcagctgagacagtcaatgatattgtggaaaaaacagctacaacattaactaccttgagatctattgatggccacttaaaagctggcattctaactgtgaaccaaagagtggacctgttgcaggaacaggtggatgatttagtaaccttaacttccataggatgcattcattccttttcttctttgtgtatcacctccaggatggccaataatttcacggagaacagcaacctgagttggcagctgagtgcctaccttcagggaaattggagtcaacagttcgagaacctgacagacaccctgacacaacagattattgccgtgaatgctacacgcctgagcctgcccactgttaataccctcttaactacccttaagcgagcctttagtttagttaaggaatgggctggaataggggttatgttttccatgatgctgttagccatttttgtttgcttgtggtgcctatgcaggatcaggaagtcgcaaaaatcccaagctgctatgttggtacaggcctttgctgctgtggaagcaggacagtcccctcaagcatggttatccatgctgacagacaaatagatgtctgtctcgcccttgctgagtgagtaatgcgtatctgaatcagtcatgctcaatgacgggcacctaccctcatgctgagcgaataagcatacgtgtgttgccttcaacctaagacatgagcctgtgagggcgacacagtgactctttgacgggtaagatagggacactggggttgaacctaagacagagatgactgaaatctgacaacagatagatactgctactcctataatattaaaacaaaaagggggaactgtagggagctgcagaaaaccgcacccaaaagatggcgccggtttccgccttccgccagcccgacggcgagcgctctctgtggtaaacaactccaaatatggtagaggtcatgtatcctcttaattctgcttggagacaacctatcctggcgcgccacgtagggttaggtgattggtagatgtagactatatcaggccccgtctccctcggcccggggccgccgcctccattatacattgtacaaagcaaagaggttcccgattaaactgtgtttgaagaagattcctcggtgtggcgtctttcttgctggtcaagggtggacgccg

SEQ ID NO: 4 - DNA, 8369 bp, Cricetulus griseus
  misc_feature 1..8369: note = Gibbon-ape leukemia virus like ERV of CHO-K1 cell
  source 1..8369: mol_type = genomic DNA
aaaaaaaaaaaaagaaaagaaaagaaaagaaaaaacagttcccagaataactacccgaggttctgatgacctctatggaatgtttcaacgccctaagtgtgcctcaggtgtatgattcatgtgacacatggtttatggtttaactgaagaatactgtatgactctgatatttccccattcttttgtgggttttttgcctttataagcctgttacaaattcagaccggggtcgaactcctctacccctgtgtggtgtatgggtttcgaccccagcatgctggtctgagatctcgttccgtttcactctcaataaacttcctcgtgtgattgcagcaagtctggtcttggtgcctactgggtgcgcgcctttcccgaggcttgagtgaggggtctcccttcgggggtctttcatttgggggctcgtccgggatttgcgcgaccacccaggggtcctagacccacttggaggtaaggattttcattcctcgttgttctgttgtgattctgtgtttgtggattgttcagagatctggtgcgatcgcagttctggtttttgcaaacgctctaatcgagtctgctcctccagaggggggagcagagtggacggggatagacgtatccgggtgtccaccgtttgttcgccctgggagacgtcccaggaggacaggggaaggaccagggacgcctggtggaccccacgaggaaagtccggggattagtttcctccttgacaatctgtagaccgtctgactcttttcaggatttctggtcgactcaaggtcggcgattttgtctctctgttttgttgtctttgtgattgttctttttgtgaagtgtgatagaaccaggaatgggacagaccgtgacgaccccgctaacactgaccctggaccattggacggacgttaagacgagggctcataatctttctgttgaagtcagaaaagggccatggcagactttctgctcctctgagtggccgagttttggcgtaggatggcctccggagggaacttttaacctgtctctcatttccgctgtcaagcgaattatctttcaggcctcagggggacaccctgatcaggttccatacattatcgtatgggaggacctggtccaaaatccgacaccctgggtgagaccctggacgacaggggcagaaacggtgacggtggccgtggccgccaaaccgaaagtaccccctccagataaaccggtacctgctccatcggcccctacgaagatttatcctgaaattgatgatggttcccttctcctcgactaccctccacccccgtatcctcaaccagcttcccacgcacgtcccccacaggtgtcgccccccgccgactcccggacggcctctgaaacaggaccggtggccggaacccgaagccgccggggtcgtagcccaggggagaaggcgcggggtttgactctgctactgccctacctttacgagcttatggacccgccccggctccgggagaactggtcccgctacagtactggccgttctcatcagccgatatgtataactggaaaactaaccactcctctttttcagagaatccctcaggcctgacagggcttcttgagtccctaatgttttctcatcagcccacctgggatgattgccagcagcttttgcaagtcctttttaccacggaagagagggagagaatcctcctagaagctagaaaaaatgttccgggaccagatggagcccccaccaacctccctaaccttatagatgcagcttttcccttgatccgccctgactgggattataactctgcagaaggtagggagcgtctcacggtctaccgccgggctctagtggcaggtctcaagggagctgcaaggcgacccacgaatttggctaaggtaagggaggtcctacaaggtccggtggagccaccctctgtctttttagagcgcttaatggaggcttacaggagatatactccatttgatccctcctcagagggacagtgggcggctgtagctatggcttttataggacaatcagcccctgatatcaagaagaagttacaaagattagaggggctccaagattatactttacaggatttagtgaaggaagcagagaaagtgtatcacaagagggagactgaggaagagaagcaggagagagaaaaaagagagacggaagaaagggagaaacggcgtgaccgccgccaagagaagaatctgactaggattttggccgcagtagtaggtgaagaaagggtagagaaaagacagttagggcacctgggcaacaaagcaaatagacccgtgggcggacagagaccacgacttgaaaaggaccaatgtgcatactgcaaagagaaaggacattgggccagagaatgccccaaaaagaaacagaagggccctaaggtactggcccttgaagaagattaggggagtcggggctcggtccccctccccgagcccagggtaactctctctgtggaggggaaccccgttgacttcttagtggacactggggctgaacactcggtcttgactgaaccattaggacaattaggatcaagaaggactctggtcacaggagccactggtagtaaactttatccctggacaacaaaaagaagtttaaaaataggaaaaagccaagtgactcactctttccttgtaatccctgagtgccctgcaccgcttctgggcagggacttgctgactaaactaaaggctcaggtccagttcacctcgacagggccggaagtcacctggggggagacacctatagcatgtttagtcctaaatctagaagaagaatatcgcctccatgaacccaagattgagaatttgccctccaaggaatggttggtagcttttccaggagtttgggcagagcaagcgggaatgggactggctaagcgggtaccgccagtggtggtcgaattaaaagcagctgctacccccatctcagtcaggcagtaccccatgactcaggaggctaaagaaggcattcgaccacacatccagaggctattacaacaaggtatactgattcattgccagtcccctggaatactcccctcttgccggtacgtaagcctggaactaatgactatcgacctgtccaggacttgagagaggtcaacaagcgggtactggacatccacccgacagtgcccaatccctacaatttgctaagttccctgccaccggagcggtcctggtacactgttctggatttaaaagatgcttttttctgcctcagactacatccaagtagccaactcctgtttgccttcgaatggcgggaccccgacggaggacacaccggtcagctgacctggaccaggcttccacagggattcaagaactcgccaactctcttcgatgaagcgctccactgtgaccttgcgcccttcagggcacgaaacccccaaatctctctcttacagtatgtagatgatttgctgcttgcagcctcgacctgggaactgtgccttaatgggaccaaagagcttctaaaagaattgggtgagttggggtaccgggtgtccgcaaaaaaggcccaattatgctgtccagaagttacctacctgggatacaccctccgagaagggaagcggtggctcactgaagcccgaaagaaaactgtgatgcagatcccgacccccaccactccacgacaagtacgtgagtttctgggaacggcgggcttttgtagactctggataccaggattcgccactttggcggcccctttgtaccctctgactaaagacaaagtcccatttacatggaaagaagaacatcagaaagcctttgaaaaaataaagactgccctgctcacagcccctgctttgactctgccagacttgaccaaaactttcaccttatacgttgatgaacgggctggaatagctcggggagtcctgactcaggccctgggtccttggaagcggccggtggcttacttgtcaaagaaactagacccggtagccagcggttggccctcttgtctgaaagccattgccgcagtagccttgcttgtcaaagatgctgacaagctgatcctgggtcaacatgtgactataatcgcccctcatgccttagagagtattgtacggcagccccccgaccgctggatgacaaactctcgaatgactcattaccagagcttgttactaactgatcgaataacttttgctacccctgctatcctcaaccctgccaccctactccctgaagcagatgactctactcctgtgcatcgatgcgctgatatcctggcggaggagactggagtcagaacggacctgactgatcaaccttggccaggtgtgcctagctggtatacggatggcagcagcttcgtggtggaaggaaaaagaaaagcaggagcagcggtggtagatgggaaacaggtgctgtgggccagcagcctcccagaggggacgtctgctcaaaaagccgaacttttggcactaattcaagctctgcgcctggcagaaggtaaggctgtcaacatctatactgatagccgctacgcctttgctaccgcccacattcatggagctatctataggcagagggcctactcacatcggcagggaaagacattaaaaacaaagaagaaatcctggctcttctagaagccatacacctacccaagaaattggccattgtccattgtccaggacatcaaaaggggactgacccagttacacggggaaaccagatggcagatcagacagccaagcaagctgcccatggaactacggtgctaatagaagaaatcagaaatcacccaccagagcccccggagcacacacataaaggctgtgccttgactgaagatgggcaaattgtcttaccagctaaagaagggcaggactatgtaaaaaggttacatcaactaactcacctcggaactgaaaaacttaaacaactaattaaaagttccaaatactgtgttcaagacctacacaccacaacaaaacaggtagtggaggcatgccaagcctgtactatgaccaatgcggcccgaccgttcaaggagcctggaaaaagaatgaggggagaccgaccaggagtctattgggaagtagactttactgaagttaagccaggaaaatatggtaataaatatcttctagtttttatagacactttttcaggatgggtggaagcctttcccacaaaatctgaaactgctcagatggtgaccaagaagatactagaagaaattctgcccagattcgggattcccaaggtaatcgggtctgataacggcccagcttttgttgcccaggtaagtcaagaattggccactcaactggggattaattggaaattacattgtgcttacagaccccagagctcaggacaggtagaaaggatgaatagaactctaaaagagacccttactaaattagctattgagaccggcggcaaggactgggtgaccctcctcccctttgcacttcttagagtccgaaactcgcccgggcgtttcggcctcaccccttatgaaatcctacatgggggaccgccccccttaacggagtcgtgcgggatattggatcctagcacttactccccctcctcttctgctttatttactcacttaaaggccttagaagttattaaaacacagatctggagtcgaattaaagaagcctacgctccaggaaccatcacggtgccccacgggttccaggtcggagactcagtcctggtcagacgacatcgcgctggcacgcttgagccccggtggaagggaccctacctggtgctgttgactaccccctcagcagtcaaggtcgacgggattgctgcctgggtccatgcttcccatgtcaagaagacacccagtcagggcaagaataaccatgaagaaaattggacagtggcaaccagtgacaatcctcttaagctgcgccttcgccgtagccccaaccttgggtaataacccccatgcccccaaaaactaacctgggaagtgcttaacgaagagggcgacgtcgtatggtcaacaaccgcagtgcatcccctttggacttggtggcctgatcttgcacccgatgtctgtaagttagcagccggctctatggcttgggatctttccagttacactgacttagaaaagccacccccagaagaacagtgtgtccctcatggcatagggagcacattggggtgttcaggacagtttttccgagccaacctacaggcagcagacttctatgtctgccccgggcagggacaatccagagaaatgaagagacggtgcggaggagctgcagactatttttgtaataaatggggatgtgagactacaggagaggcctactggaacccttcctcctcttgggatctaattatggttaagaagggtcgtaagccagaagagccagaaaaaggagaaagagatccctccaaatacccagcttataggactgggtgtgcctctggaagctcggatggccctcaggggccttgcaaaaacaattattgtaaccccttaaatatcagctttaccccacagggaaaacaggatcgtcaatgggtaagaggaaatcggtggggatggagagtctatactggcagcggggaccctggattcgtttttgtcattaagataaaaatagaaagtccacccacacgacccgtaggccccaattcggtgcttgataaccccatacccaggcctgagaggcctcctcctcccaccctgtcaccaaaggaaggctcatattctctctggactgcacccccacgtgtccctatggggactgaggaccttctgttcaatctgcttgatagtgcatttgttgttttaaatcgcactaaccctgctgctactcagtcctgctggctgtgttttacttccaatcccccttactatgaaggaatagctcagctacgtagctataatgttacttctgatcactcacattgcccctggggaagccaacgaaagttaaccctgtctgcagtgtcaggaactggactgtgtataggaaaagtacctaccaggcatcagcccctctgtaatgtaactatctccaacctgacaactggcccagataaatacttagtaccccctctcaatacctggtgggcttgtggtaccagcttgacccctgcctccatacttcactttttgacaatactaaagacttttgtattttagtccaattggtacctagggtcatttatcatgatgatgcctcgtttatagatgaatttgaccaccgtgctcgctacaagagagaacctataaccttgaccttagctgttttgttaggactcggagtggcggcaggaattggaacaggagcagctgccctcatccaacagccctattactataatgaactcagagcagctatggatgcggacttgggagccttagaacagtccataaccaaactggaagagtcattgacttcattatcagaggtagtattacagaacaggaggggattagacctgctttttctaaaagaaggagggctctgtgctgccctaaaagaagaatgttgcttttacgtagatcattctggtgtcataagagactctatggcaaagctcagagatagactagaaaccagaaaaaaagaaagagaaacacaccagggatggtatgaaaattggtttaaccggtcaccctggttctctacgctagtctccactttggtcgggcctttactcatattgttgctaatcctgacctttgggccgtgcatcctcaataaactagtagcctttgtaaaagaaagagtgagcgctgtgcaggtaatggtcctaagacaacaatatcaggtattacaagaagctgagggatcgctctaagattagagcttttacaaaaagaaaaaggagggaatgaagagtaaaaacaaaactttcaaagaacactagcccctgccctagggccctcttctctgtaggtaatcacacccggaactttcttaaaaaaaaaaaaaaaaaagaaaagaaaagaaaaaacagttcccagaataactacccgaggttctgatgacctctatggaatgtttcaacgccctaagtgtgcctcaggtgtatgattcatgtgacacatggtttatggtttaactgaagaatactgtatgactctgatatttccccattcttttgtgggttttttgcctttataagcctgttacaaattcagaccggggtcgaactcctctacccctgtgtggtgtatgggtttcgaccccagcatgctggtctgagatctcgttccgtttcactctcaagaaacttcctcgtgtgattgcagcaagtctggtcttggtgcctactgggtgcgcgcctttcccgaggcttgagtgaggggtctcccttcgggg

SEQ ID NO: 5 - DNA, 24 bp, Cricetulus griseus
  misc_feature 2..24: note = myristoylation target sequence 2 (Myr2), forward strand
  misc_feature 22..24: note = Protospacer adjacent motif
  source 1..24: mol_type = genomic DNA
gtcctaagcctagaaactatgggg

SEQ ID NO: 6 - DNA, 24 bp, Cricetulus griseus
  misc_feature 2..24: note = myristoylation target sequence 4 (Myr4), reverse strand
  misc_feature 22..24: note = Protospacer adjacent motif
  source 1..24: mol_type = genomic DNA
gcatagtttctaggcttaggaggg

SEQ ID NO: 7 - DNA, 23 bp, Cricetulus griseus
  misc_feature 1..23: note = myristoylation target sequence 8 (Myr8), reverse strand
  misc_feature 21..23: note = Protospacer adjacent motif
  source 1..23: mol_type = genomic DNA
gagtgttagggacaaaggagtgg

SEQ ID NO: 8 - DNA, 23 bp, Cricetulus griseus
  misc_feature 1..23: note = PPYP5 target, forward strand
  misc_feature 21..23: note = Protospacer adjacent motif
  source 1..23: mol_type = genomic DNA
gttggttgatctattaacggagg

SEQ ID NO: 9 - DNA, 23 bp, Cricetulus griseus
  misc_feature 1..23: note = PPYP6 target, forward strand
  misc_feature 21..23: note = Protospacer adjacent motif
  source 1..23: mol_type = genomic DNA
gccactgccgcccccaccagagg

SEQ ID NO: 10 - DNA, 23 bp, Cricetulus griseus
  misc_feature 1..23: note = PPYP7 target, forward strand
  misc_feature 21..23: note = Protospacer adjacent motif
  source 1..23: mol_type = genomic DNA
gcccccaccagaggcagaagcgg

SEQ ID NO: 11 - DNA, 23 bp, Cricetulus griseus
  misc_feature 1..23: note = PPYP13 target, reverse strand
  misc_feature 21..23: note = Protospacer adjacent motif
  source 1..23: mol_type = genomic DNA
ggcagtggcggatatggcggggg

SEQ ID NO: 12 - DNA, 23 bp, Cricetulus griseus
  misc_feature 1..23: note = PPYP20 target, reverse strand
  misc_feature 21..23: note = Protospacer adjacent motif
  source 1..23: mol_type = genomic DNA
gcttctgcctctggtgggggcgg

SEQ ID NO: 13 - DNA, 1020 bp, Cricetulus griseus
  misc_feature 1..1020: note = Rad51
  source 1..1020: mol_type = genomic DNA
atggctatgcagatgcagcttgaagcaaatgcagatacttcagtggaagaagaaagctttggtccacaacctatttcacggttagagcaatgtggcataagtgccaatgatgtgaagaaattagaagaagctggtttccatacggtggaggctgttgcttatgcaccaaagaaggaactcataaatattaagggaattagtgaagccaaagcagacaaaattctggctgaggccgctaaattagttccaatgggtttcaccactgcaactgaatttcaccaaaggcgttcagaaatcatacagattactactggctccaaagagcttgacaaactgcttcaaggtggaattgagactggatctatcacagagatgtttggagaattccgaactgggaagacacagatctgtcatacattggctgtaacatgccagcttcccattgatcgtggtggaggtgaaggaaaggccatgtacattgacaccgagggtacgtttaggccagaacggctgctagcagtggctgagaggtatggtctgtctggcagcgatgtcctagataatgtagcatatgctcgagggttcaacacagaccaccaaacccagctcctttatcaagcatcagccatgatggtagaatccagatatgcactgcttattgtagacagtgctactgccctctacagaacagactactcaggtcgaggagagctttcagccaggcaaatgcatttggccagatttctgaggatgctgctgcgacttgctgatgagtttggtgtagcagtggtaatcaccaaccaggtagtagcccaagtggatggagcagccatgttcactgcagatcccaaaaaacccattggaggaaacatcattgcccatgcatcaacaaccaggctgtacctgaggaaaggaagaggggagaccagaatctgcaaagtctatgactctccctgtctccctgaagctgaagccatgtttgccattaatgcagatggagtaggagatgccaaggactga

SEQ ID NO: 14 - DNA, 1053 bp, Cricetulus griseus
  misc_feature 1..1053: note = Rad51b
  source 1..1053: mol_type = genomic DNA
atgagcaacaagaaactaagacgagtaggtttatcacaagagctgtgtgaccgtttgagcagacatcagattgttaattgtcaggactttttaggtctttccccactggaacttatgaaagtgactggcctgagttatggaggtgtccaggagcttctgtatatggtcagcagggcctgtgccccacagatgcaaacagcctatgatataaagatgcggaggtctgctgaactctccccagcgttcctgtctactaccctttctgctttggacaaagccctgcatggtggtgtggcttgtggatcgctcacagagattacaggtccgccaggttgtgggaaaactcagttttgtataatgatgagtgttttagctacattgcccaccaacatgggaggattagaagggactgtcgtgtatattgacaccgagtctgcgtttactgctgaaagactggtcgagattgcagaatcccgttttccactctattttaacacagaagaaaaactgcttttgatgagcagtaaagttcatcttcaccgggaactcagctgtgaggcagttctgcaaaggcttgaatctttggaggaagagattatttccaaaggagttaagcttgtgattgttgactctattgcttctgtggtcagaaaggagttcgatcctcagcttcagggcaacatcaaagaaaggaacaagttcttggccaaacaagcatccttgctgaagtacctggctgaggaattttcactcccagttatcttgacgaatcaaattacgacccatctgagtggagccctcccttctcaagcagacctggtgtctccggctgatgatttgtccctgtctgaaggcacttctggatccagctgtgtggtagccgcactgggaaactcatggagtcactgtgtgaacacccggctgattctccagtaccttgactcagagagaaggcagattctcattgccaagtcacctctggctgccttcacctccttcatctataccatcaagggggaaggcctagttcttcaaggccaagaaaggccatag

SEQ ID NO: 15 - DNA, 1101 bp, Cricetulus griseus
  misc_feature 1..1101: note = Rad51c
  source 1..1101: mol_type = genomic DNA
atgcagcgggagctagtgagtttcccgctgtctcccacggtgcgagtgaagctggtggctgcgggtttccagacggccgaggacgtcctgggggtgaagccctctgagctcagcaaagaagttgggatatctaaagaggaagctttagaaactctacaaattgtaagaagagagagtctcacagacaaacccagatgtgctggtgcatctgtggcaggcaagaagtacaccgcactggaacttcttgagcaggagcacacccagggcttcataatcaccttctgctcagcgctagataacattcttgggggtggaatacccctaatgaaaacaacggaagtttgtggtgtaccaggtgttggaaaaacacagttatgtatgcagttggcagtggatgtgcagattccagagtgttttggaggagtggcaggtgaagcagtgtttattgatacggagggaagttttatggttgatagagtggtcacccttgcaaatgcctgcattcagcaccttcaccttatagcaggaacacacaaggatgaagaacaccagaaagccttggagggctttactcttgaaaatattctttctcatatttattatttccgttgtcatgattatactgagttgctggcacaagtctatctccttccagatttcctttcaaatcattcaaaggtgcagttagtgataatagatggcattgctcttccttttcgacatgaccttgatgatctatcccttcgtactcgattactaaatggccttgcccaacaaatgatcagcctggcaaataatcacagattagctgttattttaactaatcagatgacaacaaagattgataaaaatcaagcattgcttgttcctgcattaggggaaagctggggacatgctgctacaataagacttatttttcactgggagcaaaagcaaaggtttgcaacattgtacaagtcaccaagccagaaggagtccacaataccatttcagatcacaccacagggatttagagatgctgctgtcactgcttcttcatcacagacagaaggttcttcaaatctccggaaacggtcacgagaaccagaggaaggatgctga

SEQ ID NO: 16 - DNA, 1002 bp, Cricetulus griseus
  misc_feature 1..1002: note = Rad51d
  source 1..1002: mol_type = genomic DNA
aaacatgggcgtgctcagggtggggctgtgcccgggcctcaccgaagagatcgtccagcttctgaagggccaaaggatcaagacagtggcggacctggcagctgctaacctggaggaggtagcccagaagtgtggcttgtcctacaaggccctggttgccctgaggagggtattgctggcgcagttctcggctttcccattaaacggagcagacctatatgaggaactgaagacttccactgccatcctgtccacaggcattggaagcctggacaaactacttgatgctggcctctatacaggggaggtgactgaaattgtaggaggtccaggtagcggaaaaacccaggtgtgcctgtgtgtggctgcaaatgtggcccatagcctgcagcagaatgtactgtatgttgattccagtggaggaatgacagcatcccgcctcctgcagctactacaggctagaacccaagatgaggagaaacaggcaggtgctctccagaggatacaggtggtgcatgtatttgacatcttccagatgctggatatgctacaggaccttcggggctccatggcccagcagtcgacatcttcttcaggcactgtgaaggttgtgattgtggattctgtcactgccgtgattgccccacttctgggaggtcagcagagggaaggcctggccttgatgatgcagctggcccgagagctcaagatcctggcccgggacctagctgtggcagtggtggtgaccaaccacttgacccgagacagggatggtaggaggttcaaacctgctctgggacgctcctggagctttgtgcccagtacccggattctcctggatgtcaccaaaggggctggaacattaggcagaggccaacgcacagtgtgtctgaccaagtctccccgccagccaacaggtctacaggaggtgatagacattgggacattggggactgaggagcagagcccagaattgcctgccaaacagacatgatgctgttt

SEQ ID NO: 17 - DNA, 1257 bp, Cricetulus griseus
  misc_feature 1..1257: note = Rad52
  source 1..1257: mol_type = genomic DNA
atggctggagctgaggaaacagtccgtggaggctgtgacacccatcctccctttgctggtgggaaatctgtgctgtgctttgggcagagccagtacacagcagaggaataccaggctatccagagggctctgaggcagcggctgggcccggagtacatcagcagccgcatggctggaggaggccagaaggtgtgttatattgaaggtcatcgagtaattaacctggccaatgagatgtttggttacaatggctgggcacactccatcacccagcagaatgtggattttgttgacttcaacaatggcaaattctacgtgggagtctgtgcatttgtaagggtgcagttaaaggatggttcctatcatgaggacgtgggttatggagttagtgagggcctaaggtcaaaggccttgtcactggagaaggccaggaaggaggctgtgactgacgggctgaagcgggcactcaggagttttggaaatgcacttggaaactgcattctggacaaagactatctgaggtcactaaataagcttccacgacagctccctcttgaagtggatttaactaaagcaaagagagaagattttgaaccatctgtggaacaggcaagatataatagctgcctacagaatgaagcaccgggacccccaaaaccacaagaagcggcttccccttgcagaccaagccacccacatgattcgaacattaggctgcagggggctaaggactgcagcagctcctgcagtctggccgcccccatggagagtgatgccattcaccagcgcaagctccggaagctccggcagaaacagctgcagcagcagttccggcagcagatggaggcccacctacagggccacacacctgccgtaaaagtgaaagccgagcgtgaggcagtgcttccagaccttcctccaaaacacagtacccctgtaactgctgcctcagaactcctcagggagaaagccatttttccagataaccctgaagacaaccttgaaatgtgggacctgactccggatttagaggacatcattaagcccttgtctagaccagaaccacctcaaacctctgccaccagagtccaggtgatccaggatggtgtcctacacggcctttgccaccagatgccaccagaaaaacatgaagctggtcacctgcaggcccacagcactcaccagcatgtattaggaaactctgactctcataggaagagccaggacctgaagaaaaggaaactggatccatcctga

SEQ ID NO: 18 - DNA, 2235 bp, Cricetulus griseus
  misc_feature 1..2235: note = Rad54
  source 1..2235: mol_type = genomic DNA
atgaggaggagcttagctcccagccagttggccaggaggaaaccagaaggcagatcatctgatgatgaagactggcagcctgggacagtaactcctaagaaacaaaaatccagtaatgagacccagtgcttcctgtctccttttcggaaacctttgactcagttaatcaaccgaccgccttgtctggatagcagtcaacatgaagcgtttattcgaagcattttgtcaaagcctttcaaggtccccattccaaattatcaaggtcctctgggctgtcgagcattgggcttgaaaaaggctggtattcgtcgtgccctccatgaccctctggaagaaggtgccttggttctgtatgagcctcccccactaagcgtccatgaccaactgaagctggacaaggagaaactccctgtccacgtggttgttgatcctattctcagtaaggtgttgcggcctcatcagagagagggagtgaagttcctatgggagtgtgtcaccagtcgtcgaatccctggaagccatggctgtatcatggctgatgagatgggcctgggaaagacactacagtgcatcacattgatgtggacacttttacgccagagcccagagtgcaagccagaaattgagaaagcagtggtagtgtcaccttccagcttggtgaagaactggtacaatgaggttggaaagtggcttggaggcaggatccaacctctggccatcgacggaggctctaaggacgagatagaccgaaaactggaaggattcatgaaccagcgtggagcgagagtgccttctcctattctcatcatttcctatgagacttttcgccttcatgtcggagtccttaaaaaaggaaatgttggactggtcatatgtgatgagggacacaggcttaaaaactctgagaatcagacttaccaggccctggacagcttgaataccagtcgtcgggtgctcatctctgggacccccatccaaaatgacttgcttgaatatttcagcttggtgcacttcgttaattcaggcattttgggaactgcccaggagttcaagaagcattttgagctgccaattttgaggagtcgagatgcagctgccagcgaggcagacaggcagctaggggaggaacgtcttcgagagctcatcagtatagtgaataggtgcctgatacggagaacatcagatatcctctctaaatatctgccagtgaagattgagcaggtggtttgttgtaggctgacaccccttcaaactgagttatataagagatttctgagacaggctaagcctgaagaagaattgcgtgaaggcaagatgagcgtgtcttccctgtcttctatcacctctctaaagaagctatgtaaccatccagccctaatctatgacaagtgtgtgtcaggggaagatggctttgaggatactttggatatcttcccacctggttatacttctaaagctgtagaaccacagctttcaggtaaaatgctggtccttgattacattctggccatgactcgaagccgcagcagtgataaagttgtgctggtgtctaattatactcaaacgttggatctctttgaaaagctgtgccgagctcgaaggtacttgtatgttcgcctggatggtacaatgtccattaagaagcgagccaaggttgtggagcgcttcaatagtccatcgagtcccgattttgtcttcatgctgagcagcaaagctgggggctgtggacttaatctcattggtgctaaccggctggtcatgtttgatcctgactggaacccagccaatgatgaacaagctatggcccgagtctggcgtgatggtcaaaagaagacctgctatatttaccgactgctatctgctggaaccatagaagagaagatctttcagcggcagagccataagaaggcactgagcagctgtgtggttgatgaggagcaagatgtagagagacatttctctcttggcgagctcaaagagctgtttaccctggatgaagctagcctcagcgacacacatgacaggctgcattgccgccgttgtgtaaacagacgccaggtctggccaccccctgacggttctgactgtacttcagatctggctcagtggaaccatagcacagataaacggggactccaggatgaggtactccaggctgcctgggatgcttcacctacagccatcaccttcgtcttccatcatcgttctcatgaggagcagcggggtctccactga

SEQ ID NO: 19 - DNA, 837 bp, Cricetulus griseus
  misc_feature 1..837: note = Xrcc2
  source 1..837: mol_type = genomic DNA
atgtgcagcgactttcgtaaggccgagtccgggacggagctccttgcccggcttgaaggcagaagctccctgaaagaactagaacccaacctgtttgctgatgaagattcaccagtacatggtgatgttcttgaatttcatggtccagaaggaacaggaaaaacagaaatgctttatcatttaacagcccgatgtatacttccaaaatcagagggtggactgcaaatagaagtcttatttattgacacagattaccactttgacatgctccggcttgtgacagtgctcgagcacagactgtctcgaagctccgaggagaccatcaagctctgcctgggaagattgttcctggcctactgcagcagcagcttgcagctactgctcacgctgcactcactggaagccctgttctgtagtcacccctctctctgccttctcattgtggatagcctgtcggctttttactggacagaccgcgccagtggaggagagagtgtggccctgcaggaatccactctgaagaagtgttctcagctcctagagaggcttgtcactgagtaccgcttggtgcttttcacaacaacacaaagtctaatgcagaaagcctctgactcagcggagcagcctgcttcctccaagctcccaggtgacggagacacagactacagagcctatctctgcaaggcctggcagaaggtggtgaagcacagagtcatcttctccagagaggacgaggctaagagcagccgcttctcattagtttcacgtcatttaaaaagtaacagtttaaaaaaacatgcttttatgatcagagaaagtggggtggaattctgttga

SEQ ID NO: 20 - DNA, 1050 bp, Cricetulus griseus
  misc_feature 1..1050: note = Xrcc3
  source 1..1050: mol_type = genomic DNA
atggacttggatcaactggacctaaatcccagaattactgctgcaattaagaaggggagactgaggtcagtgaaggaggttctgtgctactcgggaccagacctgcagaggctcaccagcctgtccacccacgatgtgcagcacctactgagagtggccgctctgcacctccagggcagccgggtcctcacagcactgcagctgttccagcagagggagagcttccccgagcagcatcaacgcctgagcctgggctgcccggtcttggatcagttcctgggtggcggcctgcccctggagggcatcactgacctggctggtcgaagctctgcagggaagacccagctggggctacagctctgcttgactgtgcagttcccacgacaatatggaggcctggaggctggggctgtctacatctgcacagaggatgccttccccagcaagcggctgtggcagctcattgaacagcaacagcagctgcggacggatgttcctggggaggtgatccagaagatcagattcagcaaccacatcttcatcgagcatgcggccgacgtggacgccttgctggagtgtgtgagcaagagggttcccattctgctgtcaagggggatggcccgcctggtagtggttgactctgttgctgccccattccgttgtgagtatgatgctcaggccttggccaccagggccaagcacctgcagtctctgggagccgcgctccgcagactgagcagtaccttccggagccctgtgctgtgcatcaaccaggtgatggaaacggtggaggagcaagagtctatgcccaggccactgggggcctgggatgagcacctctctccagcccttggcatcacctgggccaaccagatcctgatgagactgatggttgaccgggcacatgaggacgatgcctccatgggcttacccagaagcccggcacggaccatacgggtgctctctgccccgcacctgcccctctcctcctgctgctacacggtcagtgcggaaggcatcagagggataccgggaaccgagtcctgctaa

SEQ ID NO: 21 - DNA, 5373 bp, Cricetulus griseus
  misc_feature 1..5373: note = Brca1
  source 1..5373: mol_type = genomic DNA
atggatttatctgctgttcaaatcgaagaagtacaaaatgtccttcatgctatgcagaaaatcctggagtgtccaatctgtttggaattgatcaaagaacccatttccacaaagtgtgaccacatattttgcaaattttgtatgctgaaacttcttaaccagaagaaagggccttcacaatgtcctttgtgtaagaatgagataaccaaaaggagcctgcaaggaagcacgaggtttagtcagcttgttgaagagctgttgaaaatcactgatgcttttgagcttgacacaggaatacagtatgcaaacggttacagtatttcaaaattgaaaaattcttctgaacctttgaatgaggaagcttccatcatccagagtgtgggctaccgaaaccgcggcaaaagacttagacagattgaatctggaaatgccaccttgaaggacagtctcagtgtccagctgtctaaccttggaattgtgagatcaacaaagaaaaatcatcagacacagcctcgaaataaatctgtgtacattgaattagagtctgattcttctgaagagacagttagtaaaccagatgactgcagtgtgagagaccaggaactgttacaaaccacctctctgggagctggagatgaagtcagtttggattcttcaaccagagctgcttgtgagttttctgaggacgtaacaaatattgatctgcaccagtgcagtaataaagatttggaccccattgagaatcatgcaactggaagatatccagaaaaatgtcaggatatttctgtttcaaacttacatgtggagccatgtggcacagatattcatgccagctcgttacagcatgagaacagcagcttattactcactgaagacagaatgaatgtagaaaaggctgagttctataataaaagcaaacagtctggcttagccaggagccaacagaacagatgggctgaaagtaaagaaacatgtaatgataggcagattcccagcactgagaaaaaggtagatccaaatactgattccttctgtgggagaaaaaaatggaaaaatcagaaaagtctgtgtcctgagaattctagagctacccaggatgttccttggatgacactgaatagcagcattcagaaagtcaacgagtggttttccaaaactggtgaaatgctaacttgtgatggcacatctgacaggaggcatgagtcaaatgctggagcagctgttgtgttagaagtttcagatgaagtagatggatgttccagttcttcaaaagaaatggacttagtggcctctgatccccataatgctttaaagtgtaaaagtgaaagcgacctctccaaaccagtagagaacaatatccgagataaaatatttgggaaaacgtatcagagaaagggaagtctccctcacttgaaccacgtaactgaaattataggcacatttattgcagaaccacagataacacaagaacaccccttcacaaataaattaaaacgtaaaaggagaactacatgccttcatcctgaggattttatcaagaaagcagatttaacagttactcaaaagacttctaaaaatgtaaatcaaggaactgaccaaatggagccagatggcctagtgatgggtattaccagtaatggtcaagagaatgaaacacaaggtaataatcttcagaaagagaaaaatgctaatccagtgaagtccttggaaaaggggtctgtttccacaactaaagccaaaactataagcaacagtataagtgatttggagctagaattaaatgtccacaattcacaggcacctaagaaaagtcggctgaggaggaagtcttcaaccagatgtgttcttgcactggaaccagtcagtaggaatccaagcccacctgcttgtactgaatttcaaattgatagttgtagcagcagtgaagaaacagagaaaaacaattccaacccaacaccagtcaagaacattagaaagcctcaactcacggaggacacagaacctgcagcagattccaagaagaataaagagccaaatgaacaaagaaggaagagaagggccagtgatcctttcccagaagagaaatttatgcctggcttattaactaactgtccaagttctagtaaacctcaaggacctgccaatcctagccctcagagaaaggaagtagagaaacttgaaacaagccaaatgtctgacagtaccaaagacctgagggatctgttgctggatggagaacagggtttgcccactgagaggtcggaggagagtaccagtgtttcattggtgcctgacactgattatgacactcagaacagtgtgtcattgctggaagctaatgctgtcagatatgcaaaaacagtatcaagccagtgtatgacccagtttgtagcaagtgataaccctaacgaactggtccatggttctaaagatgctggaagtggcacagagtgcttcaagcatccattgagacacgaacttagccacattcaggagaccatagaaatggaagagagtgaacttgatactcagtatttacagaatacatttcaagcttcaaagcgtcagtcatttactttattttcaaaaccaagagatccccaagaggagtgtgtaacagcttgtgcttcctctgtgtccttaagggacatgagtccaaaagtgacttctgaaggtgaacaacaagaagaaagtcggggacacgaagaggctgaaatcagtcacatacaggcagggcctgcgacagtgggcgtgcctctgcctcgtcaggaaggtgatccaggcgctgatacaatgcatgctgcagtgtgcagcctttgtccatcatctcagtacagaagcaatgaaaactcacatcctaaacaatcagcttctcccatcatgtcttctataaaaactggccatgggaaaaccctgtcagaggaacaatctgagaaacatacattgtcaaatgaaaaggcaatgggaaatgagacctttgttcaaagcacaatgcacacaattagccaaaaccacagaaaaaatggttgtcaagaagccaactcaggcagtattaatgaagtgccttccagtggtgaaaacttccaaggacagctaggccgaaacagaaggactaagttaaacactgtgcttccactaggtcttatgcaacctggagtctgtaagcatagttttcctgtcagtgattataaatatcttgaaataaaaaagcaggaaggtgagaatgttggtgcacacttctcttcgtgtctgttttcagataagctcgaacaacctgtagggagtggtaatgtttttcagatttgttctgagacacctgatgacctgttggatgatgttgaaatacaggaaaatactagctttggtgaagttgacataatggagaagtctgctgtttttaacgggagtgtccagagacgagagctcagtaggagccctagccctttaacccatgcatcgctggctcggaatctccagagacggtctaggaagttagagtcctcagaagacagcggatcttgtgaggatgaagaccttccctgcttccaacacttacttggccaagtaagcaaaacacctgaacctagcagtgttgtgacacagcctctgtcagagaaagcagaggggacccaagtgccatggaagagtagtgttggtgactctgataacgaggtgatcttgatagaggcatctcaggaacatcaccctagtgaggatgcaaaatactctggcagcatgttctcttctcagcccagtgctgtacaaggttcaactgcaaatacaagctcccaggaccccttgtttaacctttccaaacaaaagagtcaccagtctgaggatgaggaagattttctaagtgacaaggaattgatttcagatgatgaggaaatgggaacttgcctagaagaagataatgatcaagaagaggatattataatcccagattcagctgaagcggcatctggatatgagagtgaaacaaacctttctgaagattgctcacagagtgatattttaaccactcagcagagggccaccatgaaggataacctgataaagctccagcaggagatggcccacctggaggctgtgctagaacagcaaggggaccagccttctgtccactcctcgtctctcacagctgaaccttgtgcccctgaagacctaccaaacgcagaacaaaacttatcaggaacagcaattttaacttcaaagaatattaatgaaaatcctgtaagccggaatccagcatgcgtttctgctgacaagttccaaccacaacctccagacagttccagcagtgaaaataaagagtcaagggaaggaaggccttccctttttaaatctccgttggcaggcagtaggtgctctgcacacagccgctctgggagtcttcaaaacagaaactgcccatctcaagaggagctcctttggactgttgaagcagggaagtcagaaggaacgccatacctggaatctggaatcagccttttctctaatagagaccctgaatctgagtcccctaaagagccagcttatgtttgcaccacaccagcttcaacctctgcacggacaatatcccagtatcaggtttctgactctttcgagagtccggctgctactcatgctcatactgcagtgatagaaactgtgagcaagaaaaagccagaattgacatcttcaaaaggaagagccaataaaggaatatccatggtggtgtcaggcttgacccccaaagaagtaatgattgtgcaaaagtttgctgaaaaataccgcctcactttaactgatgcaattactgaggagactacccatgtcattatgaaaacagatgctgagcttgtgtgtgaacggacactgaaatattttctgggaattgcaggaggaaaatggatagttagctattcatgggtgattcagtctattcaagaaagaaaacttctgagtgtgcatgaatttgaagtcagaggagatgttgtgactggaagaaaccaccaaggtccaaagcgatccagagaatcccaggaaaagctcttcaaaggcctaacaatctgttgttgtgagcccttcaccaacatgcccaaagatgagctggagaagatgctgcagctgtgtggggcttccgtggtgaaggagctttcgtcactcacccctgacaccggtgttcatccaattgtgattgtacagccaagtgcctggacagaagagaatggctgccctgagattgaccagctgtgtggggcgcacctggtgatgtgggactgggtgttggacagtatatccgtctaccggtgtcgggatctggacgcctacctggtacagaatatcacccatggccacgacagcagcgagccacaggaccccaatgattag

SEQ ID NO: 22 - DNA, 10098 bp, Cricetulus griseus
  misc_feature 1..10098: note = Brca2
  source 1..10098: mol_type = genomic DNA
atgtccaatgaatacaaaaggagaccaactttttttgaaatttttaaggcacgatgcagcacagcagacttaggacctataagcctgaattggtttgaagaactttcttcagaagccccaccatataattttgagcctccagaagaatatgaatataagaccaacaattatgagccacagctgtttaaaacgccacagaggaagctctcttaccatctgttggcttcaactccaataatattcaatgaacaaggtcaaactctaccagtagaccagtcgcctttcaaagaactagggaagactcttggaaatagtagacataaaaatcaccacagaaccaaggccaaaatggactccatggttgatgttgccagtccacctctgaagtcttgtctcagtgaaagccctcttactctgcgatgcacacaggttgtaccacaaagagaaaagccagtgatgtgtggaagtttattttctacaccaaaaatcgaggagggtcagacacctaaacctatttctgaaagtctgggagttgaggtggatcctgatatgtcttggtccagttcattagctacaccaccaacccttagttccactgtgctcatagcccgagatggggaagcacgaggaattgtgtttcctgaagactcccccgctgttttgaaaagctacttttccagccacaatgaaagtctgaaaaagagtggtatatcagttccctctgtgactgacagcgagaacaaaaaccagagggaagcttttagtcatggattggggaaaacgttagaggattcatctggcaaaacaaacagcttcaaagattgccttaggcagtcaataccaaatgttctagaagatggggagacagctgcagatacttctgaagaagatagtttctcattatgttttcctaagcataaaaccagaaaaatgcaaaaaatgagaatgggcaagaccaggaagaaaattttcagtgaaataagaactgatgaattaagtgaagaaactagaagtggagctgatggaaaacattcatttgcacttgaaattgacccaagagatagtgatcccttagatccagatgtgacaaaccagaaatgctttgacaatgggaatgaggaaatctgcgaggaagttgtacagtcttcagacactcgatggtctcagctaaccctttctcgtctaactggtacccagaagggaaaaatacctctacctcctatttcttcttgtaaccaaaataattcagaaaaagacttcatagatacgaaggaagaaggtattgactctgttactttagaaaattctttgcctcatatttctagttttccagaaccagaaaagatgttctgtgagaaaactctggtagataaggaacatgagggacagcatcttgaatcacatgagaactccattgcagggaagcaagtggtatctggaacttgtcaagcagcttgcctattgagagagccacttgaagagtctctgggtaatctcttctcagagagtgtgactagctcagcctttacagaagaacccagtgcctctgcaagtggattgggaatatgtgctgtgtcctcacagagagaggattctttgtgtcctagttcaggtgacactggaaactggccatcaactctcactcacacttctgcaactgtgaacaatacaggtttaatatccagtctaaaaactaaaagaagaaagtttatttactctgtcggtgacagtgcatctcatcaaggaaaaatactacaggcagacagaaagtcagagctcactaacacttctgctcagttcgaaacaagtgcttttgaagcgccattcacgtttacaaatgggaattcaggtttatcagattcttctctccaaagaagctgtttacagaatgatgctgaagagccatctttgtccttatccacctcatttgtgactgcttccaagaaagaaagtagttatagtaatgcattgatatctcaggatctccatgacaaagaagcaatagtcagtgaagaaaaactgcagccacatacagccctggaaactgattgtctgtcgtgcttgccagaaagacaatgtgaatatgatccaaagggtcccaaagtttcagatggaaaagaagaagtcttagtctcagcatgtcatcctgcaggacagcacacagcagcagcacagcccagcagcattagctttgaattacaggaagaccctgtcaatggccacaatagtacaagtcctaaagaaactcctagcttgaaggtgcttctgtcaaagccagttgtgctttctagaggaaaagtgtcatgtaaaatgccagagaaactgcagtgtgagagttataaagataatactgaattaagcaaaagcattcccttgggaggaaataaaatacacatcctaagtgaaaattctaaacctcctgagcttctgccacctggaaaatatgtaacagaagcatcccccacagtgaagtctcagttcaatcagaatacaaatctagcagtcaaaaaaaatgaccaagaagaaacgccttttatttcagaagtaacagtcaatgtgagttctggagaacttttcccagacaatgagaataatttcgcctttcaagtaactcatgaaaataataagactgccttaggaagtactgtggaactgcaggaagaagacctccgccatgctaaagggcctaatctcaacaactctcccacagcagtagatggagacataggtgatgagcaagcagctcatgcactgattatggaagactccgattcctcagctttagtccatgagtgtgcaaagaagagcagaaatactatagagcagcatctgaaaggaaccacagacaaagatttcaattcctccttggatgtgaaatcagatgggaacaatgactatacagacaaatggccaggatttttggatccagtttttaagcataaatttggaggtagcttcagaacagcttccaataaagaaataaaactttcagaacataatgttaagaaaagcaaaatgttcttcaaagatattgaagaggagtatcctactagtttaacctgtattgacatagttaatgcctcaccattagcaaaccaggagatactaagtggaccttatacatttgatttgcagtcagtcactaccatgtctgcccatccacagagtcaggcatctgtttcttgtgaagatactcacacatcacttcaggtgttaccttcaaagcaagattttcattcgaatcacaatttaacacccagccaaaaggcagaaattacagaactttctactatcttggaagaatcaggaagtcagtttgaattcacacagttcaaaaagccaagccatgtagcacagaataatatacctgaagtgcctggaaaacagacggttgccataaatactacttctgaggggtggaaaaggattggtcttcatctcacagtggatcctgcctctgtagctcagacagatgacagcaagaaatttgaaggttctgctggatttagacaaagctttccttgcctgttgaaaagcagttgtaacaaaaatacatctagttttttagcaaatgtaaatgaaatggagtttcgaggatttcgttctgctcttggcacaaagcttagtgtgtctagtgaggctctgcaaaaagctgtgaaactcttcagtgacattgaaagtggtagtgaggagacttccacaaaagtcgacccaagagctttgtcttcaggtgctcgtcatgattctggtgcttctgtgtttaagataaggaaacaaaacagtggtaaaagttttgatgagaaaactagtaagtgccaagtaacattacagaataataccgaagtgactactggtatttttgttgacagaaatcctgaaaattatgcaagaaatacaaaatgtgaagataacaactctactggttttcaaagaagtccttataaattaaaaaactcggaggatagtgaatcaagtacaagcggcacagtttctgttcatcaagatgatggtgacttaccatgtgctgctgatcactgcagcaagtaccctgagtcgtgttcccaatatgtaagggaggaaaacacacaaattaaggaatgtgtatcagatttaacatgtttggaagtcatgaaagctgaggaaacatgctatattcaaccttcagataaagaacaattaccttcaggtaagatggaacaaaatagaaaagattttaatatatcctttcagactgcaagtgggaaaaatgtcagagtctctgaggagtcattaagtaaaagtatgaatattttaaatcaagtaacagatgaatcgatcatctcttcagattctttgaattctaaatttcattgtggcacaaataacaacaagatgggtatttcacatcacaaggaaactaccagtactaaaaaggtatttgaagaacgtttcccagttgggactgtcagtcaattaccaactctccagcagcatcctagatgtgaaatagaaagtatcaaagaacctgctctgttgggttttcataccgctagtggaaaaaaagtcaaaattatgcagaaatctttagacaaagtgaaaaacctttttgatgagacacagtatgttagtcatcaagggtcaaaacccttgaaggacagagagaactgcaaagaaggacttgcattaggatgtgagacaattgaaatacctgcctccaagtgtgaagaaatgcagaagagctttctctctaaggagtctgaagtgctatctaagcaaagtgatcgtttgtataggtcgactgaaaatctcagaacatcaaatggtacctcttctaaagctaatgtacatggaaacatagaaagtgaaatagaaaaaagtcctacaacttgctgcattagtcatttatcttattcagtcactgaagattctgctttgacatgtgacacaggacacggtagaaaaacttgtgtcagtgagtcttctctatccaaagacagaaaatggcttagagaccgactgggtgataagcttcaaaaaagagatgctgccgaaattgaatgtgtaaaagaacatactgagggttatgctggagatgcctcatgtgagcatagtttagacagtatcagaaatgaagttgatataaattgtgtctctgaaaatcaaacttcagccctctttagtgaccctagcatgtgtcatagctgtccatcccattttggttgtcattgtgataacaagcataatgactcaggatatttctcaaaaaataaaatttattctgatactcagccagacacgaagaatgaagacactgccaatttttccagtgtatatgctacaaaagaagtaaatatatacccgccaactgtaaatgaagatatttgtgttcagaaacttgagactaactcttcaccacatacaaataaaaatgtagccattgacttggctatagcagattcaaggaattgtaaggtatgcccatccaagttcattacagatcactcacaagaaactgtgaaaacagtaaaagcaatatttacacataacagtgataaaacaattaagcaaaacacaaagagtaaaccagatacttgccggacaagctgtcagaaagcattggataattcagaggattttatatgtcctagctctttagaagatgactatatgaactcacataagacttctgtttatacccacgatgaacaaatattacagcataacctaagtgtgtctggactggagaaagctcaaataccacctgttcacttggaaacttgggataagtgtaaatctacaagggaacttgcacaggcagcctgttcttcacacatgccggggatttttagcacagcaagtggaaaagctgtacaggtatcagacgcttcattagaaaaggcaaggcaagtgttttctgagatggatggtggtgctaaacagttactttccacattgtctctggaaagtcatgaacaatcagaccactctgggagaagagaaaactctgtgacacataaccctgaggatgtattgtcactcccaaaaacctttgcaagcaatgccaattcatctgtattctctggatttagtacagcaggtggtaaacgggtcacagtttcagaaagtgccttacacaaagttaagggaatgttagaggaatttgatttgatcggaactgaacatactctccagtgtccacctacatctgaaggtgtatcaaaaatacttcctcaatattgtgttgaaaagagaaccccagaataccctataaactctaaattgcagaaaacctatgatgataaattcagtttaccaaacaactataaagaaagtgcttctttggggaatactcattctcttgaagcttctccccaactctctcagtttaagcaagacacacggttggtattaggaaccaaagtatcccttctggaaaaagaacaaaccttccctcaaaatataaaaacagaaagtggtgtaatggaaacttgtcctgatgtccctgtgagaacaaatgtaggagactgttctgctttcgacaaaaagccagagaactgttttgaaacagaagcggtggagattgccaaagcttttatggaagatgatgagctgacggattctgaaccaagtcacgacaaatactcattgtttacctgcccccaaaatgaggctttgttaaatttgagaactagaaagagaagaggaatggctgttgatgcagttggacaacccccaatcaaaagaagcttattaaatgagtttgacaggataatagaaaataaaggaaaatccttaaagccttcaaaaagtgctccagatggtacaataaaagacagaagattgttcacgcaccacatttctctagagccagttacctgtggacccttctgctctaccagagaaaggcaagaaatgcagagtccacatttcactgcacctgctcaaggacttctgtctaaagggcatccttctgtgcgttcggctttggaaaaatcttcaagcaattctacagtttcagtccagccgactcataaagtttctgctacaaggaatgaaaggacaggatgctcagtcacaggcaaatcccacaaagtcttcgtcccacctttcaaaatgaaatcacagtttcacagagataaacatttgaataacaagaatattaattcagaggaaaagaaccaaaagagcagagatggagacagtgaagatgtgaacgacggtgacattcgtcaatttaaaaaaggcagcttccatcaagaggccgctagaattgtcacagagtgtgaagaagagcctttagatttaatgacaagccttcagaatgccagagacctacagaatatacgaattaaaaagaaagaaaggcatcagctctgtccacagccaggcagtctgtatcttaccaagtcatccactctgcctcggatctctctgcaggccgcagtaggaggccggcttccctctgcgtgttctcataagcagctctatatgtatggcgtttctaaagagtgcataagcattaacagcaaaaatgcggaatattttcagtttgacattcaggattattttggtaaagaggacctgtgtgctggaaagggcattcagctggctgatggtggatggctgatcccctccaatgaagggaaggctggaaaagaagaattctatagggctctgtgtgacactccaggtgtggatccaaagcttatttctagcctctgggtctctaatcactaccggtggattgtgtggaaactggcagctatggaatttgcttttcctaaggaatttgctaacagatgcctaaccccagaaagagtgctgcttcaactaaaatacagatacgatgtggaaattgacaacagcagcagatcagccctaaagaagatcctggaaagggatgatactgctgcaaaaacactcgttctctgtgtttctgacgtcatgtcactaagcacaactggatcagaaacttcaggcggcaaaactagtggtgcagatcccaagagcatagacacgatcgaactcacagacggatggtatgctgtcaaggcccagctagaccctccacttgtggctcttgtaaagagcaggagactgactgtgggtcagaaaatcattacccaaggggcagaactggtgggatctcctgatgcctgcgcacctctggaagcaccagactctcttagactgaagatttctgccaacagcacacggcctgctcgctggcacagcaaactggggttctttcatgaccccaggcctttccctctgcccttgtcctcactgttcggtgatggaggaagcgttggctgtgtggacatcattgttcagagagtgtaccctttacagtgggtggagaagacggcatcaggattgtacatattccgtaatgagagagaagaggagaaggaagcagtgagatttgcagcggcccaacagaagaaactggaggccttgttcaccaaaatccaggcagagtttaaagaccatgaagaagactcagcgcagcggcgcgtgctgtcccatgcactcacaaggcagcaggtctatgctctgcaagatggtgcagagctctatgcagcggtgcagagtgcattggatccagatcaccttgagggttgtttcagtgaagagcagctgagagccttgaacaaacacagacaaatgttgaatgataagaagcaagcacagatccaatcagaattccggaaggccgtggagtccgctgagcaagaggagggcttatcaagggatgtttcaactgtgtggaagcttcgagttacaagctacgagagaaaagaaaagtcagctctgctgagtatctggcgtccatcatcagacttacactccctgttaacagaaggaaagagatacagaatctatcatctggcagtatcaaaatctaggagtacatttgaacgacctagcatccagttaacagctacgaaaagaactcagtatcagcagctaccggctgccagtgaaaccgtattccaggtttaccagccgagggagccgcttgacttcagcagactgttagagacagcctttcagcctccttgttctgaagtggacctcgtaggagctgtagtttctgttgtaagaacaacaggtcttgctcctttggtctacctatcagacgaatgccttaatttattagtggtaaagtttgggatagaccttaatgaagacataaagccacatgtgctaattgctgcaagcaacctccagtggcacccagaatccagatcaggagtgccaacgttatttgccgggaatctttccatagtttctgccagtccaaaggaggtctacttccagaagcgagtcaacagaatgaggcaaactgttgagaatatcgatacattttacaaggaggcagaaaagaagcttttacatctgcttaatggaaacagtcccaagtggtccaccccaaataaagactctactcagccggcctacacttgccctgcttcagagctccttgctacaggaggtcagctaatgaggttctcacctaatagtgagcaaagttatccaagtcctttatcacattgcacaccaaaagaaaagtctacacccctggctcggtcagcccagatggcatcaaagtcttgtaacagggagagagagactgatgaccgaaaaacctgcagaaaaagaagagccttggattttctgagtcggctgcccttacctcccccggtcagtcccatttgtacctttgtctctccggctgcacagaaggcttttcagccgccacggagttgtggcaccaaatatgcaacacctatgaagaaaaaagaacccagttcccctcagaggaggacgccatttcagaaggacagtggcatttctcttctggaacaagattcggtagctgatgaagaacttgccttcctcaatacccaagctcttgtacctggctcaccagaagaaaatcaacaagtacttcctggtgactccacaagaacctctgtgccccacaacaaaagaacccccagccagcacagaggccagaccagcatgtaggaccccgatccaggaaggggagtctatctcagggattacagaggtggcaagggcagtgatggaaaattagctgtcgagtcttag

SEQ ID NO: 23 - DNA, 888 bp, Cricetulus griseus
  misc_feature 1..888: note = Cyclin D1
  source 1..888: mol_type = genomic DNA
atggaacaccagctcctgtgctgcgaagtggagaccatccgccgcgcgtaccctgacaccaacctcctgaacgaccgagtgctgcgagccatgctcaagaccgaggagacctgcgcgccctccgtgtcttacttcaagtgcgtgcagagggagattgtgccgtccatgcggaaaatcgtggccacctggatgctggaggtctgcgaggagcagaagtgcgaagaggaggtcttccctctggccatgaactacctggaccgtttcctgtctctggagcccctaaaaaagagccgcctgcagctgctaggggccacctgcatgttcgtggcctccaagatgaaggagaccattcctttgaccgccgagaagttgtgcatctacactgacaactctatccggcccgaggagctgctgcaaatggaactccttctggtgaacaaacttaagtggaacctggccgccatgactccccacgatttcatcgaacacttcctctccaaaatgccagaggcggatgagaacaagcagatcatccgcaagcatgcgcagacctttgtggccctctgtgccacagacgtgaagttcatttccaacccgccctccatggtggctgccgggagcgtggtggctgcaatgcaaggcctgaacctgggcagccccaacaactacctgtcctgctaccgcacaacgcactttctttccagagtaatcaagtgtgacccggactgcctccgcgcctgccaggaacagattgaggcccttctggagtccagcctgcgccaggcccagcagaacatcaaccccaaggccaccgaggaagagggagaagcagagggagagactgacctggcctgcacacccaccgatgtacgagatgtggacatctga

SEQ ID NO: 24 - DNA, 882 bp, Cricetulus griseus
  misc_feature 1..882: note = Ercc1
  source 1..882: mol_type = genomic DNA
atggaccttgggaaagacgagggaagcctgccgcagcccaccaggaagaagtttgtgatcccactggaagacgaggcccctcctgcaggggccaagcccttattcagatcctcacggaaccccagcaccacggccccctcggtcccagcggcccctcagacgtacgccgagtatgccattgcccagcctccaggaggggctgggcccacagggcccacaggctctgaacctgtgaagggagagaaccccggccagacggtgaaaacgggagcgaaatccaatagcatccttgtgagcccccggcagaggggcaaccctgtgttgaagttcgtgcgcaacgtgccctgggaattcggcgaggtgacccctgactatgtgctgggacagagcacttgcgcccttttcctcagcctccgctaccacaatctccatccagactacatccacgaacggctgcagagcctggggaagagctttgccctgcgtgtgctgttggtccaagtggatgtgaaagatcctcagaaggccctgaaggacctggctaaaatgtgtatcttagcggactgcaccctggtcctggcctggagtgccgaggaagcaggacggtacctggagacctacaaggcatatgagcagaagcccgctgacctcctcatggagaagctggagcagaacttcctgtcccgggccaccgagtgtctgaccaccgtgaagtcagtcaacaaaaccgacagccagaccctcctggctacatttggatcccttgaacagctcttgacggcatcacgggaggacctagccttgtgccccggcctgggcccccagaaggcccgcaggctctttgacgtcctccatgaacccttcctcaaagtgccccgatga

SEQ ID NO: 25 - DNA, 4473 bp, Cricetulus griseus
  misc_feature 1..4473: note = MDC1
  source 1..4473: mol_type = genomic DNA
atggaaaacacccaggttattgactgggatgctgaagaggaggaggagacagaaatacccagtgggtccttggggtatagcttggagcctatagggcagctgcgtctcttcagtagtactcatggaccagaaagagatttcccactctaccttggcaagaatgtaattggccgaagccccgactgctctgtggccctgccttttccatccatctctaaacagcatgcagtaattgaaatctcagcttgcaacaaagcccctatcctccaggattgtgggagcctcaatggcactcaactggtaaagcccccaaaggtcctaacccctggagtgagccatcgtctgaaggaccaggaattaattctgtttgcagactttccctgccagtaccgtcgcttggatgtccctccacctttggtctctcggggacttctaactatagagaagacccccagaatacagggagggtcccaaacctccagagttttgttggctgaggattcggaggaggaagtagattttccttctggaaggtatgtggcaaatggatcaaggaatccaacgtctccatcagcaacagtagtaccagaaagtgatgaagaggggtcttctccagcccaaagtgtccgtgggccgtctttgccctttgacttgggcagtgacacagatgaagagctatgtccacagccagcagttggggagtcctcttcagttgccagggatggtgctgctgcagaggccaagcagctggaagctaatggagtgacatctggtatccagctggtactggctcagcctgctgagcaaaagctcagagatgcaaaagtcaagagtgaggctggcaatggagcggctgtggttgggaggggctccactgtgggtaaggacagcaacacggaaatgggtgaagaacaccagccttccggctttgtggacagcgatacagacgtggaagaagacaggatccctgtgaccccccctgtagttccggggaagaagcaagtcctgcttggagttggtaaaaaggaccctggagcacctgtcgtgacccatctgcaggacagcccagctggtcgtggcacaggtgtggaagaaggcaagactccagtggctggccctccagagagaaaccacacggccatggtgatcagcagcgacacagatgaggaagatggagtctcagcagcagtcaccttggcccatctgaaagacagggggatggctctgtggagcagggagccaggcatggaagaggtcaagtcccaaccacaggtcctcgtagaacgaagccagagtgcctctgggaaagacagtgacacagacgtggaggaggaaaagagagaagtggtccctgacagccccatggacacagatgaggctcttactgtcccacattcagagagccaagctccccatagagccagtgatgatgtagaccagggtgtggatatgggctcccctggtggtcagctagagggaaaccaggcctcctctgccacagtagaagacagtagtgcacaagcagggaaggaagtcctgcctcctgaggaggcctgggaaacagctgtggaggaaggctcatcttcagcagcggcagatgtaagacaaagcaagcagccaggagtagaagatgctgggacagagtgggctgcaactgtttgtgagcaggcgagcatgcttgaggtgggggtccaaggcaggtcacctgctacactggtagagccagtggtggtgcctacagctactctgggggatcccactcagccacagagagagggagcccagacccccacaggaaaggagagaggagtacaaatgggcaggaccaagaatgccaaagactgccgtgatgcagagtctgaagatgtgtgccttccggctacccagtgctttgtagacagggagggccagagctcagaagctgtccagagtttggaagatgagcctacgcaagtctttccatgcactcttccccaagagcctgggccttcccacctcagcctgcagacgccaggtccaggtgcccaggatgtgccttgggaagtcttagctacacagccgttttgtctgagagaagctgaggcctctgaactgcagctcatggacacccaccctgcagctcatgaatcccacccatctgtgtctagtgcatcagcaggacagcagcatctggttcacacagagccgctgggaattgaaggcggagagatgcaaactgtggagaaagccatgggccaattgagttgcaagatggcatctgctggagaggactcaaggggtgatccagaaccctcggcccatcgcctgctttctccagttcctgaagcttcttctccacctcagagtctgctcacctctcagagccaaaagccgtctacaccccagtctctgttacttacctctcccccttctgagctacaccttcctgaaactcctcacaccaagcctaatgtcaggcctcggcggtcctccaggatgacctcctccccacactcctctgctgcccttaagccctatactacctgccccacaaacctgcctgctgcccctagaccaacatctcgggctactcggggcaggggcagggcaaataggtcctctaccaggaccccggaaccagttgtccctacagactctgagcttcaacctcccacctccacagaacagtctgtcatccccaaacccacatctccagtcactcagggcagcataaatggttcctttgttaaaacgcctgaaccagttgttctcacaggtcccaaaatccagcctcccacctccacagaacagcctgttaccccaaaccccacacctcaggccactcggggcaggccacataggtcttccatccagaccccagaaccacttactcccactggtcctgacctccagcctcccacctccacagaacagcctgtcatacctaaacccacatctcgggctgctcggggcaggtcacgtaagtcttctaccaggaccccagaaccagctgtccccactggtcctgacctccagcctcccacctccacagaacagcctgtcacacctaaacccacatctcgggccactcggggcaggtcacgtaagtctgtcagaaccccagaaccagctgtccccactggtcctgacctccagcttcccacctccacagctctgggcactttgggaagggcatgtaagtcctccattgaggattctgaatcagttggaccagtagcctctgattttgaacctcccatctccacagaccttgttgcccctgaggtgacaggtcagagcataacactaaagtcttcaccactaagtgcttctccagtttctgccacctctgaactccagccacctgtccccacagcccagcctgttctcctggagcccattcctcaagccaaccaccgaaggcggcggaaggctgctgggaaacggggctcccacacagttcccattggccaaaagccttactctgcaccctctgaacctggttcccaatcttcaatcaatcaaggcttggcattggaagccgctgagtctattactgttgctcctgagcctgctgtttcccaggctccagagacctccactcagaatcccgtggtgcaaaatgaagcagctgggagatcagggctcattcccaagccccagcctgaggtttctcgatcccgcaagaagccttctactaccacaacctcaccaattcaaaaacgtccccgaagacaaataccccagaagacaatagtccccaaggaagaagatcctgaggaaatgccagtgaaggaagagcctcaggagatagcaattccaacacccggcaaaagaaagagggaccgtgtagaggatgagacccagggaaacccaagtcggagccggcgggctaaacctaaccaagaagcagcagcccccaaggtactgttcacaggagttgtggattctcggggagagcgtgcagtcctggctctgggtggcagtctagccagctcagtaaatgaggcctcccacttggtcactgatcgcatccgaagaacggtcaagttcttgtgtgccctggggaagggcatccctatcctctccctgaactggctgtatcagtccagaaaggctggtcacttcctgccacctgatgactacttggtgactgaccctgaacaagagaagaactttagcttcagccttcgggactccctgagccgggctcggcaacaaaaactgctggagggctatgagatttacgtgacccctggagtgcagccacccccacctcagatgggcgagatcatcagctgctgtgggggcaccaacctgcccagcatgccccagtcctataagccttaccgagttgtcataacttgcactgaagacctacctcgctgcgctatcccatctcgactggggctgcccctcctctctcctgagttcctcctgactggagtgctaaagcaggaagtcacaccagaggcctttgtcctttcaaattag

SEQ ID NO: 26 - DNA, 2412 bp, Cricetulus griseus
  misc_feature 1..2412: note = Bard1
  source 1..2412: mol_type = genomic DNA
atgcaactttctgagtctgtggttggactactaggtaggtacacaagccctgctcgggaacgaaacccacgaggagccccgccccttggcgttgagggcgtggccttgggccgcgctcctctcagaaattcgagcgcgctctcattgcctctcccctccctcttacttgctataaaggagcaacccaggatggtctgctgtctgcttcggctcatcatcatccagagtgtcgagttctttgaaatcatcactaatattctgaaggagccagtatgcttaggagggtgtgagcacatcttctgtagtgtgtgtataagtgattgcgttggatcaggatgtcccgtgtgttacacaccagcctggatcctagacctcaagataaacagacaattggacaacatgattcagctttataggaagcttcaaaatttgctacatgacaataaactttcagatccaaaagacaacacatctaggacaggttcatttgatgatgcagaaagcaagaagaattcaataaaaatgtggtttagtcctcgaagtaagaaggttagatgtgttgtgaataaagtatcagtgccaacccagcctcaaaagacaaaagatgacaaagcccaggaagcctccatatacgaatttgtttccacaagtccccctgaagttgtttctaagagggctaaaacagcttctagaacatctgcaaaaaagcagcagaagaaatctttagctgagatcaacaagaaggggaattcaaggccagaaacagaagatagcaggtttgattctaaagaggaactgaaagaggagagggttgtctcctgtagccaaataccagttatggaaagtccacaggtaaatggtgaaatagacttgttagctagtggttctgttgcagaatctgaatgttctggaagattgaccgaagtttctttaccattggctgagcatatagtatctccaggcactgtgagcaagagtgaagagactcctgagaagaaagtctgtgtaaaagatcttctttcagtagggcgtaatgaaaatcacaaatactgtagcaggcctcctgatcctacttctaagaattgtgagagaagcattccgagcaccagcagagatgtcattaaaccaacagtgcttgcagaaaatatactgttggttgactgttcttcactgccttcagaccagcttcaagttgatgtcacactcaggagaaagagtaacatatcagataactcccttagcctttcaccaggcacacccccaccactgctgaataattcaactcccagacaaatgatgtcaaaaccctccatagtgaagctgtcacccagcattaccgccaggaaaagaaaccacagaggagagactctgcttcatattgcttctattaagggcgatataccttctgttgaatacctcttgcaaaacggaaacgaccccaatgttaaagaccatgctggatggacaccattgcatgaagcctgcagtcatgggcacctgaaggtagtggaattgctgctccagcataacgctttggtgaataccacaggctatcagaatgacacaccactgcacgatgcagtcaagaacggccatgtggatatagtcagggtgttactgtccaatggcgcctccaggaatgctgttaacatatttggtgtacggcctgtggattatacagacaatgaaaatataagatcattattgctgctaccagagaagagtgaatcatcctcaactagccagtgtgcagttgtgactgctggacagcgaagaaatgggcctctggtgcttataggcagtgggctttcttcccaacagcagaaaatgctcagcaaacttgagacagttctcaaggctaaaagatgtgctgagtttgacagtacagtaactcatgtcattgttcctgatgatgaagctcaaagtactctgaagtgtatgcttgggattctcaatgggtgttggatcctgaagtttgactgggtgaaagcttgtttgcacagcaaagtacgtgaacaggaagaaaagtacgaagttcctggaggtccgcagaagagcaggctcaacagagagcagctgttgccaaagctatttgatggatgctacttctttttggggggaaacttcaaacatcatccaaagaatgatctccttaagctcattactgcagcaggaggcaaggtgctcagtagaaagcccaagccagacagtgacgtgactcagaccatcaacactgttgcataccatgccaaccctgactctgaccagcgcttctgtacacagtacatcgtctatgaagatctgtttaattgtcgcccagaaagggttcggcagggcaaagtctggatggctccttccacttggcttatcagctgtgtgatggcctttgaattgcttcctcttgacagctga

SEQ ID NO: 27 - DNA, 2796 bp, Cricetulus griseus
  misc_feature 1..2796: note = Lig1 (Ligase 1)
  source 1..2796: mol_type = genomic DNA
atgagaaaaaaagaaccagagaggaaaggggagaactctgctgccaccatgcagaaaagtatcaggtcatttttccaacccatgaaagagggtaaagcacagaagccggagaaggagacagctaacagcaccaaagagaaggagccacctccaaaggtggcactgaaggagaggaatcgagcagtgcctgagagtgattctccagtgaagaggcctggaaggaaggcagcccaggtcctaagcagcgaaggggaggaggaagatgaagcccccagcacccctaaagtccagaagtctgtgtcagactccaaacaaagctctcctcccagccctgacgcatgtcctgagaacagtcctttccacagtagcccctccatggagatctccccatcaggattcccgaagcgtcgcactgctcggaagcagctcccgaaacggacaattgaggacactgtggaggagcagaatgaggacaaaggcagagcagccaagaaaaggaagaaggaagaagaagcacagactccaatggaaagcctcacagagagtgaagatgtaaaacccaaggaagaaaaggaggagggcaagcatgctgaggcttccaagtcccctgagtcgggaaccttgacaaagacagagaccatcccagtgtgtaaggccggcgtgaaacagaagcctcaggaagaggagcagagcaagcctcctgccagaggcgccaagacactcagcagcttcttcactccccggaagccagcagagaaagccatagtgaaacaagaagagccaggtactccagggaaggaagagaccaagggagccctggatccaacaaattacaatccttccaagagaaactaccaccccattgaagatgcctgctggaaacatggccagaaagtcccttttctcgctgtggcccggacctttgagaagattgaggaggtttctgctcggctcaagatggtggagacactgagcaacttgctgcgctcggtggtggccctgtcacctccagacctgcttcctgttctttacctcagcctcaaccgccttgggccacctcagcagggactagagctgggtgttggtgatggtgtcctccttaaggcagttgcccaggccacaggccgtcagctggagtccatccgggctgaggtagctgagaagggtgacgtgggactggtggccgagaacagccgcagcactcagagactcatgctgccccctcctccgctcaccacctccggggtctttaccaaattctgtgacattgcccggctcactggcagtgcttccatggccaagaagttggatgtcatcaagggcctgtttgttgcctgccgtcactcggaagcccggttcattgccaggtccctaagtggacgcctgcgcctcgggctggctgagcagtccgtcttggctgcccttgccctggctgtgagcctcacaccccctggccaagaatttcccccagctgttgtggatgctgggaagggcaagaccacagaggccagaaagacatggttggaagaacaaggcatgatcttgaagcagaccttctgtgaggtacctgacctggaccgaatcatcccggtgctgctggaacatggcctggaacgcctcccagagcactgcaggctgagcccaggggtccctcttaaaccaatgctggctcatcccactcggggtgtcagcgaggtactgaaacgctttgaggaggtggactttacctgcgagtacaaatatgacgggcagcgggcccagattcatgttctggaaggtggagaggtgaagatcttcagtaggaaccaggaagacaacacaggaaagtacccggacatcatcagccgcatccccaagattaaactcccctcggtcacctcctttatcctggacactgaggctgtggcctgggaccgggaaaagaagcagatccagccattccaagtgctcaccacacgcaagcgcaaggaggttgacgcctcggagatacaggtgcaggtgtgtctgtatgcctttgatctcatctacctcaacggagagtccctgattcgccagcccctgtctcgacgtcggcagctgctccgggagaactttgtggagacagagggtgagtttgtcttcgccacctccctggacaccaaggacatcgagcagatcgctgagttcttggagcagtccgtgaaggactcctgtgagggactgatggtgaagaccctggatgttgatgccacctatgagattgccaagaggtctcacaactggctcaagctaaagaaggactaccttgacggtgtgggcgacactctggaccttgtggtgattggcgcctacctgggccgggggaagcgtgccggccggtatgggggcttcctcttggctgcctatgatgaggagagtgaagagctgcaggccatatgcaagctgggaactggattcagtgatgaagagctggaggagcatcaccagagcctaaaggccctagtgttgccgaccccacgcccctatgtgaggatcgatggggcagttgccccagaccactggctggacccaaaggtcgtatgggaggtgaagtgtgcggatctctccctgtcccctatctaccctgctgcgcggggcctggtggacaaagagaaagggatctcccttcgtttccctcggttcattcgtgtccgtgaagacaagcagccagagcaggccaccaccagtgaccaggtggcctgtttgtaccggaagcagagtcagatacagaaccagcacaactcagacttggactccgactttgaggactgctattaa

SEQ ID NO: 28 - DNA, 2121 bp, Cricetulus griseus
  misc_feature 1..2121: note = Mre11
  source 1..2121: mol_type = genomic DNA
atgagccctgcagatccacttgatgatgaagacacttttaaaatcctggttgccactgatattcaccttggatttatggagaaagatgcagttcgaggaaatgatacatttgtgacacttaatgaaattttaaaacttgccctggaaaatgaagtggattttattttgttaggtggtgatcttttccatgaaaacaagccctcaaggaaaactcttcatagttgcttggagttgcttaggaagtactgtatgggtgatcgccctgtgcagtttgagatcatcagtgaccagtcagtcaactttggttttagtaggtttccatgggtgaactatcaggatggcaatctcaacatttccattccagtgtttagtatccatggcaaccatgacgatcccacaggggcagatgccctctgtgccctggatattttaagctgtgctgggtttgtgaatcactttggacggtcaatgtctgtggagaagattgacattagtccagttctgctgcagaaaggaagcacaaaacttgctctgtacggcctagggtccattcctgatgaaaggctctatcggatgtttgtcaataaaaaagtaacaatgctgagaccaaaggaagatgagaactcatggtttaacttatttgtgattcatcagaacaggagtaaacatggaagtaccaacttcatcccagaacagtttttggatgacttcattgacctcgttatctggggccatgaacacgagtgtaaaattggcccaaccaaaaatgagcagcagctcttctatgtgtctcagcctggaagctcagtggtgacttctctttcccctggagaagctgttaagaaacacgtgggcttgctgcgcgttaaagggaggaagatgaacctgcagaagctgcctctccgcacagtgcggcagttcttcatggaggatgtggttctcgctaaccacccaaacctgttcaaccctgacaatcctaaggtgacccaggccatccagagcttctgcttggagaagattgaagaaatgcttgaaaatgccgagcgcgaacggctagggaattctcttcaaccagagaagcctcttatccgactacgggtggactacagtggaggctttgaaccttttagtgttcttcgctttagccagaaatttgtggatcgggtcgctaaccccaaagatatcatccactttttcaggcatagggagcaaaagggaaaaacaggtgaagagatcaactttgggaagcttgtttcaaaatctccttcggaaggaacgacactcagagtagaagacctggtgaagcagtatttccagactgcggagaagaatgttcagctctcactgctgacagaaagagggatgggtgaagccgttcaagaatttgtggacaaggaagaaaaagatgccatcgaggaattagtgaagtaccagctggagaaaacacagcggtttcttaaggagcgccatatcgatgctctggaagacaagattgacgaagaggtccggcgtttcagagaaagcagacagagaaataccaacgaagaagacgatgaagtgcgagaggccatgagcagggcccgggcgctcagatcgcagtcagagaactctgcctcagccttcagtgctgacgacctgagtttcgatatagcagaacagacagcaaatgactctgatgacagcctgtcagcagtgcccagcagaggccggggccgaggccgaggtcgaagaggaggcagagggcagagcaccgcatcaagaggaggatctcagagaggccgagacactgggcaggagacggctactcgaggcagatgctcaaaggccactacatcgacctctagaaatatgtccattctagacgctttcagatctactcgacagcagccttccagaaacactgccactaaaaattactcagagactattgaagtggatgaatctgatgaagatgacatttttcctaccagttccaaggctgatcaaaggtggccaggcacaacatctagcaaacggatgtcccagagccagatagccaagggggttgactttgaatcagatgaggacgatgacgatgaccctttcatgagcagtagttctctaagaagccgaagataataa

SEQ ID NO: 29 - DNA, 5973 bp, Cricetulus griseus
  misc_feature 1..5973: note = 53BP1
  source 1..5973: mol_type = genomic DNA
atgccaggggagcagatggaccctactggaagtcagttggattcagatttctctcagcaagatactccttgcctgataattgaagattctcagcctgaaagccaggttctggaagaagatgcgggctctcacttcagtgtgctgtctcgacaccttcccaatctgcagatacacaaagagaaccctgtgttggatattgtgtccaatccggaacaagcagctgcagaagagcaaggagacaataatagctccttcaatgaacatctgaaagagaacaaagctgctgcagatcctgtggattccactcatttgggcacatgtgattccatcagtcaggtcatagaacagttgcctcagccaaacaggacaagcagtgttctgggagtgacagtggaagctgcttctcttccagaagaggagaaggaggaagagctggaagaggagaatgaagaggtgggagcagatgctgctgatgccgctccgtgctcccttggtgctgaagattctgcttcatcacagttgggctttggggttctggaactgtcccagagccaggatgttgaagaacatacagtgccatatgatgtcaaccaagaacatatgcagttggttaccaccaactcgggttcttcccagctgtctgacatggatgcaaataatgttaaatgtgaagaacagtccactgaagatacctccatggcagaacaacctaacaaagacatccctgttacagtcgagcacagtaaaggtatccctgtggtagatgagcaaaatctaccaccggcaaggccagaggatctgccttccagtcctcaagcctctgctgcagctgtggaaacaaaggaagaggtacctgcccaagagttgccagaaggggcgctggaggttcagatgtcctcggagcctgaggtctcgtccactcaggaggacttgtttgagcagagtagtaaaacagcttctgatggttgttctactccttcaagggaggaaggtgggtgttctccggtttccacacccgccaccaccctgcaactcctgcagctgtctggtcagaggccccttgtccaggacagtctttccacgaattcctcagatcttgttgctccttcccctgatgctttccgacctaccccttttatcgttcctagcagtcccacagagcaaggagggagaaaagttgagcccttggatatgtcagtgatgcctgaagaaggaggggagactttgcagaagcttcaggatgacgaagcagtggagatagaaaagccccatctcccatctcagccggctgtttccccacaagtgtcaacaccagtgtctcagagcacacctgtcttcactccaggctctcttcccatcccgtcccagcctgaattttctcatgacattttcattccatcaccaagtttggaagaaccatcaagtgatgggaagaaaggtggcgatttgcacagctcatctttgactgtggagtgttctaagacttcagagagtgaaccaaagaatttcactgctgatcttgggctctccttgacaggggagtcttgcaaactgatgctttcttcaagtgagtatagtcaatcctcaaagatggagagcttggcttctcccaggagtgagggagatggagagaatacccagattgaggacactgaaccgttgtctccagtcaccaattctaaacttcctgctgaaagtgatgatgtcctgatgaatccagcaccagatgaccaagtagaaatgaatcagaatgatgacaaagtaaaagagggtgacacagagaacacaggtgaccatggtgttttagctgctggtagtaaaggcagagaagaaaccgttgctgaagatgtttgcattgatctcacttgtgattctgggagtcaggcagttccgtctcccgctactcggtccgaggcactttctagtgtcttagatcaggaggaagctatggaaattaaagaacagcatccagaggaggggttttcgggatctgaggtagaagaagtccccgagactccctgtgaaagtcacagagaggagcccaaggaagaaacgatggagagtatcccactgcacctttctcttactgaaacacagtcccaggcattgtgtcttcagaaggaaatgcccaaagaagaatgcccagaggccatggaagtggaaccctctgtgattagtgttgactctccccagaagctgccggtactcgaccaggagtcagagcataaggagccagaggcctgggaagaagccgcgtcggaggactcaagtgtggaggactcaagtgtggttatcgttgatgtgaaggagccctcgccaagagttgtcccctgcgaacctttggagggagcagagaaatgctcagattcccactcctgggaggatgtggtgcccgaggtggaaccgtgtgctgcaaatagagtagacactccggaggaaaagattgtagaatgtgacggagatgtgaaagcagagaccacaagaaaggactctgtagaggaagactccccacagcctcctttgccttcagtgaaagacgagcctcccagagacgagcctcccagacccgaccaggagatgcagcagtcccagcttcaagagaaagagagcccagtgaccatagatgcaaaagtggctgatgccaagcagctggagtcagagggagcatcccagcagctttcgaaagcccctgcccgcgactcacaaagtttctgtgaaagttctagtgaaaccccatttcatttcactttgcctaaagaaggtgatattatcccaccattgactggtgcaaccccacctcacattgggcacctaaaattggatcgcaacagacatagtactccaattgggattggcaactatccagaaagcaccatagcaaccagtgatgtcatgtctgaaagcatggtggagatcaatgatcctctacttgggagtgaaaaaggagattctgagtctgccccagaaatggacggaaaactgtatctgaaaatgaaactggttagtcctgagacagaggccagtgaagaatctttgcagtttagcctggaaaagcctgctactgctgagagaaaaaatggatcaactgctgttgctgagcctgttgcaaaaaatggatcaactgctgttgctgagcctgttgccagtccccagaagaccgtgcctgtgtttagctgcaggcaagaggaggtttggagtgaggaccctccctctgtacccatcagggcaaacttgctccattttccaagtgttgaagaagaggacaaagaaaaactggatggtaccccaaagcttaggcagagtgaacagcctgtgaggcccgttgggctggtcaaggatgctgctacttctgaggactctgcttcttctgttccccagcagagagcaacacaggggtcattcagccctcaaggagaagtgatggaaacagacctgctagaaggactgagtgctaaccaggaaaaaccgtgtaaggtcttgatggaaaggcccacccagagtaacataggaatccagaccatggaccattccctgtgtgctccagaaactgtttcagcagcaacccagactgtgaagagtgtatgtgaacaagggaccagtacagtggaccagaactctgggaaacaagatgccactgtgcagaccgagagggggggtgtcgagaaacaggcccctgtggacgatacagaatccctccacagccagggagaagaagaatttgaaatgccccagcctccacatggccatgtcttgcatcgtcacatgagaaccatccgtgaagtccggacacttgtcacccgtgtcatcacagatgtttactatgtggatgggacagaagtggaaaggaaagtaactgaggagactgaagaaccaattgtagaatgtcaggaatgtgaaacagaggtttccccttcccagactggaggctcttctggagacctgggagacatcagttccttctcctccaaagcatccagctcacaccatacatcaagtgggacaagtctctcagccatgcatagcagtggcagctcaggacgaggagccgggccactcaaagggaaaaccagcgggacagaacctgcagattttgctttacccagttcccgaggaggcccaggaaaactgagtcctagaaaagggatcaatcagacaggggcaccagtgtgtgaggaagatggtgatgcaggccttggcatcagacagggagggaaggctcctgtcacacctcgtggtcgtggtcgaaggggccgcccaccttctcggaccactggaacaagagatgcagttgtgtctggtccgttgggcatagaagacatttcacctagcatgtcaccagatgacaagtccttcacccgaattgtgccccgtgtaccagactctaccaaacgaattgataccagttctactgttttgaggcggagtgattccccagagattccttttcaggctgctactgggtcttctgatggcttggatgcctcatctccaggaaatagctttgtcgggctccgtgttgtagccaagtggtcatccaatggctatttttactctgggaaaatcacacgggatgttggagctgggaagtataagctgctcttcgatgatgggtatgaatgtgatgtgctgggcaaagacattctcctgtgtgaccctatacccctggacactgaagtgacagccctctcagaagatgagtatttcagtgcaggagtggtcaaaggacacagaaaggagtctggggagctgtactacagcattgaaaaagaaggccaaaggaagtggtataagcggatggcagtcatcctgtccttggaacaaggaaacaggttaagagagcaatatgggcttggcccatatgaagctgttacacccctcacaaaggcagcagacatcagcctagataatttggtggaaggaaagcggaaacgtcgcagtaacatcagctccccagccacccccactgcctccagcagcagcagcagcagcagcacaacacccacccgtaaaaccacagagagtccccgtgcttccacgggagttccatcaggcaaaaggaaactcatcacttctgaagaggaacggtccccagctaagcgaggccgcaagtctgtcaccgtgaaacctggtacagtgggggcaggagaatttgtgagcccctgcgagagtggagacaacacaggtgaaccttctgtcctggaagagccaagagggcctttgcccctcaacaagaccttgtttctgggctatgcctttctccttaccatggccacaaccaatgacaagctggccagccgctctaaactgctagatggtcctacaggaagcagtgaagaagaggaggaatttttagaaattcctcctttcaacaagcagtatacagaatgccagcttcgagcaggagctgggtatatccttgaagacttcaatgaagcccagtgtaacacagcctaccagtgtctcctaattgctgatcagcattgtcgaacccggaagtacttcctgtgccttgccagtggaattccttgtgtgtctcatgtctgggtccatgacagctgccatgccaaccagctccaaaactaccgtaattatctgctgcctgctgggtatagccttgaggagcagcgaattctggattggcaacctcgtgaaaaccctttccagaatctgaaggtactcttggtgtcagatcaacaacagaacttcctggagctctggtctgagatcctcatgactggaggggcagcctctgtgaagcagcaccattcaagtgcccataacaaagacattgctttaggggtatttgatgtggtggtgacagacccctcatgcccagcctcggtgctcaagtgtgctgaagccttgcagctgcctgtggtgtcacaagagtgggtgatccagtgcctcattgttggggagagaattggattcaagcagcatccaaaatataaacatgattatgtttctcactaa

SEQ ID NO: 30 - DNA, 47 bp, Cricetulus griseus
  misc_feature 1..47: note = ERV group 1 consensus sequence
  source 1..47: mol_type = genomic DNA
gcccccgccatatccgccactgccgcccccaccagaggcagaagcgg

SEQ ID NO: 31 - DNA, 47 bp, Cricetulus griseus; identical to SEQ ID NO: 30
  misc_feature 1..47: note = Group 1 PPYP motif sequence
  source 1..47: mol_type = genomic DNA

SEQ ID NO: 32 - DNA, 37 bp, Cricetulus griseus
  misc_feature 1..36: note = PPYP motif sequence with out-of-frame deletions via PPYP7 sgRNA
  source 1..37: mol_type = genomic DNA
gcccccgccatatccgccccaccagaggcagaagcgg